(19) United States
(12) Patent Application Publication
(10) Pub. No.: US 2025/0326775 A1
(43) Pub. Date: Oct. 23, 2025

(54) A NOVEL THIOL REDUCTANT, PREPARATION METHOD AND USE THEREOF

(71) Applicant: SUZHOU BIOREINNO BIOTECHNOLOGY LIMITED COMPANY, SUZHOU CITY (CN)

(72) Inventors: Lili WU, SUZHOU (CN); Ao JI, SUZHOU (CN); Wenxu HE, SUZHOU (CN); Yicheng WANG, SUZHOU (CN); Yu SHI, SUZHOU (CN); Yajun RAN, SUZHOU (CN)

(21) Appl. No.: 18/995,040

(22) PCT Filed: Aug. 22, 2023
(86) PCT No.: PCT/CN2023/114320

§ 371 Date: Jan. 15, 2025

(30) Foreign Application Priority Data
Aug. 22, 2022 (WO) PCT/CN2022/113992
Sep. 20, 2022 (WO) PCT/CN2022/119955
Sep. 20, 2022 (WO) PCT/CN2022/119999
Nov. 11, 2022 (WO) PCT/CN2022/131521
Jan. 19, 2023 (WO) PCT/CN2023/073070

(51) Int. Cl. C07F 9/50; C07F 9/58

(52) U.S. Cl. CPC C07F 9/5004; C07F 9/58

(57) ABSTRACT

The present disclosure relates to a novel thiol reductant having the formula (I), the preparation and the use in antibody modification.

### CROSS-REFERENCE TO RELATED APPLICATIONS

This application claims the priority to PCT Application No. PCT/CN2022/113992, filed on Aug. 22, 2022, PCT Application No. PCT/CN2022/119999, filed on Sep. 20, 2022, PCT Application No. PCT/CN2022/119955, filed on Sep. 20, 2022, PCT Application No. PCT/CN2022/131521, filed on Nov. 11, 2022, and PCT Application No. PCT/CN2023/073070, filed on Jan. 19, 2023. The contents of the prior PCT applications are considered as a part of the present disclosure and is incorporated herein in its entirety.

### TECHNICAL FIELD

The disclosure relates to a novel thiol reductant, preparation method and use thereof. The thiol reductant could be used in antibody modification.

### BACKGROUND

The statements in this section merely provide background information related to the present disclosure and do not necessarily constitute prior art.

Antibody-drug conjugates (ADCs) are innovative biopharmaceutical products in which a monoclonal antibody is linked to a small molecule drug with a stable linker. ADCs ideally combine the specificity of antibodies and high potency of cytotoxic drugs by delivering potent cytotoxic drugs to antigen-expressing cells, thereby enhancing their targeted cytotoxic activity.

Generally, antibody conjugation to cytotoxic agents commonly involves conjugation to exposed residues including lysines or reduction of disulfide bonds to expose free interchain cysteines on a therapeutic IgG (Immunoglobulin G) antibody. There are other, more recent approaches that introduce conjugation sites to the mAb such as site-specific glycan conjugation, cysteine engineering, incorporation of unnatural amino acids and coupling short peptide tags to drug-linkers. There are typically 80 lysine residues on an antibody; however, less than ten residues are chemically accessible for conjugation. Cysteine conjugation eventuates in the reduction of four interchain disulfide bonds. These bonds are reduced under specific conditions and subsequently result in two, four, six or eight exposed sulfhydryl groups. Both Cys and Lys conjugation methods result in heterogeneous mixtures. (“Advances and Limitations of Antibody Drug Conjugates for Cancer”. Biomedicines. 2021 August; 9(8): 872.).

The drug-antibody ratio (DAR), or number of drug molecules conjugated to a single ADC, is very important for the determination of efficacy of ADCs. DAR widely varies and depends on other ADC variables. The DAR values are also dependent on the site of conjugation and the use of light or heavy conjugated chains. The DAR value influences the effectiveness of the medicine due to the depression in potency caused by low drug loading, while elevated drug loading can impact toxicity and pharmacokinetics (“Introduction to Antibody-Drug Conjugates”. Antibodies (Basel). 2021 December; 10(4): 42.). The conventional non-specific conjugation and conjugate distribution are largely influenced by factors such as pH, concentration, salt concentration, and co-solvents, so establishing a robust conjugation process always is challenging.

A number of methods have been developed to improve the homogeneity of ADCs. For example, Genentech's THIOMAB technology is developed based on improve the homogeneity of ADCs through antibody engineering, by introducing cysteine in the primary sequence of the antibody and realizing site-directed coupling to improve the uniformity of the product (“Cysteine-Based Coupling: Challenges and Solutions”. Bioconjug Chem. 2021 Aug. 18; 32(8):1525-1534.).

US20210040145 discloses a 14-amino acid peptide Tub-tagf used to the C-terminus of any POI and catalyzes the addition of a variety of different tyrosine derivatives. Taking advantage of this enzyme, Tub-tag technology repurposed tubulin-tyrosine ligase for the attachment of functional moieties at the C-terminus of antibody to homogeneously generate antibody conjugates with DAR 2.

However, those technologies involve protein engineering and/or enzyme catalysis, so that those technologies suffer from several drawbacks, such as lower level of antibody expression, immunogenicity risk, complicated purification, and/or high cost.

Therefore, antibody-drug conjugates with improved homogeneity, could provide benefits in terms of better stability and lower immunogenicity, and further result in therapeutic benefits, for example, better efficacy and lower toxicity. So, novel reductant and processes for preparing ADCs with high homogeneity are highly desirable and long-term pursuit.

### SUMMARY

For the above-mentioned purpose, provided herein is a reductant having the following formula (I):

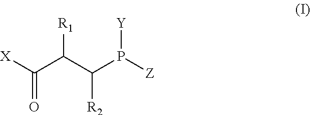

or a salt, solvate, stereoisomer thereof, which characterized in that,
R₁ is H, —NH₂, —C(O)(R₃R₄), optionally substituted C₁-C₅ alkyl group, optionally substituted C₁-C₅ hydroxyalkyl group, or optionally substituted aryl group;
R₃ is N, NH or O;
R₄ is H, optionally substituted C₁-C₅ alkyl group, optionally substituted C₁-C₅ hydroxyalkyl group, or optionally substituted aryl group;
R₂ is H, optionally substituted C₁-C₅ alkyl group, or optionally substituted C₁-C₅ hydroxyalkyl group;
X is OH, optionally substituted C₁-C₅ alkoxy group or —NR₅R₆,
R₅ and R₆ independently are H, C₀-C₅ hydroxyalkyl group, optionally substituted C₁-C₅ alkyl group, optionally substituted C₂-C₅ carboxy alkyl group, optionally substituted C₁-C₅ alkoxy group, optionally substituted heteroaryl alkyl group, optionally substituted aryl alkoxy group, optionally substituted arylalkyl group, optionally substituted aryl group, C₁-C₅ alkyl sulfonyl group, or —(CH₂)n¹(OCH₂CH₂O)n²CH(R₈)CO(R₇),
R₇ is C₀-C₅ hydroxyalkyl group, —NHOH,
R₈ is H, optionally substituted arylalkyl group,
n¹ and n² independently are the number 0, 1, 2, 3, 4,
Y is the same as X, or Y is an ester or amide of X,
Z is the same as X or Y, or
Y and Z independently are selected from the group consisting of

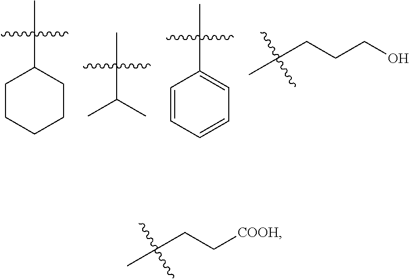

X, Y and Z are not

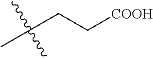 [SMILES: *CCC(=O)O]

at the same time.

In one aspect, provided herein is a composition comprising a reductant described above and transition metal ions. The transition metal ions are Zn²⁺, Cd²⁺, Hg²⁺, Ni²⁺, Co²⁻ or the combination thereof.

In one aspect, provided herein is a method of preparing the reductant described above, which characterized in that, at least one X′ is connected to a compound of formula II by introducing a condensation reagent under an inert atmosphere,

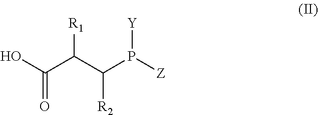

R₁ is H, —NH₂, —C(O)(R₃R₄), optionally substituted C₁-C₅ alkyl group, optionally substituted C₁-C₅ hydroxyalkyl group, or optionally substituted aryl group;
R₃ is N, NH or O;
R₄ is H, optionally substituted C₁-C₅ alkyl group, optionally substituted C₁-C₅ hydroxyalkyl group, or optionally substituted aryl group;
R₂ is H, optionally substituted C₁-C₅ alkyl group, or optionally substituted C₁-C₅ hydroxyalkyl group;
X′ is optionally substituted C₁-C₅ alkyl alcohol or NR₅R₆,
R₅ and R₆ independently are H, C₀-C₅ hydroxyalkyl group, optionally substituted C₁-C₅ alkyl group, optionally substituted C₂-C₈ carboxy alkyl group, optionally substituted C₁-C₅ alkoxy group, optionally substituted heteroaryl alkyl group, optionally substituted aryl alkoxy group, optionally substituted arylalkyl group, optionally substituted aryl group, C₁-C₅ alkyl sulfonyl group, or —(CH₂)n¹(OCH₂CH₂O)n²CH(R₈)CO(R₇),
R₇ is C₀-C₅ hydroxyalkyl group, —NHOH,
R₈ is H, optionally substituted arylalkyl group,
n¹ and n² independently are the number 0, 1, 2, 3, 4,
Y is the same as X, or Y is an ester or amide of X,
Z is the same as X or Y, or
Y and Z independently are selected from the group consisting of

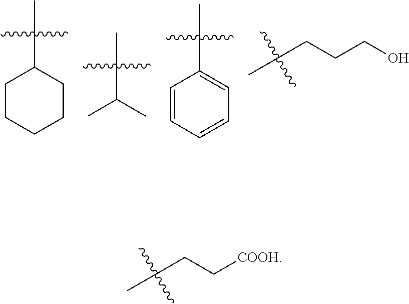

In one aspect, provided herein is use of the reductant described above or the composition described above in reducing the interchain disulfide bonds of an antibody.

In one aspect, provided herein is a method of preparing an antibody with site-specific modification, comprising steps of

(A1) incubating a reductant described above as a first reductant and the transition metal ions in the presence of an antibody in a buffer system to selectively the reduce interchain disulfide bonds within the antibody to afford the antibody bearing reduced thiol groups.

In some embodiments, two interchain disulfide bonds in Fab region of the antibody and one interchain disulfide bonds in hinge region of the antibody are reduced.

In some embodiments, the method further comprising step of

(A2) introducing oxidant to selectively re-oxidize the reduced thiol groups resulted from step (A1), optionally re-oxidize the reduced thiol groups in Fab region of the antibody.

In some embodiments, the method further comprising step of

(A3) incubating a second reductant in a buffer system to selectively reduce the interchain disulfide bonds resulted from step (A2), optionally reduce the interchain disulfide bonds in the hinge region of the antibody.

In some embodiments, the method further comprising the following steps,

(B1) introducing metal chelators and first payload units to react with the reduced thiol groups resulted from step (A1), step (A2) or step (A3), wherein, the first payload unit is an end capping reagent, a first linker-payload or a first thio-bridging reagent, optionally, the first thio-bridging reagent bears the first linker-payload or reactive groups.

In some embodiments, the method further comprising step of

(B2) incubating a second reductant in a buffer system to reduce the interchain disulfide bonds resulted from step (B1), optionally, introducing the transition metal ions; and

(B3) introducing second payload units to react with the reduced thiol groups resulted from step (B2), optionally, introducing the metal chelators, wherein, the second payload unit is a second linker-payload or a second thio-bridging reagent, optionally, the second thio-bridging reagent bears the second linker-payload of reactive groups.

In one aspect, provided herein is a modified antibody prepared by the method described above.

In some embodiments, the modified antibody is the antibody with site-specific modification, optionally, the modified antibody comprises the ADC with D2, the ADC with D4, the ADC with D1, the ADC with D6, the ADC with D3, the ADC with D1+D6, the ADC with D1+D2, the ADC with D1+D4, the ADC with D2+D4, the ADC with D6+D2, the ADC with D6+D1, the ADC with D3+D1, the ADC with D3+D2, the ADC with D0+D6, or the ADC with D0+D2.

In one aspect, provided herein is a pharmaceutical composition comprising an antibody with site-specific modification prepared by the method described above, and at least one pharmaceutically acceptable ingredient.

In one aspect, provided herein is use of the antibody with site-specific modification prepared by the method described above or the pharmaceutical composition described above in the manufacture of a therapeutic agent for preventing, diagnosing or treating a disease.

In one aspect, provided herein is a method of preventing or treating a disease in a subject in need thereof, comprising administrating to the subject a therapeutically effective amount of an antibody with site-specific modification prepared by the method described above.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows HIC-HPLC chromatogram of Trastuzumab-[MC-VC-PAB-MMAE]₆ conjugate prepared of Example 32. HIC-HPLC is short for Hydrophobic interaction chromatography-High performance liquid chromatography.

FIG. 2 shows HIC-HPLC chromatogram of Trastuzumab-[MC-VC-PAB-MMAE]₆ conjugate prepared of Example 33.

FIG. 3 A-H show HIC-HPLC chromatogram of Trastuzumab-[MC-VC-PAB-MMAE]₆ conjugate prepared of Examples 34-41, wherein, the reductant is TCEP-1, TCEP-2, TCEP-3, TCEP-4, TCEP-5, TCEP-6, TCEP-7 and TCEP-8.

FIG. 4 A-H show HIC-HPLC chromatogram of Trastuzumab-[MC-VC-PAB-MMAE]₆ conjugate prepared of Examples 42-49, wherein, the reductant is TCEP-9, TCEP-10, TCEP-15, TCEP-18, TCEP-19, TCEP-20, TCEP-23 and TCEP-24.

FIG. 5 A-H show HIC-HPLC chromatogram of Trastuzumab-[MC-VC-PAB-MMAE]₆ conjugate prepared of Examples 50-57, wherein, the reductant is TCEP-25, TCEP-26, TCEP-28, TCEP-A, TCEP-A, TCEP-30, TCEP-31 and TCEP-33.

FIG. 6 shows HIC-HPLC chromatogram of Trastuzumab-[MC-VC-PAB-MMAE]₆ conjugate prepared of Comparative example 5, wherein, the reductant is TCEP.

FIG. 7 A-B show HIC-HPLC chromatogram of Trastuzumab-[MC-VC-PAB-MMAE]₆ conjugate prepared of examples 58-59, wherein, the molar ratio of the reductant and the antibody is 2.8:1, 3.5:1.

FIG. 8 shows HIC-HPLC chromatogram of Trastuzumab-[MC-VC-PAB-MMAE]₆ conjugate prepared of Example 60.

FIG. 9 shows HIC-HPLC chromatogram of Trastuzumab-[MC-VC-PAB-MMAE]₆ conjugate prepared of Example 61.

FIG. 10 shows HIC-HPLC chromatogram of Trastuzumab-[MC-VC-PAB-MMAE]₆ conjugate prepared of Example 62.

FIG. 11 shows HIC-HPLC chromatogram of Trastuzumab-[MC-VC-PAB-MMAE]₆ conjugate prepared of Example 63.

FIG. 12 shows HIC-HPLC chromatogram of Trastuzumab-[MC-VC-PAB-MMAE]₆ conjugate prepared of Example 64.

FIG. 13 shows HIC-HPLC chromatogram of Trastuzumab-[MC-VC-PAB-MMAE]₆ conjugate prepared of Example 65.

FIG. 14 shows HIC-HPLC chromatogram of Trastuzumab-[MC-VC-PAB-MMAE]₆ conjugate prepared of Example 66.

FIG. 15 shows HIC-HPLC chromatogram of Trastuzumab-[MC-VC-PAB-MMAE]₆ conjugate prepared of Example 67.

FIG. 16 shows HIC-HPLC chromatogram of Trastuzumab-[MC-VC-PAB-MMAE]₆ conjugate prepared of Example 68.

FIG. 17 shows HIC-HPLC chromatogram of Trastuzumab-[MC-VC-PAB-MMAE]₆ conjugate prepared of Example 69.

FIG. 18 A-C show HIC-HPLC chromatogram of Trastuzumab-[MC-VC-PAB-MMAE]₆ conjugate prepared of examples 70-72, wherein, the molar ratio of the reductant and the antibody is 5:1, 6:1, 7:1.

FIG. 19 A-F show HIC-HPLC chromatogram of Trastuzumab-[MC-VC-PAB-MMAE]₆ conjugate prepared of examples 73-78, wherein, the molar ratio of the reductant and the antibody is 8:1, 9:1, 10:1, 11:1, 12:1, 13:1.

FIG. 20 shows HIC-HPLC chromatogram of Trastuzumab-[MC-VC-PAB-MMAE]₆ conjugate prepared of Comparative example 1, wherein, the molar ratio of the transition metal ions and the reductant is 0:1.

FIG. 21 A-C show HIC-HPLC chromatogram of Trastuzumab-[MC-VC-PAB-MMAE]₆ conjugate prepared of Comparative examples 2-4, wherein, the molar ratio of the transition metal ions and the reductant is 0:1.

FIG. 22 shows HIC-HPLC chromatogram of Trastuzumab-[MC-VC-PAB-MMAE]₆ conjugate prepared of Example 79.

FIG. 23 shows HIC-HPLC chromatogram of Trastuzumab-[MC-VC-PAB-MMAE]₆ conjugate prepared of Example 80.

FIG. 24 shows HIC-HPLC chromatogram of Trastuzumab-[MC-VC-PAB-MMAE]₆ conjugate prepared of Example 81.

FIG. 25 shows HIC-HPLC chromatogram of Trastuzumab-[MC-VC-PAB-MMAE]₆ conjugate prepared of Example 82.

FIG. 26 shows HIC-HPLC chromatogram of Trastuzumab-[MC-VC-PAB-MMAE]₆ conjugate prepared of Example 83.

FIG. 27 shows HIC-HPLC chromatogram of Trastuzumab-[MC-VC-PAB-MMAE]₆ conjugate prepared of Example 84.

FIG. 28 shows HIC-HPLC chromatogram of Trastuzumab-[MC-VC-PAB-MMAE]₆ conjugate prepared of Example 85.

FIG. 29 A-E show HIC-HPLC chromatogram of Trastuzumab-[MC-VC-PAB-MMAE]₆ conjugate prepared of examples 86-90, wherein, the molar ratio of the transition metal ions and the reductant is 7.5:1, 15:1, 30:1, 0.22:1, 3.33:1.

FIG. 30 shows HIC-HPLC chromatogram of Trastuzumab-[MC-VC-PAB-MMAE]₆ conjugate prepared of Example 91.

FIG. 31 shows HIC-HPLC chromatogram of Trastuzumab-[MC-VC-PAB-MMAE]₆ conjugate prepared of Example 92.

FIG. 32 shows HIC-HPLC chromatogram of Trastuzumab-[MC-VC-PAB-MMAE]₆ conjugate prepared of Example 93.

FIG. 33 shows HIC-HPLC chromatogram of Trastuzumab-[MC-VC-PAB-MMAE]₆ conjugate prepared of Example 94.

FIG. 34 shows HIC-HPLC chromatogram of Trastuzumab-[MC-VC-PAB-MMAE]₆ conjugate prepared of Example 95.

FIG. 35 A-G show HIC-HPLC chromatogram of Trastuzumab-[MC-VC-PAB-MMAE]₆ conjugate prepared of Examples 96-102, wherein, the buffer system is different.

FIG. 36 A-E show HIC-HPLC chromatogram of Trastuzumab-[MC-VC-PAB-MMAE]₆ conjugate prepared of Examples 103-107, wherein, the buffer system is different.

FIG. 37 A-C show HIC-HPLC chromatogram of Trastuzumab-[MC-VC-PAB-MMAE]₆ conjugate prepared of Examples 108-110, wherein, the concentration of the buffer system is different.

FIG. 38 A-F show HIC-HPLC chromatogram of Trastuzumab-[MC-VC-PAB-MMAE]₆ conjugate prepared of Examples 111-116, wherein, the incubation time in step (1) is different.

FIG. 39 A-C show HIC-HPLC chromatogram of Trastuzumab-[MC-VC-PAB-MMAE]₆ conjugate prepared of Examples 117-119, wherein, the incubation time and temperature in step (1) is different.

FIG. 40 shows HIC-HPLC of Trastuzumab-[Bismaleimide-DBCO]₃ conjugate of example 120.

FIG. 41 A shows HIC-HPLC of Trastuzumab-[MC-VC-PAB-MMAE]₆ conjugate of example 121; B shows HIC-HPLC of Trastuzumab-[MC-VC-PAB-MMAE]₆ [MC-GGFG-DXd]₂ conjugate of example 121.

FIG. 42 A shows HIC-HPLC of Trastuzumab-[MC-GGFG-DXd]₆ conjugate of example 122; B shows HIC-HPLC of Trastuzumab-[MC-GGFG-DXd]₆[Maleimide-PEG4-N3-DBCO-Cy3]₁ conjugate of example 122.

FIG. 43 A shows HIC-HPLC of Trastuzumab-[Maleimide]₆ conjugate of example 123; B shows HIC-HPLC of Trastuzumab-[Maleimide]₆[MC-VC-PAB-MMAE]₂ conjugate of example 123.

FIG. 44 shows HIC-HPLC of Trastuzumab-[Maleimide]₆[Maleimide-PEG4-N3-DBCO-Cy3]₁ conjugate of example 124.

FIG. 45 shows HIC-HPLC chromatogram of Trastuzumab-[MC-VC-PAB-MMAE]₂ conjugate prepared of Example 125.

FIG. 46 shows HIC-HPLC chromatogram of Trastuzumab-[MC-VC-PAB-MMAE]₂ conjugate prepared of Example 126.

FIG. 47 shows HIC-HPLC chromatogram of Trastuzumab-[MC-VC-PAB-MMAE]₂ conjugate prepared of Example 127.

FIG. 48 shows HIC-HPLC chromatogram of Trastuzumab-[MC-VC-PAB-MMAE]₂ conjugate prepared of Example 128.

FIG. 49 shows HIC-HPLC chromatogram of Trastuzumab-[MC-VC-PAB-MMAE]₂ conjugate prepared of Example 129.

FIG. 50 shows HIC-HPLC chromatogram of Trastuzumab-[MC-VC-PAB-MMAE]₂ conjugate prepared of Example 130.

FIG. 51 shows HIC-HPLC chromatogram of Trastuzumab-[MC-VC-PAB-MMAE]₂ conjugate prepared of Example 131.

FIG. 52 shows HIC-HPLC chromatogram of Trastuzumab-[MC-VC-PAB-MMAE]₂ conjugate prepared of Example 132.

FIG. 53 shows HIC-HPLC chromatogram of Trastuzumab-[MC-VC-PAB-MMAE]₂ conjugate prepared of Example 133.

FIG. 54 A-H show HIC-HPLC chromatogram of Trastuzumab-[MC-VC-PAB-MMAE]₂ conjugate prepared of Examples 134-141, wherein, the molar ratio of DHAA and the antibody, the molar ratio of the reductant and the antibody, and/or the incubation time in step (1) is different.

FIG. 55 A-F show HIC-HPLC chromatogram of Trastuzumab-[MC-VC-PAB-MMAE]₂ conjugate prepared of Examples 142-147, wherein, the molar ratio of DHAA and the antibody and/or the molar ratio of the reductant and the antibody is different.

FIG. 56 A-C show HIC-HPLC chromatogram of Trastuzumab-[MC-VC-PAB-MMAE]₂ conjugate prepared of Examples 148-150, wherein, the oxidation temperature and time are different.

FIG. 57 A-H show HIC-HPLC chromatogram of Trastuzumab-[MC-VC-PAB-MMAE]₂ conjugate prepared of Examples 151-158, wherein, the buffer system is different.

FIG. 58 A-H show HIC-HPLC chromatogram of Trastuzumab-[MC-VC-PAB-MMAE]₂ conjugate prepared of Examples 159-166, wherein, the buffer system is different.

FIG. 59 shows HIC-HPLC chromatogram of Trastuzumab-[MC-VC-PAB-MMAE]₂ conjugate prepared of Comparative example 6.

FIG. 60 shows HIC-HPLC of Trastuzumab-[Malcimidc-PEG4-N3-DBCO-Cy3]₁ conjugate of example 167.

FIG. 61 shows HIC-HPLC of Trastuzumab-[Maleimide-PEG4-N3-DBCO-Cy3]₁[MC-VC-PAB-MMAE]₆ conjugate of example 168.

FIG. 62 shows HIC-HPLC of Trastuzumab-[Maleimide-PEG4-N3-DBCO-Cy3]₁[MC-VC-PAB-MMAE]₂ conjugate of example 169.

FIG. 63 A shows HIC-HPLC of Trastuzumab-[Maleimide-PEG4-N3-DBCO-Cy3]₁ conjugate of example 170; B-C show HIC-HPLC of Trastuzumab-[Maleimide-PEG4-N3-DBCO-Cy3]₁[MC-VC-PAB-MMAE]₄ conjugate of examples 170-171.

FIG. 64 A shows HIC-HPLC of Trastuzumab-[MC-GGFG-DXd]₂ conjugate of example 172; B shows HIC-HPLC of Trastuzumab-[MC-GGFG-DXd]₂[MC-VC-PAB-MMAE]₄ conjugate of example 172.

FIG. 65 A shows HIC-HPLC of Trastuzumab-[MC-VC-PAB-MMAE]2 conjugate of example 173; B-C show HIC-HPLC of Trastuzumab-[MC-VC-PAB-MMAE]₂[MC-GGFG-DXd]₄ conjugate of examples 173-174.

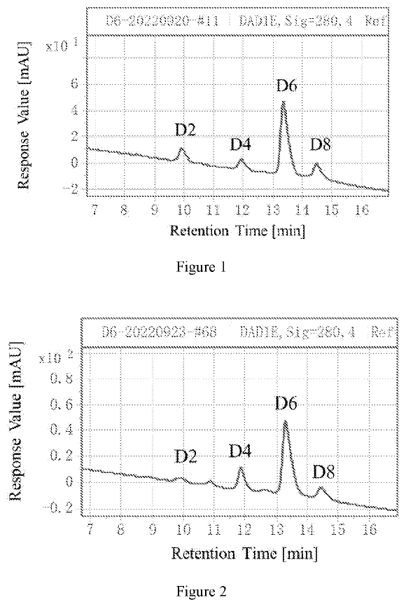

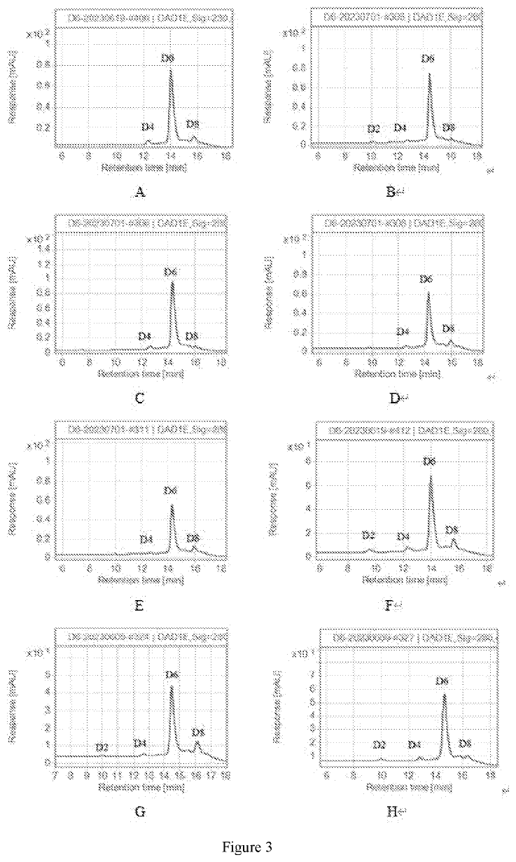

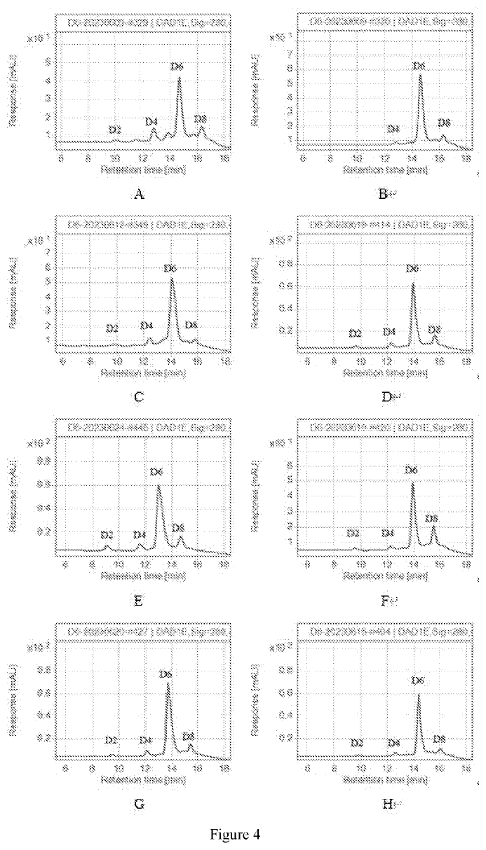

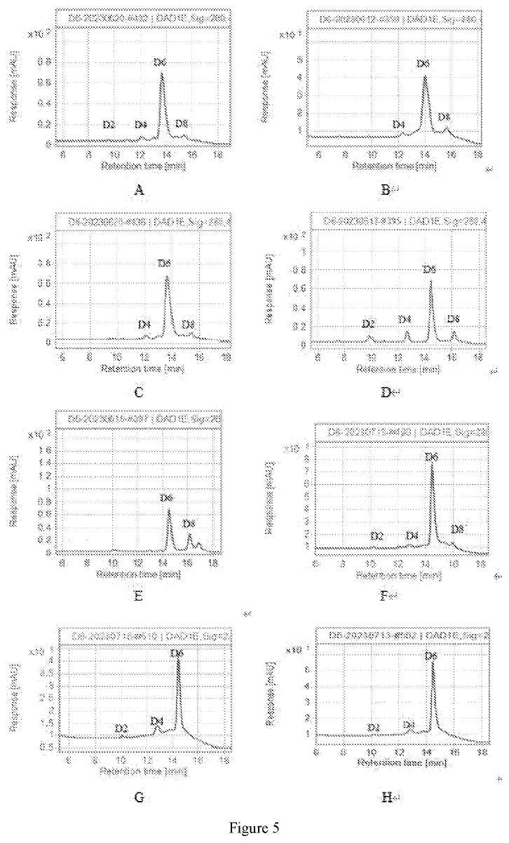

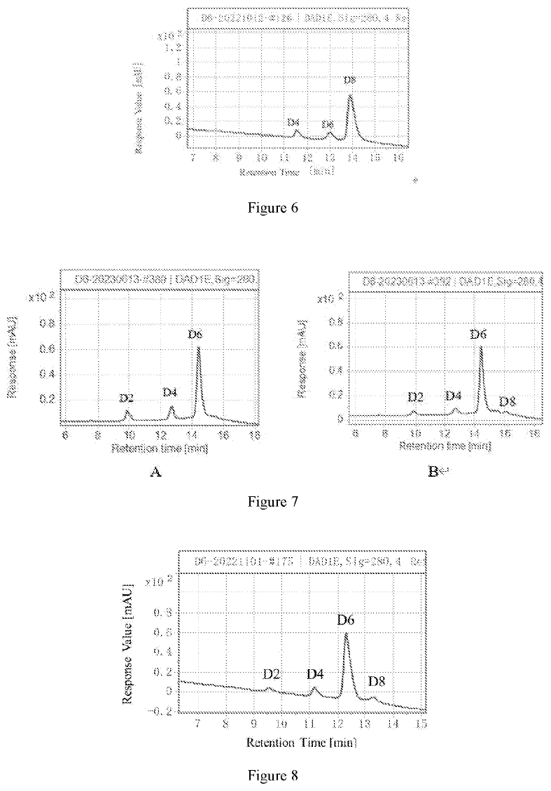

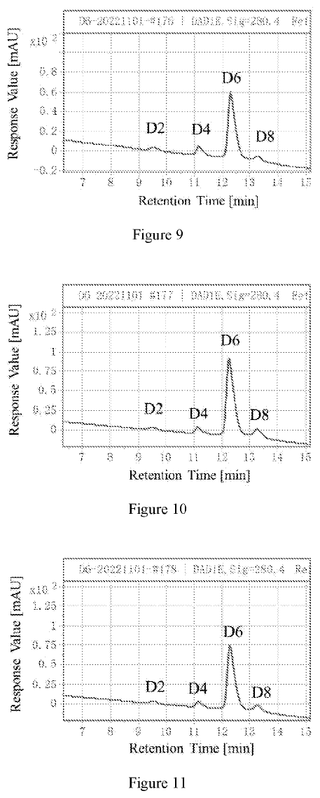

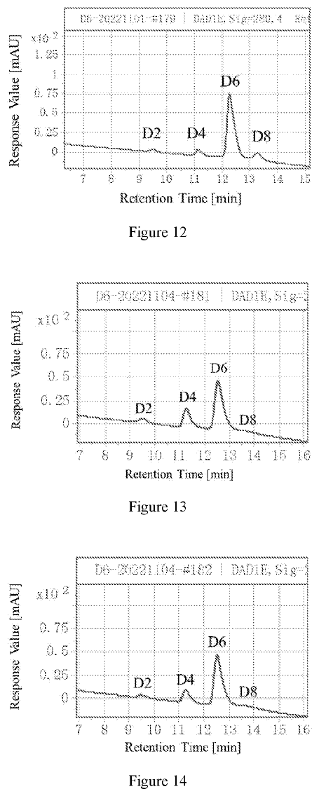

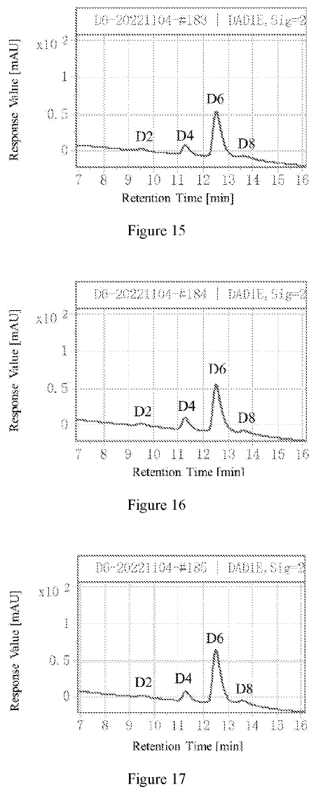

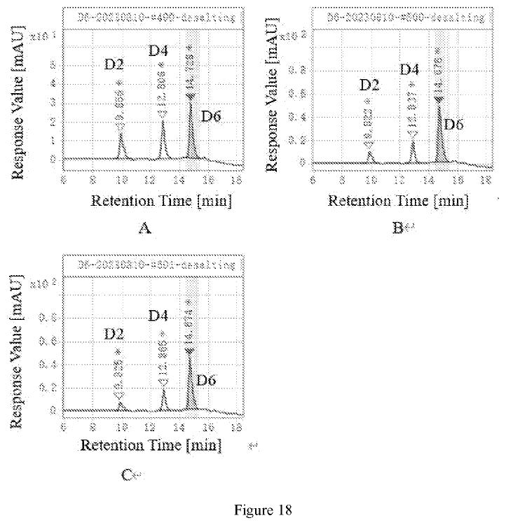

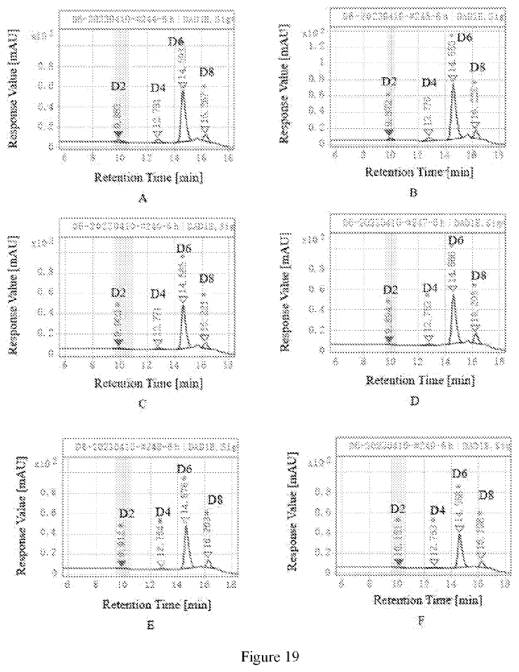

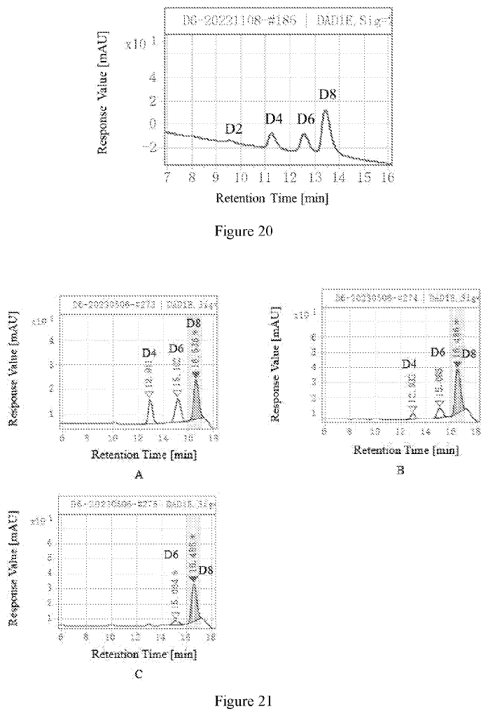

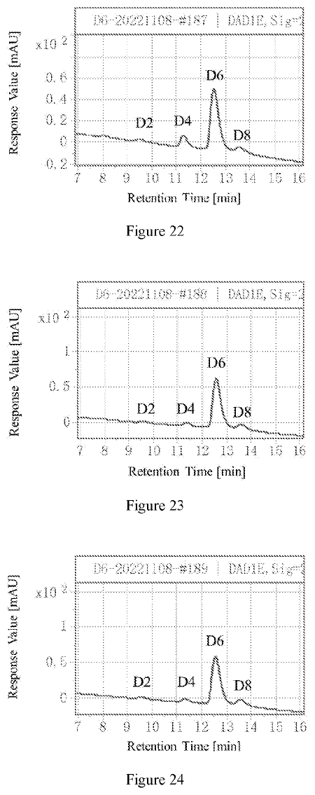

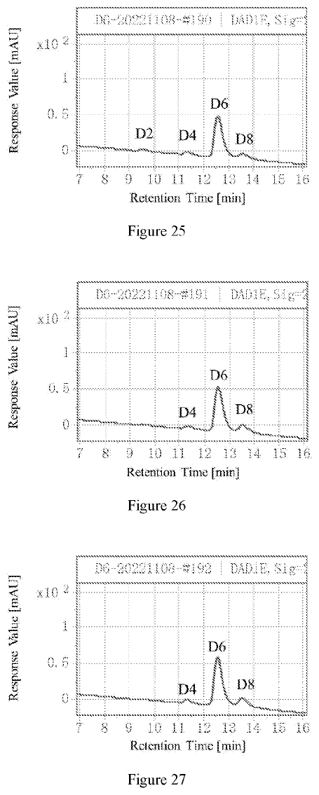

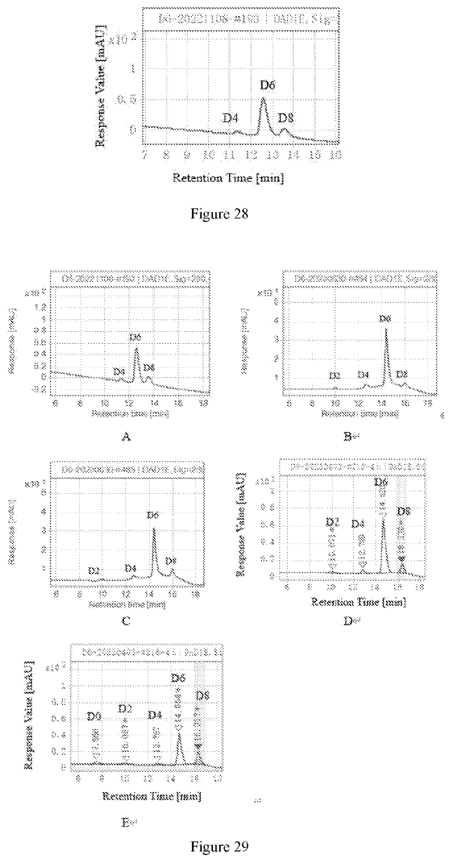

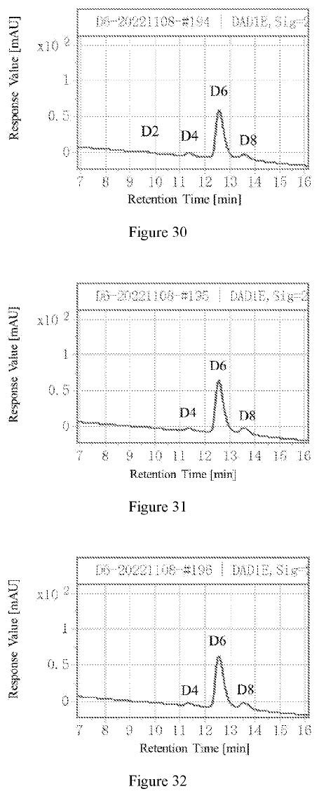

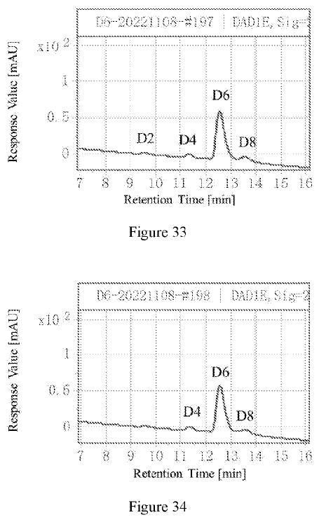

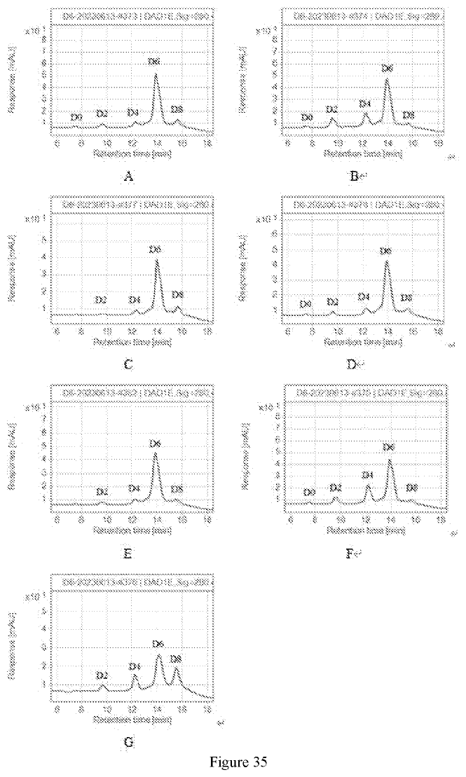

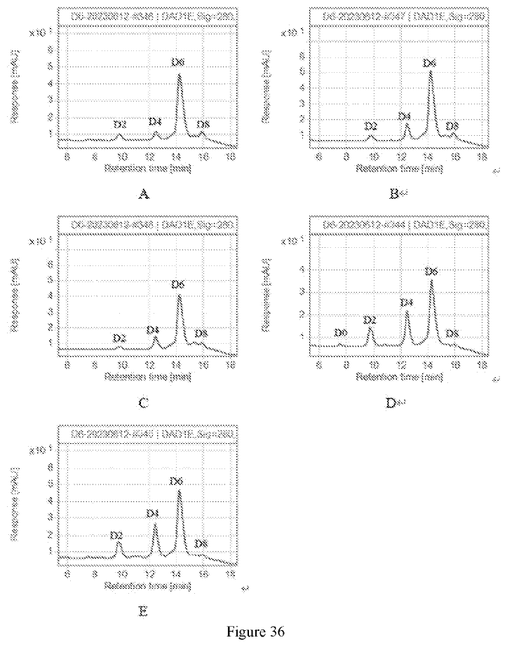

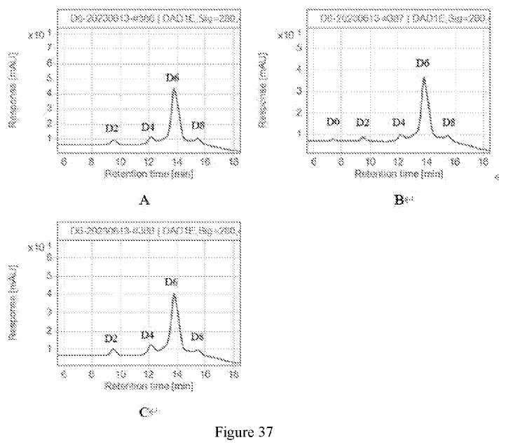

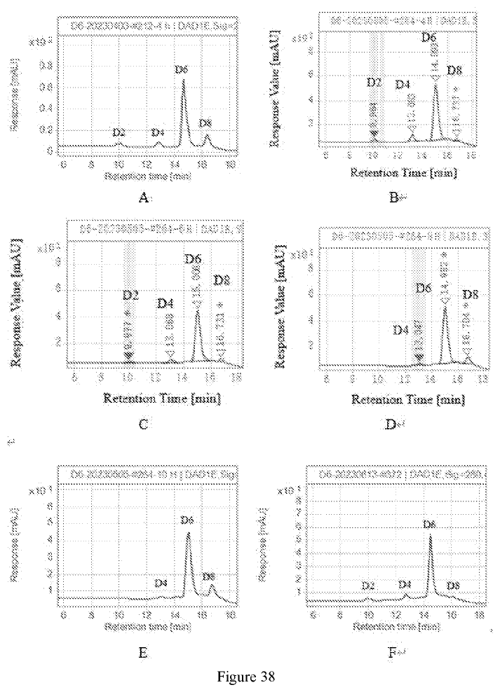

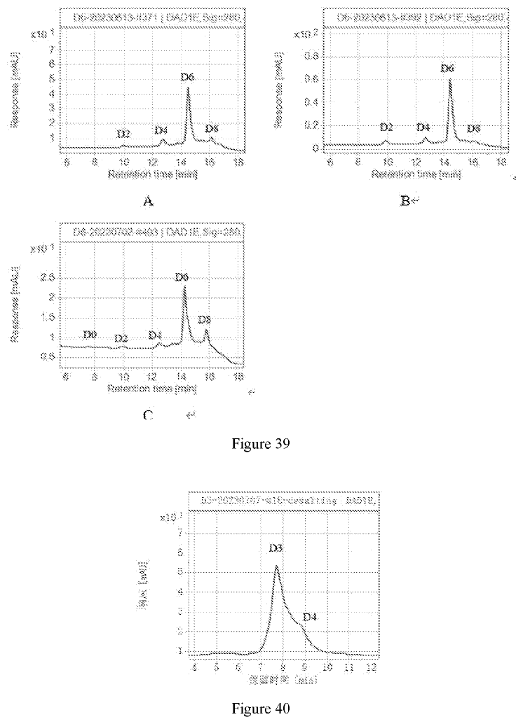

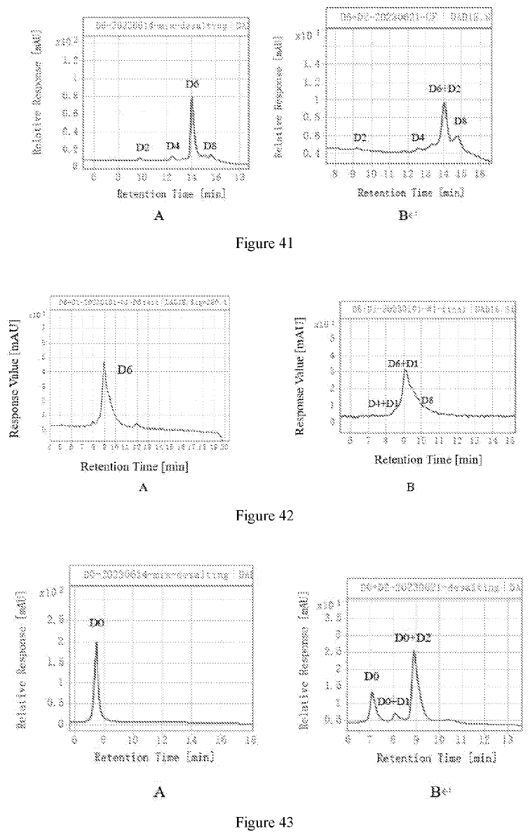

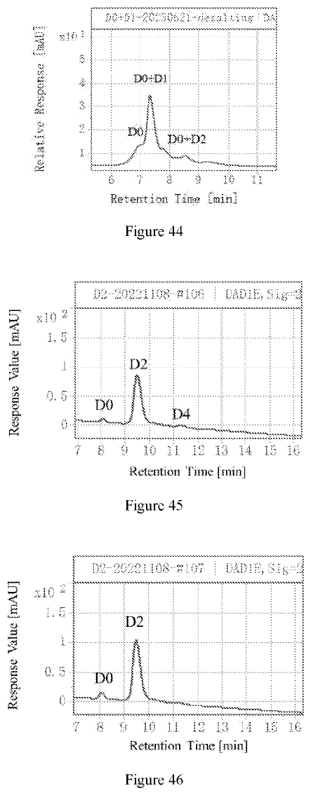

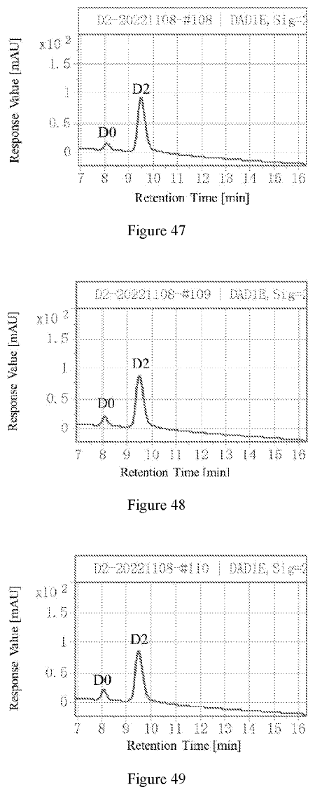

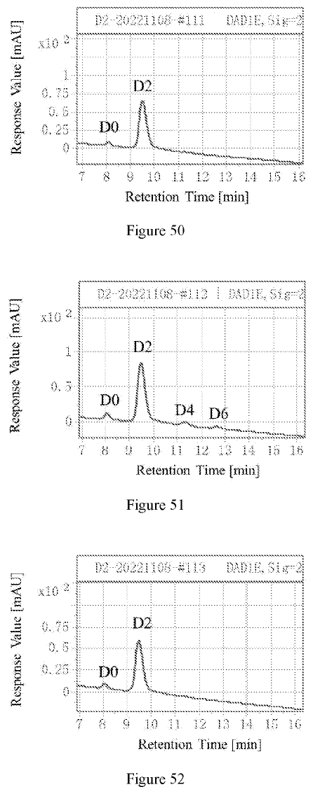

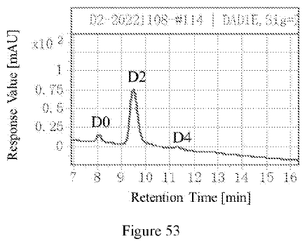

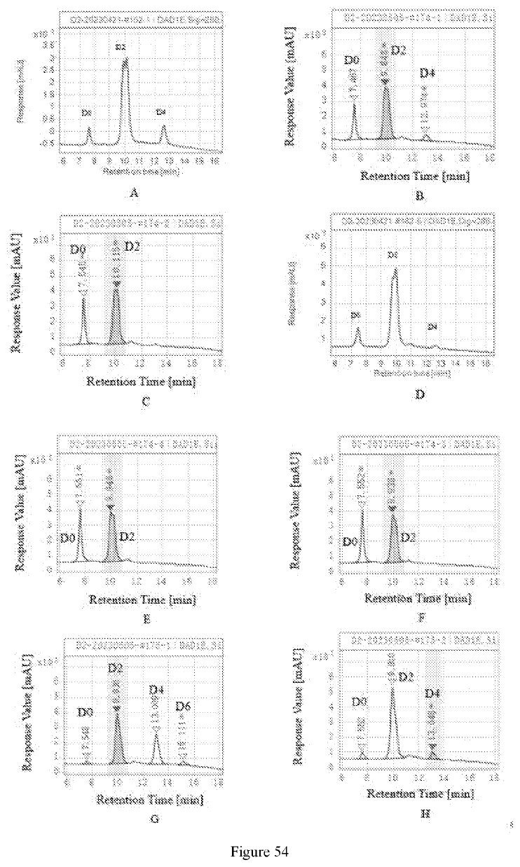

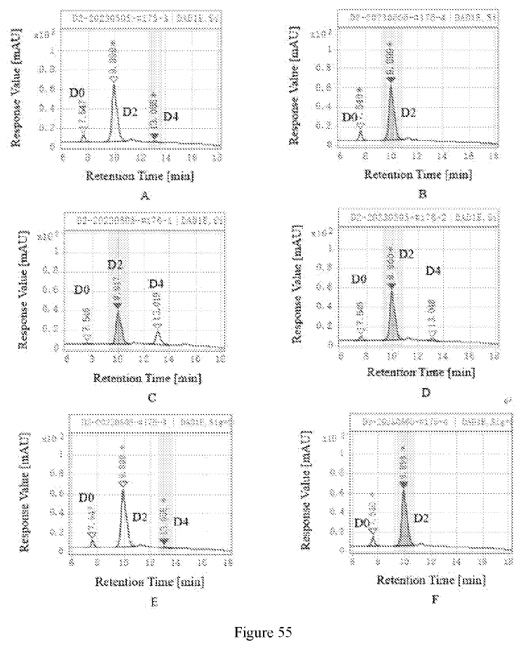

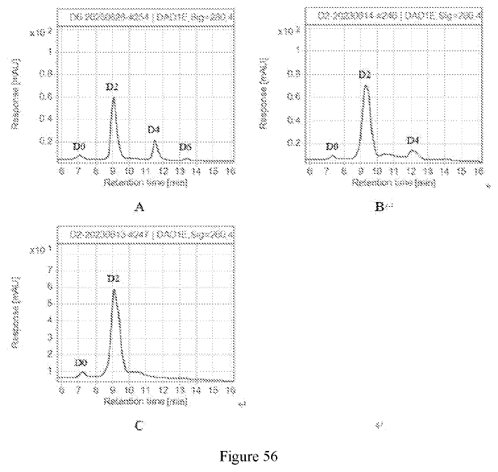

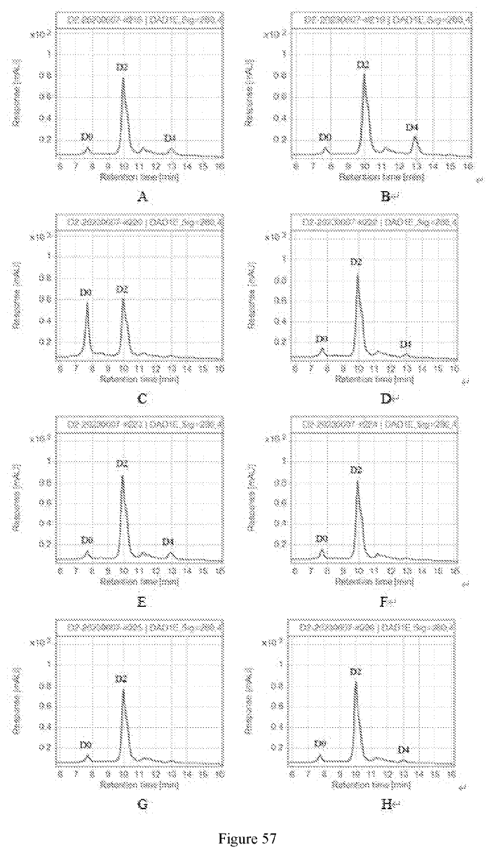

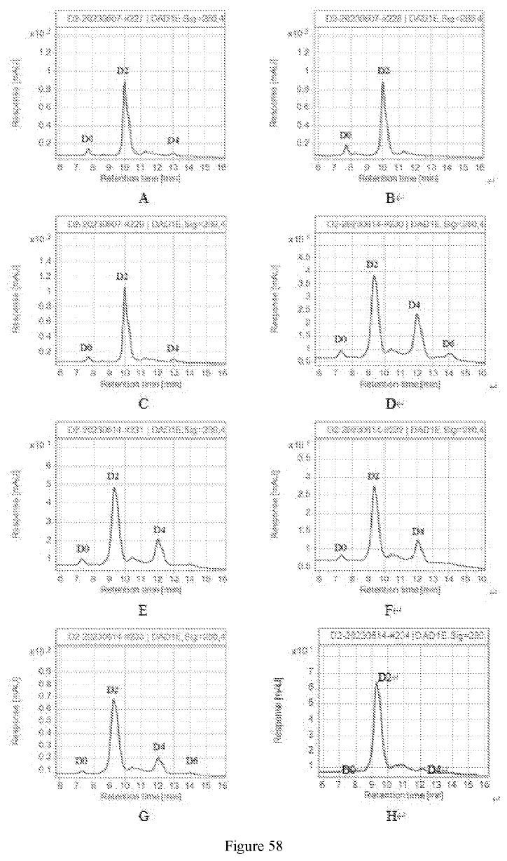

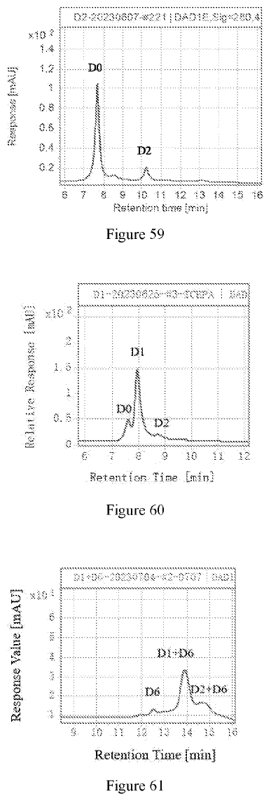

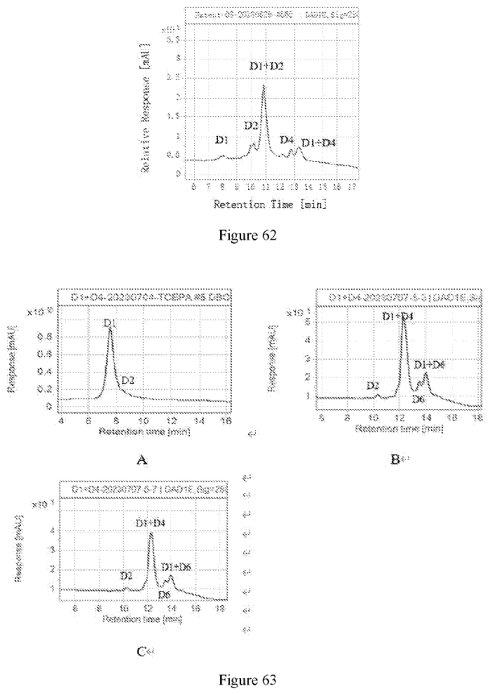

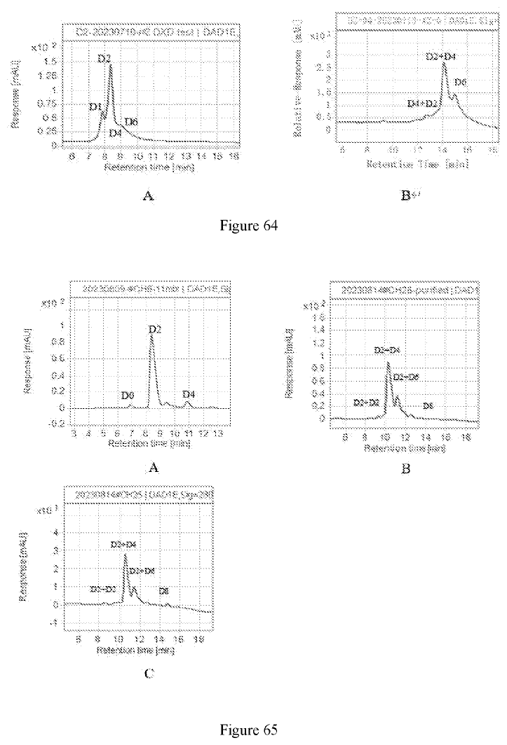

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

### Reductant

The present disclosure provides examples of reductant when preparing antibody-drug conjugates (ADCs).

Provided herein is a reductant having the following formula (I):

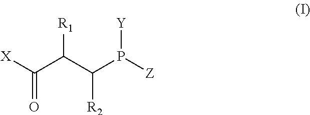

or a salt, solvate, stereoisomer thereof, which characterized in that,
R₁ is H, —NH₂, —C(O)(R₃R₄), optionally substituted C₁-C₅ alkyl group, optionally substituted C₁-C₅ hydroxyalkyl group, or optionally substituted aryl group;
R₃ is N, NH or O;
R₄ is H, optionally substituted C₁-C₅ alkyl group, optionally substituted C₁-C₅ hydroxyalkyl group, or optionally substituted aryl group;
R₂ is H, optionally substituted C₁-C₅ alkyl group, or optionally substituted C₁-C₅ hydroxyalkyl group;
X is OH, optionally substituted C₁-C₅ alkoxy group or —NR₅R₆,
R₅ and R₆ independently are H, C₀-C₅ hydroxyalkyl group, optionally substituted C₁-C₅ alkyl group, optionally substituted C₂-C₅ carboxy alkyl group, or optionally substituted C₁-C₅ alkoxy group, optionally substituted heteroaryl alkyl group, optionally substituted aryl alkoxy group, optionally substituted arylalkyl group, optionally substituted aryl group, C₁-C₅ alkyl sulfonyl group, —(CH₂)n¹(OCH₂CH₂O)ₙ²CH(R₈)CO(R₇),
R₇ is C₀-C₅ hydroxyalkyl group, —NHOH,
R₈ is H, optionally substituted arylalkyl group,
n¹ and n² independently are the number 0, 1, 2, 3, 4,
Y is the same as X, or Y is an ester or amide of X,
Z is the same as X or Y, or
Y and Z independently are selected from the group consisting of

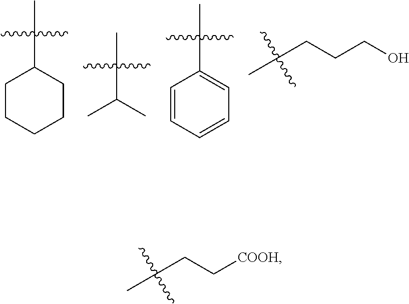

X, Y and Z are not

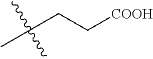 [SMILES: *CCC(=O)O]

at the same time.

The term “C₁-C₅ alkyl group” refers to an aliphatic hydrocarbon group which having 1 to 3 carbon atoms in the chain or cyclic. Exemplary alkyl groups include methyl, ethyl, n-propyl and i-propyl.

The term “C₀-C₅ hydroxyalkyl group” refers to hydroxy group or C₁-C₅ alkyl group, wherein one or several H atoms are substituted with one, two or three hydroxy groups. Exemplary C₁-C₅ hydroxyalkyl group is hydroxy methyl group, 2-hydroxy ethyl group, 3-hydroxy propyl group.

The term “C₂-C₈ carboxy alkyl group” refers to a C₂-C₈ alkyl group which is substituted with one two, three, four, five, six or seven carboxy groups. Exemplary C₂-C₈ carboxy alkyl group is —COOH, —CH₂COOH, —CH₂CH₂COOH, —CH₂(CH₂)₂COOH, —CH₂(CH₂)₃COOH, —CH₂(CH₂)₄₄COOH, —CH₂(CH₂)₅COOH, —CH₂(CH₂)₆COOH or —CH₂(CH₃)COOH.

The term “C₁-C₅ alkyl sulfonyl group” refers to a C₁-C₅ alkyl group, wherein one or several H atoms are substituted with one, two or three sulfonyl group. Exemplary C₁-C₅ alkyl sulfonyl group is —CH₂S(O)₂OH, —CH₂CH₂S(O)₂OH or —CH₂(CH₂)₂S(O)₂OH.

The term “aryl group” refers to an aromatic or hetero aromatic group, composed of one or several rings, comprising three to fourteen carbon atoms, preferentially six to ten carbon atoms. Exemplary aryl group is phenyl group.

The term “aryl group” also refers to an aromatic group, wherein one or several H atoms are replaced independently by other group, such as F, Cl, Br, I, hydroxy, carboxy, sulfonyl, amino, methoxy or ethoxy, N-hydroxy formamide group, N-hydroxy acetamido group, 4-pyridyl group, 2-pyridyl group,

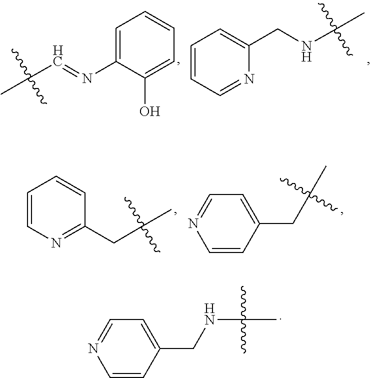

The term “heteroaryl group” refers to one or several carbon on aromatic group, preferentially one, two, three or four carbon atoms are replaced by O, N, Si, Se, P or S, preferentially by O, S, N. Exemplary heteroaryl group is imidazolyl group, pyridyl group, bipyridyl group, quinolinyl group, iso-quinolinyl group.

The term “heteroaryl group” also refers to hetero aromatic group, wherein one or several H atoms are replaced independently by other group, such as F, Cl, Br, I, hydroxy, carboxy, amino, hydroxyalkyl group, carboxy alkyl group, N-hydroxy amide alkyl group, heteroaryl group.

The term “arylalkyl group” refers to a liner, branched or cycloalkyl which is linked to at least one aryl group. Preferable the number of carbon atoms in the chain or cyclic is 1-4. Exemplary arylalkyl group is —CH₂C₆H₅, —CH₂CH₂C₆H₅, —CH₂CH₂CH₂C₆H₅, —CH₂(CH₃)CH₂C₆H₅, —CH₂(CH₃)CH₂CH₂C₆H₅.

The term “heteroaryl alkyl group” refers to a liner, branched or cycloalkyl which is linked to at least one heteroaryl group. Preferable the number of carbon atoms in the chain or cyclic is 1-4. Exemplary heteroaryl alkyl group is

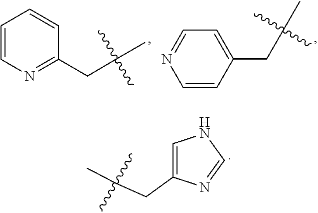 [SMILES: *Cc1ccccn1 ; *Cc1ccncc1 ; *Cc1c[nH]cn1]

The term “aryl alkoxy group” refers to an aromatic group, wherein one or several H atoms are replaced by alkoxy group. Exemplary phenyl-O—CH₂—, phenyl-O—(CH₂)₂—, phenyl-O—(CH₂)₃-, phenyl-O—(CH₂)₄—, phenyl-O—(CH₂)₅—.

The term “C₁-C₅ alkoxy group” refers to an oxygen atom attached to C₁-C₅ alkyl group. Exemplary C₁-C₅ alkoxy group is —OCH₃, —OCH₂CH₃, —OCH₂(CH₃)₂, —OCH₂CH₂CH₃.

The term “halogen” refers to F, Cl, Br or I.

The term “Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

The term “Cyano” refers to a substituent having a carbon atom joined to a nitrogen atom by a triple bond, e.g., —CN.

In some embodiments, R₁ is H, and R₂ is H.

In some embodiments, X is —OCH₃, —OCH₂CH₃, or —O(CH₃)₂.

In some embodiments, X is —NR₅R₆, R₅ is H, and
R₆ is H, C₀-C₅ hydroxyalkyl group, C₁-C₅ alkoxy group, optionally substituted heteroaryl alkyl group, optionally substituted aryl alkoxy group, optionally substituted aryl group, optionally substituted arylalkyl group, C₁-C₅ alkyl sulfonyl group or —(CH₂)n¹(OCH₂CH₂O)n²CH(R₈)CO(R₇),
R₇ is C₀-C₃ hydroxyalkyl group or —NHOH,
R₈ is H or optionally substituted arylalkyl group,
n¹ and n² independently are the number 0.

In some embodiments, R₆ is H, C₀-C₂ hydroxyalkyl group, C₁-C₃ alkoxy group, C₁-C₃ alkyl sulfonyl group, bipyridyl group, benzyl group, aryl alkoxy group, phenyl group which is optionally substituted with OH, carboxy or pyridyl group, or —CH(R₈)CO(R₇),
R₇ is OH or —NHOH,
R8 is H or benzyl group which is optionally substituted with OH, halogen, cyano group or nitro group.

In some embodiments, X is —NR₅R₆, R₅ is H, and
R₆ is H, OH, C₁ hydroxyalkyl group, C₂ hydroxyalkyl group, C₃ hydroxyalkyl group, C₄ hydroxyalkyl group, C₅ hydroxyalkyl group, C₁ alkoxy group, C₂ alkoxy group, C₃ alkoxy group, C₄ alkoxy group, C₅ alkoxy group, heteroaryl alkyl group optionally substituted with heteroaryl group, aryl methoxy group, aryl ethoxy group, aryl propoxy group, aryl butoxy group, aryl group optionally substituted with OH, carboxy or pyridyl group, benzyl group, aryl ethyl group, aryl propyl group, C₁ alkyl sulfonyl group, C₂ alkyl sulfonyl group, C₃ alkyl sulfonyl group, C₅ alkyl sulfonyl group, C₅ alkyl sulfonyl group or —(CH₂)n¹(OCH₂CH₂O)n²CH(R₈)CO(R₇),
R₇ is OH, C₁ hydroxyalkyl group, C₂ hydroxyalkyl group, C₃ hydroxyalkyl group or —NHOH,
R₈ is H or arylalkyl group which is optionally substituted with OH, halogen, cyano group or nitro group, wherein, halogen is selected from F, Cl, Br or I,
n¹ and n² independently are the number 0.

In some embodiments, X is —NR₅R₆, R₈ is H, and
R₆ is H, OH, —CH₂OH, —(CH₂)₂OH, —CH₃, —CH₂CH₃, —CH₂COOH, —(CH₂)₂COOH, —(CH₂)₃COOH, —(CH₂)₄COOH, —(CH₂)₅COOH, —OCH₃, —OCH₂CH₃, —CH₂CONHOH, —OC(C₆H₅)₃, —(CH₂)₃S(O)₂OH,

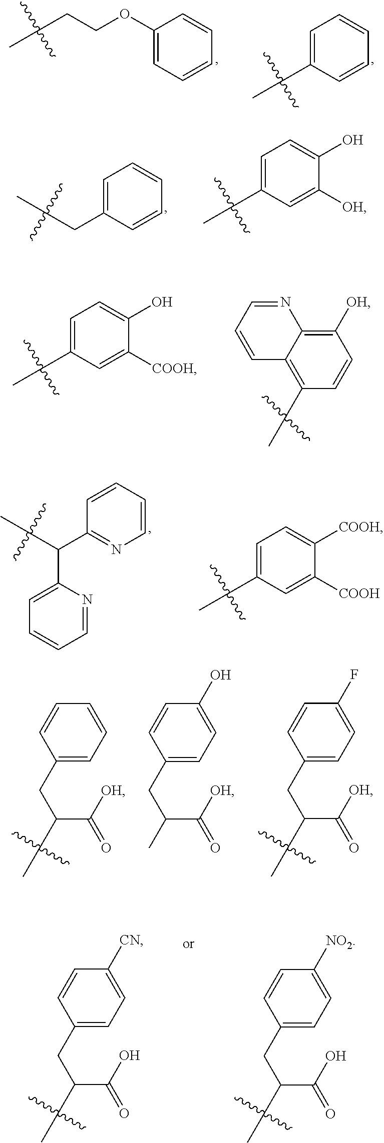

In some embodiments, X is —NR₅R₆, R₈ is H, and
R₆ is H, OH, —CH₂COOH, —CH₂CONHOH, —OC(C₆H₅)₃,

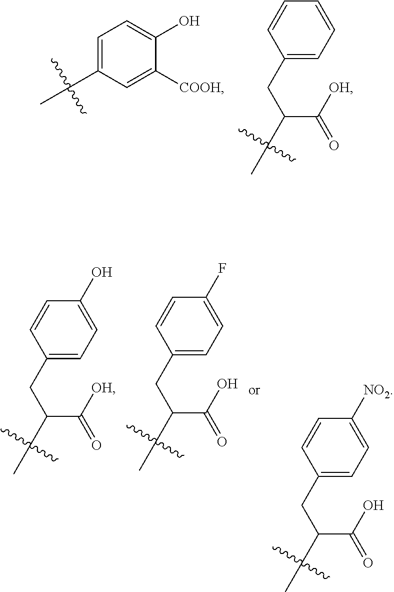 [SMILES: *c1ccc(O)c(C(=O)O)c1 ; *C(Cc1ccccc1)C(=O)O ; *C(Cc1ccc(O)cc1)C(=O)O ; *C(Cc1ccc(F)cc1)C(=O)O ; *C(Cc1ccc([N+](=O)[O-])cc1)C(=O)O]

In some embodiments, X is —NR₅R₆, R₅ is H, and R₆ is OH, —CH₂COOH, —(CH₂)₂COOH, —(CH₂)₃COOH, —(CH₂)₄COOH, —(CH₂)₅COOH, —OCH₃, —OCH₂CH₃, or —OC(C₆H₅)₃.

In some embodiments, X is —NR₅R₆, R₅ is OH,
R₆ is C₁-C₅ alkyl group, optionally substituted heteroaryl alkyl group, optionally substituted arylalkyl group, optionally substituted aryl group, or —(CH₂)n¹(OCH₂CH₂O)n²CH(R₈)CO(R₇),
R7 is C₀-C₅ hydroxyalkyl group,
R8 is H,
n¹ and n² independently are the number 0, 1, 2, 3, 4.

In some embodiments, R₆ is C₁-C₃ alkyl group, heteroaryl alkyl group which comprises a heteroatom N, optionally substituted benzyl group, optionally substituted phenyl group, or —CH(R₈)CO(R₇),
R₇ is C₀-C₃ hydroxyalkyl group,
R₈ is H.

In some embodiments, R₆ is C₁ alkyl group, C₂ alkyl group, C₃ alkyl group, C₄ alkyl group, C₅ alkyl group, heteroaryl methyl group, heteroaryl ethyl group, heteroaryl propyl group, benzyl group, aryl ethyl group, aryl propyl group, or —CH(R₈)CO(R₇),
R₇ is hydroxy, C₁ hydroxyalkyl group, C₂ hydroxyalkyl group, C₃ hydroxyalkyl group, C₄ hydroxyalkyl group or C₅ hydroxyalkyl group,
R₈ is H.

In some embodiments, R₆ is —CH₃, —CH₂COCH₃, —CH₂COOH,

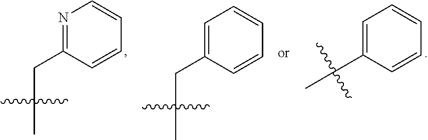 [SMILES: *Cc1ccccn1 ; *Cc1ccccc1 ; *c1ccccc1]

In some embodiments, R₆ is —CH₂COOH.

In some embodiments, X is —NR₅R₆,
R₅ and R₆ independently are C₁-C₅ alkyl group, C₀-C₅ hydroxyalkyl group, optionally substituted heteroaryl alkyl group or —(CH₂)n¹(OCH₂CH₂O)n²CH(R₈)CO(R₇),
R₇ is C₀₋₅ hydroxyalkyl group or —NHOH,
R₈ is H,
n¹ and n² independently are the number 0, 1, 2, 3, 4.

In some embodiments, X is —NR₅R₆,
R₅ and R₆ independently are C₁ alkyl group, C₂ alkyl group, C₃ alkyl group, C₄ alkyl group, C₅ alkyl group, OH, C₁ hydroxyalkyl group, C₂ hydroxyalkyl group, C₃ hydroxyalkyl group, C₄ hydroxyalkyl group, C₅ hydroxyalkyl group, heteroaryl methyl group, heteroaryl ethyl group, heteroaryl propyl group or —(CH₂)n¹(OCH₂CH₂O)n²CH(R₈)CO(R₇),
R₇ is hydroxy, C₁ hydroxyalkyl group, C₂ hydroxyalkyl group, C₃ hydroxyalkyl group, C₄ hydroxyalkyl group, C₅ hydroxyalkyl group or —NHOH,
R₈ is H,
n¹ and n² independently are the number 0, 1, 2, 3, 4.

In some embodiments, X is —NR₅R₆, R₅ and R₆ independently are methyl, ethyl group, —(CH₂)₂OH, —CH₂COOH, —CH₂CONHOH or

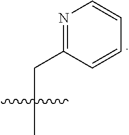 [SMILES: *Cc1ccccn1]

In some embodiments, Y is

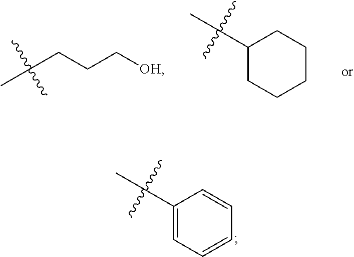 [SMILES: *CCCO ; *C1CCCCC1 ; *c1ccccc1]

### Z is

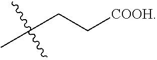 [SMILES: *CCC(=O)O]

In some embodiments, the reductant is selected from the group consisting of

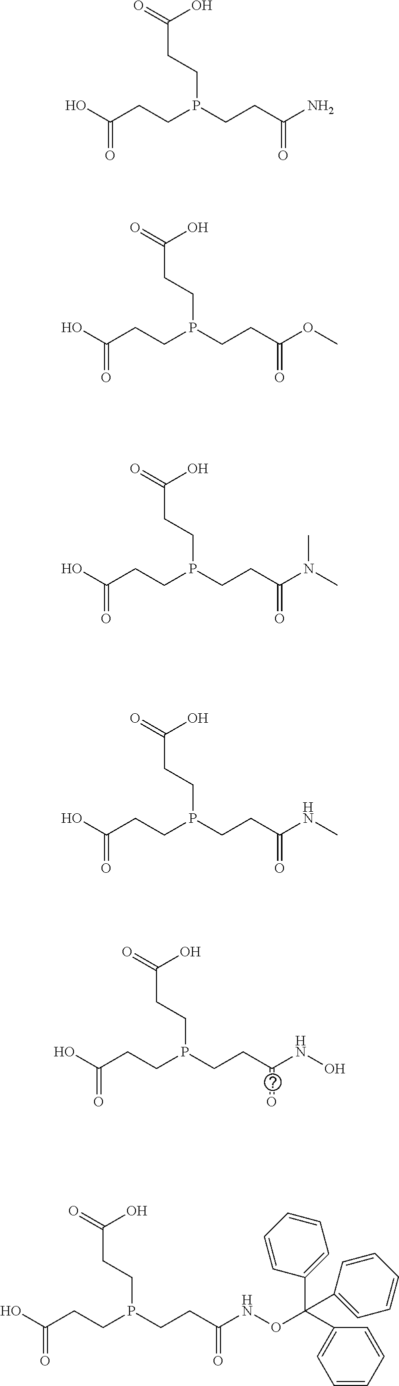 [SMILES: NC(=O)CCP(CCC(=O)O)CCC(=O)O ; COC(=O)CCP(CCC(=O)O)CCC(=O)O ; CN(C)C(=O)CCP(CCC(=O)O)CCC(=O)O ; CNC(=O)CCP(CCC(=O)O)CCC(=O)O ; O=C(O)CCP(CCC(=O)O)CCC(=O)NO ; O=C(O)CCP(CCC(=O)O)CCC(=O)NOC(c1ccccc1)(c1ccccc1)c1ccccc1]

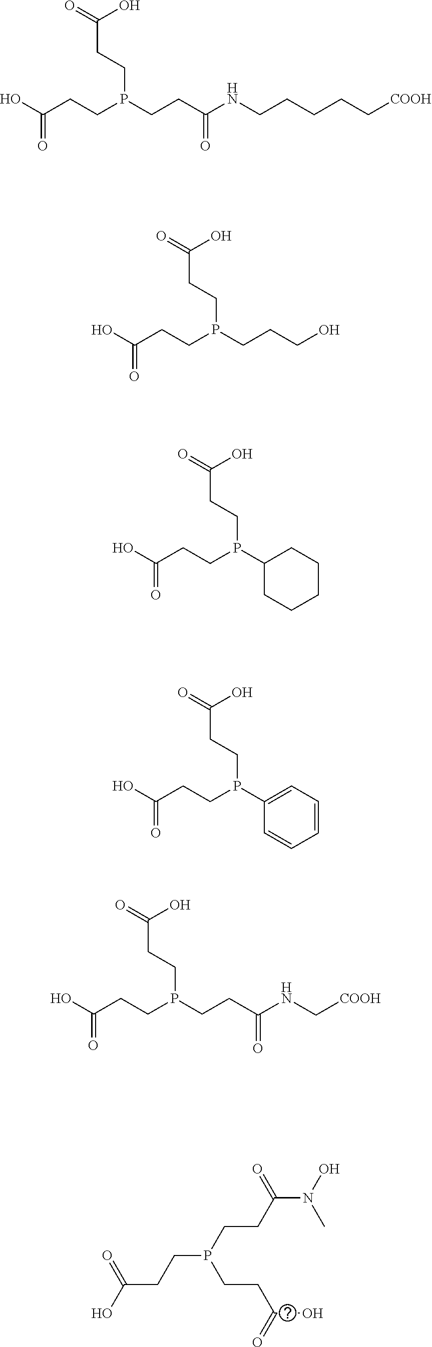 [SMILES: O=C(O)CCCCCNC(=O)CCP(CCC(=O)O)CCC(=O)O ; O=C(O)CCP(CCCO)CCC(=O)O ; O=C(O)CCP(CCC(=O)O)C1CCCCC1 ; O=C(O)CCP(CCC(=O)O)c1ccccc1 ; O=C(O)CCP(CCC(=O)O)CCC(=O)NCC(=O)O ; CN(O)C(=O)CCP(CCC(=O)O)CCC(=O)O]

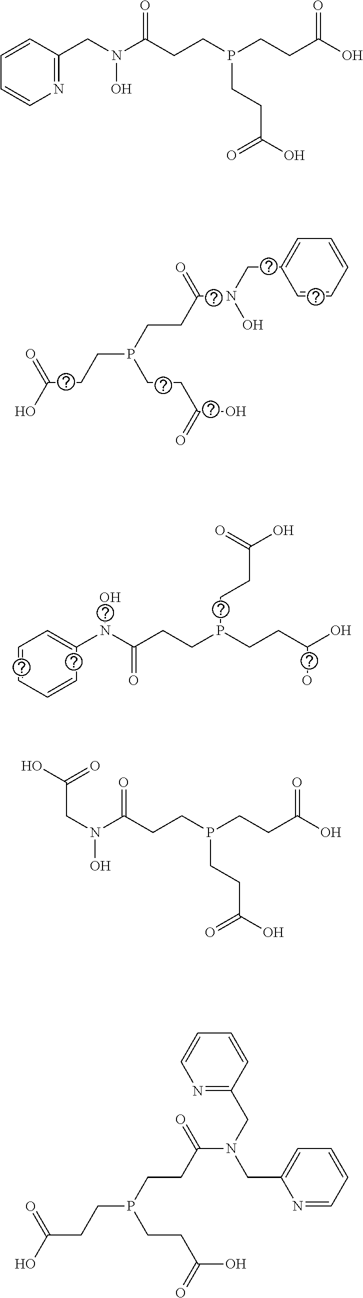 [SMILES: O=C(O)CCP(CCC(=O)O)CCC(=O)N(O)Cc1ccccn1 ; O=C(O)CCP(CCC(=O)O)CCC(=O)N(O)Cc1ccccc1 ; O=C(O)CCP(CCC(=O)N(O)c1ccccc1)CCC(O)O ; O=C(O)CCP(CCC(=O)O)CCC(=O)N(O)CC(=O)O ; O=C(O)CCP(CCC(=O)O)CCC(=O)N(Cc1ccccn1)Cc1ccccn1]

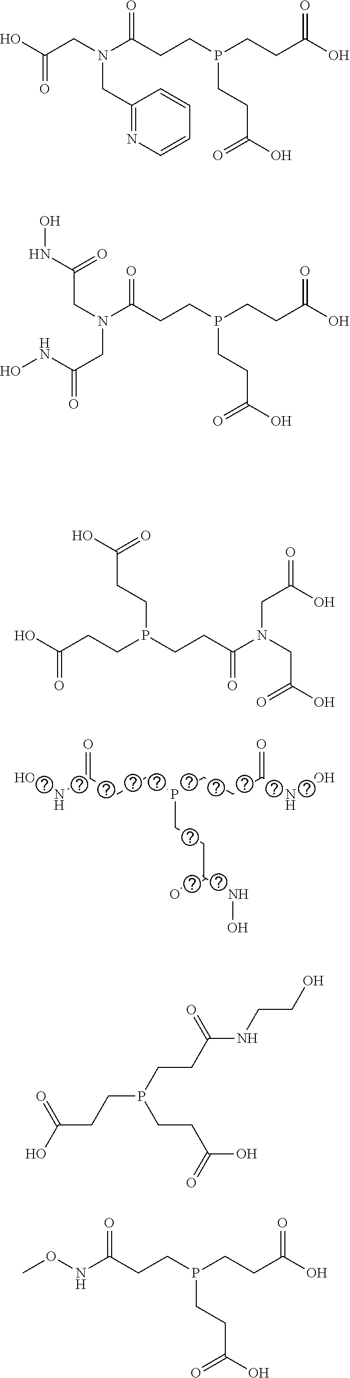

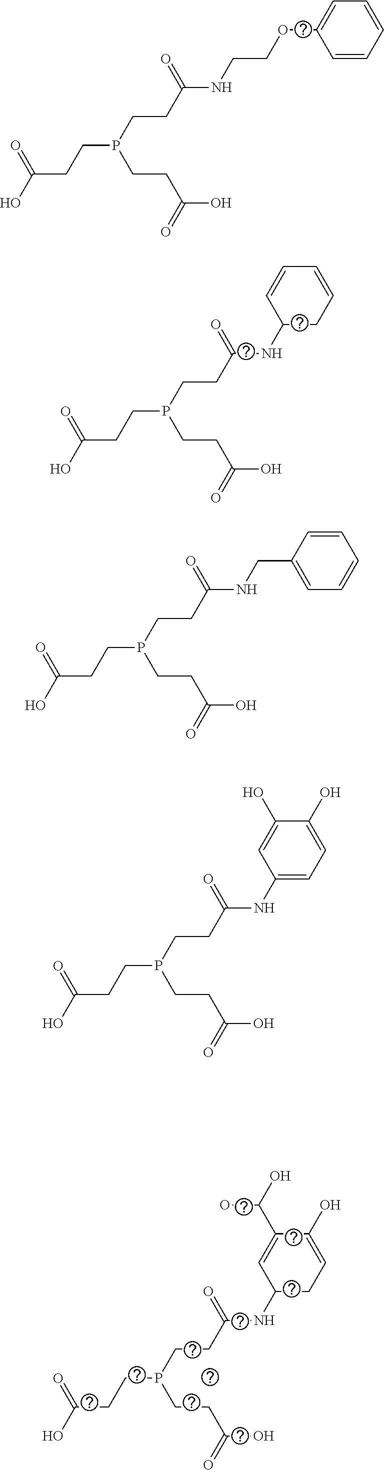 [SMILES: O=C(O)CCP(CCC(=O)O)CCC(=O)NCCOc1ccccc1 ; O=C(O)CCP(CCC(=O)O)CCC(=O)NC1C=CC=CC1 ; O=C(O)CCP(CCC(=O)O)CCC(=O)NCc1ccccc1 ; O=C(O)CCP(CCC(=O)O)CCC(=O)Nc1ccc(O)c(O)c1 ; O=C(O)CCP(CCC(=O)O)CCC(=O)NC1C=C(C(O)O)C(O)=CC1]

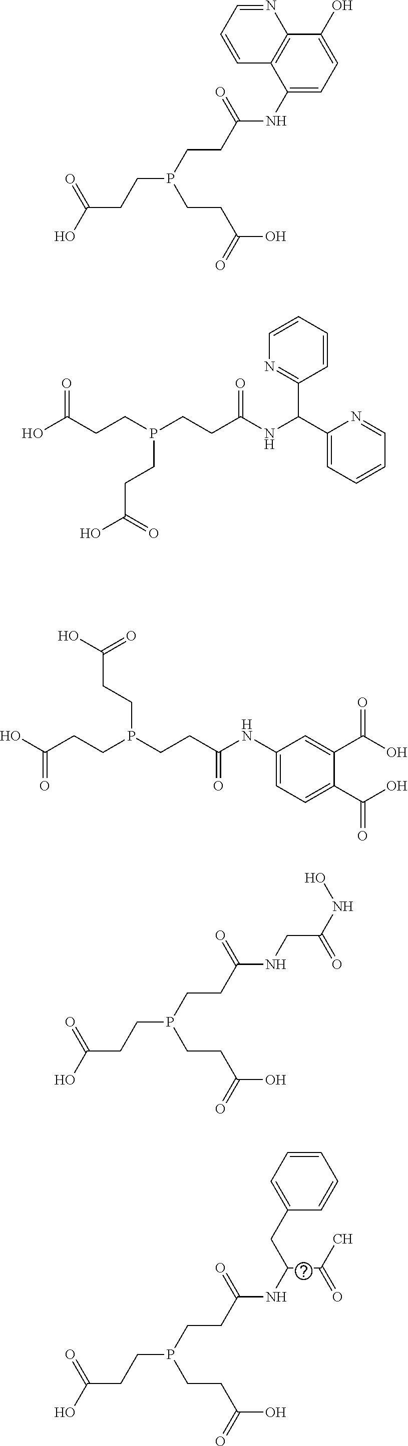 [SMILES: O=C(O)CCP(CCC(=O)O)CCC(=O)Nc1ccc(O)c2ncccc12 ; O=C(O)CCP(CCC(=O)O)CCC(=O)NC(c1ccccn1)c1ccccn1 ; O=C(O)CCP(CCC(=O)O)CCC(=O)Nc1ccc(C(=O)O)c(C(=O)O)c1 ; O=C(O)CCP(CCC(=O)O)CCC(=O)NCC(=O)NO ; CC(=O)C(Cc1ccccc1)NC(=O)CCP(CCC(=O)O)CCC(=O)O]

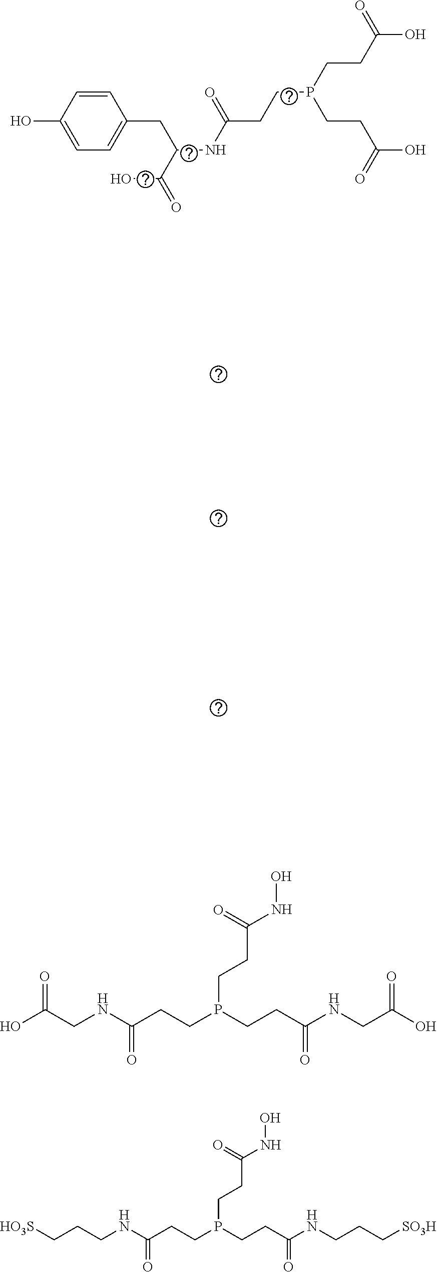 [SMILES: O=C(O)CCP(CCC(=O)O)CCC(=O)NC(Cc1ccc(O)cc1)C(=O)O ; O=C(O)CNC(=O)CCP(CCC(=O)NO)CCC(=O)NCC(=O)O ; O=C(CCP(CCC(=O)NCCCS(=O)(=O)O)CCC(=O)NCCCS(=O)(=O)O)NO]

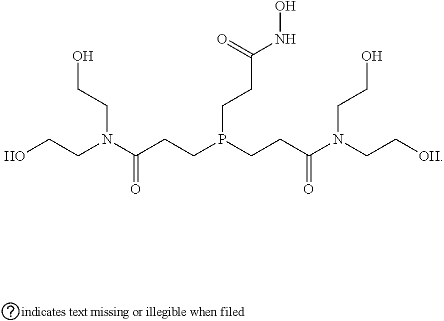 [SMILES: O=C(CCP(CCC(=O)N(CCO)CCO)CCC(=O)N(CCO)CCO)NO]

The disclosure provides a composition including a reductant described above and transition metal ions.

In some embodiments, the transition metal ions are Zn²⁺, Cd²⁺, Hg²⁺, Ni²⁺, Co²⁺ or the combination thereof. In some embodiments, the transition metal ions are Zn²⁺.

In some embodiments, the molar ratio of the transition metal ions and the first reductant described above is 0.05:1 to 40:1, 0.25:1 to 30:1, 0.25:1 to 15:1, 0.1:1 to 10:1, 0.25:1 to 9:1, 0.2:1 to 7.5:1, 0.2:1 to 6:1, 0.2:1 to 5:1, 0.25:1 to 4:1, 0.5:1 to 7.5:1, 1:1 to 7.5:1, or 2:1 to 4:1.

The reductant having formula (I) described above could be prepared as the following steps:
at least one X′ is connected to a compound of formula II by introducing a condensation reagent under an inert atmosphere,

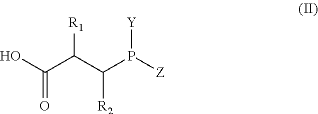

R₁ is H, —NH₂, —C(O)(R₃R₄), optionally substituted C₁-C₅ alkyl group, optionally substituted C₁-C₅ hydroxyalkyl group, or optionally substituted aryl group;
R₃ is N, NH or O;
R₄ is H, optionally substituted C₁-C₅ alkyl group, optionally substituted C₁-C₅ hydroxyalkyl group, or optionally substituted aryl group;
R₂ is H, optionally substituted C₁-C₅ alkyl group, or optionally substituted C₁-C₅ hydroxyalkyl group;
X′ is optionally substituted C₁-C₅ alkyl alcohol or NR₅R₆,
R₅ and R₆ independently are H, C₀-C₅ hydroxyalkyl group, optionally substituted C₁-C₈alkyl group, optionally substituted C₂-C₅ carboxy alkyl group, optionally substituted C₁-C₅ alkoxy group, optionally substituted heteroaryl alkyl group, optionally substituted aryl alkoxy group, optionally substituted arylalkyl group, optionally substituted aryl group, C₁-C₅ alkyl sulfonyl group, —(CH₂)n¹(OCH₂CH₂O)n²CH(R₈)CO(R₇).
R₇ is C₀-C₅ hydroxyalkyl group, —NHOH,
R₈ is H, optionally substituted arylalkyl group,
n¹ and n² independently are the number 0, 1, 2, 3, 4,
Y is the same as X, or Y is an ester or amide of X,
Z is the same as X or Y, or
Y and Z independently are selected from the group consisting of

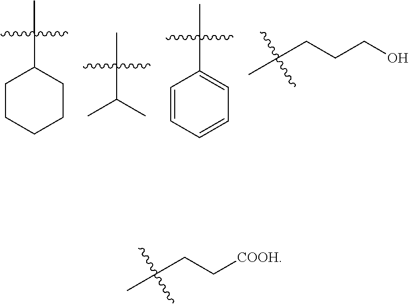

The term “condensation reagent” refers to a condensation reaction reagent, which helps two mol ecules (functional groups) combine covalently to form one single molecule. Condensation reagent inc ludes, but not limited to 1-Hydroxybenzotriazole (HOBT), O-Benzotriazole-N,N,N′,N′-tetramethyl-uronium-hexafluorophosphate (HBTU), and O-(Benzotriazol-1-yl)-N,N,N′,N′-tetramethyluronium tetrafluoroborate (TBTU).

The term “inert atmosphere” refers to the chemically inactive atmosphere, such as nitrogen, carbon dioxide, helium.

In some embodiments, the compound of formula II is

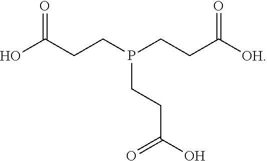 [SMILES: O=C(O)CCP(CCC(=O)O)CCC(=O)O]

In some embodiments, characterized in that, the X′ is 2-phenoxy-ethylamine, Phenylamine, Benzylamine, 4-Aminobenzene-1,2-diol, 5-Amino-2-hydroxybenzoic acid, Bis(pyridin-2-ylmethyl)amine, 5-Amino-8-hydroxyquinoline, Bis(pyridin-2-yl) methanamine, 4-Aminophthalic acid, tert-Butyl L-tyrosinate, DL-3-(4-Fluorophenyl)alanine, DL-4-Cyanophenylalanine, DL-4-nitro-phenylalanine, N-Benzylhydroxylamine hydrochloride, N-Phenylhydroxylamine,

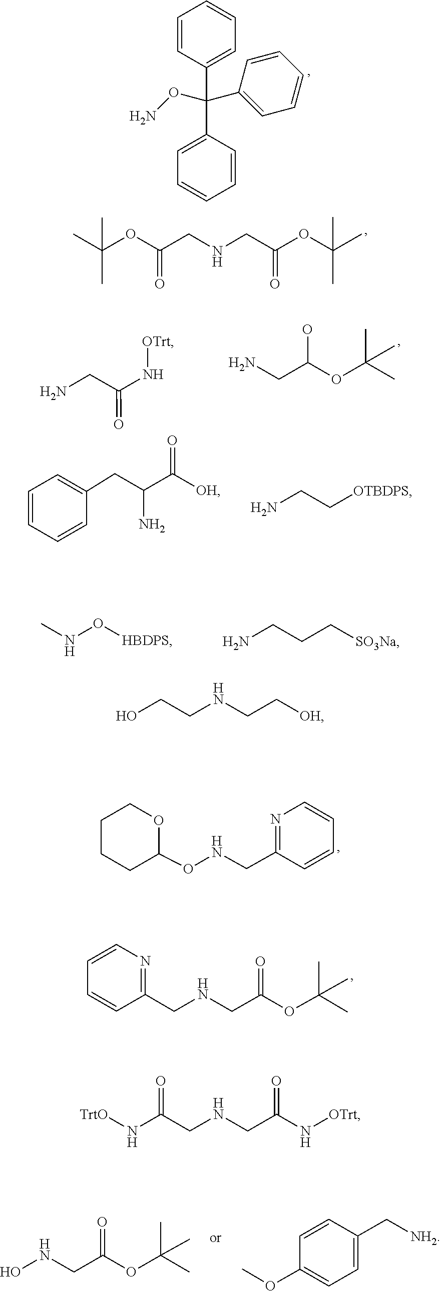

### Use of Reductant

The reductant having formula (I) provided above has reducibility and could reduce the disulfide bond of an antibody, thus the reductant could be used to modify protein or antibody.

As used herein, the term “disulfide bond” refers to a covalent bond with the structure R—S—S—R′.

The amino acid cysteine includes a thiol group that can form a disulfide bond with a second thiol group, for example from another cysteine residue. The disulfide bond can be formed between the thiol groups of two cysteine residues residing respectively on the two poly-peptide chains, thereby forming an interchain bridge or interchain bond.

In some embodiments, the reductant having formula (I) could reduce the interchain disulfide bonds of an antibody.

In some embodiments, the reductant having formula (I) could selectively reduce three of the interchain disulfide bonds of the antibody.

In some embodiments, the reductant having formula (I) could selectively reduce the interchain disulfide bonds, thus the antibody is selectively modified.

In some embodiments, provided herein is the use of the reductant having formula (I) provided above and the composition described above in reducing the interchain bonds of an antibody, optionally, provided herein is the use of the reductant having formula (I) provided above and the composition described above in reducing three of the interchain bonds of an antibody.

In some embodiments, the use of the reductant having formula (I) in the preparation of an antibody with site-specific modification, optionally, the antibody with site-specific modification is antibody drug conjugate (ADCs).

A mixture of antibody-drug conjugates will be generated by the conventional conjugation processes or the bio-conjugation process of the present disclosure. In general, one antibody molecule belonging to IgG1 or IgG4 subclass has 4 inter-chain disulfide bonds, each of which is formed with two —SH groups. The antibody molecule can be subjected to partial or complete reduction of one or more interchain disulfide bonds to form 2n (n is an integer selected from 1, 2, 3 or 4) reactive —SH groups, and thus, the number of drugs (or payloads) coupling to a single antibody molecule is 1, 2, 3, 4, 5, 6 7, or 8. In accordance with the number of drugs coupling to a single antibody molecule, the different conjugates containing different number of drug molecules are denominated as D0, D2, D4, D6, D8, D3, Dt, D6+D1, D6+D2, D3+D1, D3+D2, D0+D1, D0+D2, D1+D6, D1+D2, D1+D4 or D2+D4. And thus, the “homogeneity” of antibody-drug conjugates is used to describe the property of dominance of one specific type of antibody-drug conjugate (i.e., one type selected from D0, D2, D4, D6, D8, D3, D1, D6+D1, D6+D2, D3+D1, D3+D2, D0+D1, D0+D2, D1+D6, D1+D2, D1+D4 or D2+D4 conjugates) in one given mixture of antibody-drug conjugates.

Drug to Antibody Ratio (DAR) of ADC is the average number of drugs linked to each antibody. DAR is a key property used to measures the quality of ADC because it can significantly affect ADC efficacy. The DAR distribution (D0, D2, D4, D6, D8) could reflect the homogeneity of the ADC.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, the average drug loading for an antibody-drug conjugate ranges from 1 to about 8; from about 2 to about 6; or from about 3 to about 5.

As used herein, the term “DO” or “the ADC with DO” refers to the ADC in which the average number of drugs coupling to a single antibody molecule is about zero.

As used herein, the term “D2” or “the ADC with D2” refers to DAR about 2, it means about two drug molecules (e.g., 1.5, 2.0, 2.5 molecules) are coupled to one single antibody molecule on average.

Drug molecules may be coupled to —SH groups generated by reduction of disulfide bond between heavy and light chains or heavy and heavy chains via linkers.

As used herein, the term “D4” or “the ADC with D4” refers to the ADC in which about four drug molecules (e.g., 3.5, 4.0, 4.5 molecules) are coupled to one single antibody molecule on average, where the drug molecules may be coupled to four —SH groups generated by reduction of two interchain disulfide bonds or intrachain disulfide bonds.

As used herein, the term “D6” or “the ADC with D6” refers to the ADC in which about six drug molecules (e.g., 5.5, 6.0, 6.5 molecules) are coupled to one single antibody molecule on average, where the drug molecules may be coupled to six —SH groups generated by reduction of three disulfide bond.

As used herein, the term “D8” or “the ADC with D8” refers to the ADC in which about eight drug molecules (e.g., 7.5, 8.0, 8.5 molecules) are coupled to one single antibody molecule on average, where the drug molecules may be coupled to eight-SH groups generated by reduction of four disulfide bond.

As used herein, the term “D1” or “the ADC with D1” refers to the ADC in which one of the first thio-bridging group bearing the first linker-payload re-bridges two thiol groups of one single antibody molecule.

As used herein, the term “D3” or “the ADC with D3” refers to the ADC in which three of the first thio-bridging group bearing the first linker-payload re-bridges six thiol groups of one single antibody molecule.

As used herein, the term “D6+D1” or “the bi-payload ADC with D6+D1” refers to the ADC in which six of the first linker-payloads and one of the second thio-bridging groups bearing the second linker-payload are coupled to one single antibody molecule.

As used herein, the term “D6+D2’ or “the bi-payload ADC with D6+D2” refers to the ADC in which six of the first linker-payloads and two of the second linker-payloads are coupled to one single antibody molecule.

As used herein, the term “D3+D1” or “the bi-payload ADC with D3+D1” refers to the ADC in which three of the first thio-bridging group bearing the first linker-payload and one of the second thio-bridging group bearing the second linker-payload re-bridge eight thiol groups of one single antibody molecule.

As used herein, the term “D3+D2” or “the bi-payload ADC with D3+D2” refers to the ADC in which three of the first thio-bridging group bearing the first linker-payload re-bridge six thiol groups and two of the second linker-payloads are coupled to one single antibody molecule.

As used herein, the term “D0+D2” or “the ADC with D0+D2” refers to the ADC in which one, two or three of the first thio-bridging group re-bridge six thiol groups and two of the second linker-payloads are coupled to one single antibody molecule, or refers to the ADC in which two, four or six of the end capping reagents and two of the second linker-payloads are coupled to one single antibody molecule.

As used herein, the term “D0+D1” or “the ADC with D0+D1” refers to the ADC in which three of the first thio-bridging group re-bridges six thiol groups and one of the second thio-bridging group bearing the linker-payload re-bridge two thiol groups of one single antibody molecule, or refers to the ADC in which six of the end capping reagents react with six thiol groups and one of the second thio-bridging group bearing the linker-payload re-bridge two thiol groups of one single antibody molecule.

As used herein, the term “D1+D6” or “the bi-payload ADC with D1+D6” refers to the ADC in which one of the first t thio-bridging group bearing the first linker-payload re-bridging two thiol groups and six of the second linker-payloads are coupled to one single antibody molecule, wherein, the first linker-payload and the second linker-payload may be same or different.

As used herein, the term “D1+D2” or “the bi-payload ADC with D1+D2” refers to the ADC in which one of the first thio-bridging group bearing the first linker-payload re-bridging two thiol groups and two of the second linker-payloads are coupled to one single antibody molecule, wherein, the first linker-payload and the second linker-payload may be same or different.

As used herein, the term “D1+D4” or “the bi-payload ADC with D1+D4” refers to the ADC in which one of the first thio-bridging group bearing the first linker-payload re-bridging two thiol groups and four of the second linker-payloads are coupled to one single antibody molecule, wherein, the first linker-payload and the second linker-payload may be same or different.

As used herein, the term “D2+D4” or “the bi-payload ADC with D2+D4” refers to the ADC in which two of the first linker-payloads and four of the second linker-payloads are coupled to one single antibody molecule.

As used herein, the term “about” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” when preceding a numerical value indicates the value plus or minus a range of 50%, 30%, 15%, 10%, 5%, or 1%.

In some embodiments, by conjugating two different linker-payloads to a single antibody to form a bi-payload ADC. According to the DAR value of each linker-payload, the bi-payload ADC may be with two DAR values, such as D6 for first linker-payload and D2 for second linker-payload. The DAR value of di-payload ADC in present disclosure is referred to as DN+DM, of which N denotes the average number of the first linker-payload coupled to one single antibody molecule on average, and M denotes the average number of the second linker-payload coupled to one single antibody molecule on average.

In some embodiments, the reductant having formula (I) provided above or the composition provided above could be used to prepare ADC with improved homogeneity.

In some embodiments, the disclosure provides the use of reductant having formula (I) or the composition provided above in the preparation of ADC, optionally, in the preparation of the ADC with D2, the ADC with D4, the ADC with D1, the ADC with D6, the ADC with D3, the ADC with D1+D6, the ADC with D1+D2, the ADC with D1+D4, the ADC with D2+D4, the ADC with D6+D2, the ADC with D6+D1, the ADC with D3+D1, the ADC with D3+D2, the ADC with D0+D6, or the ADC with D0+D2.

In some embodiments, the ADC includes D6 in a content at least up to 50% of the total weight of D0, D2, D4, D6 and D8 combined. In some embodiments, the ADC includes D6 in a content up to 60%, 65%, 70%, 75%, 80%, 81%, 82%, 83%, 84%, 85%, 86%, 87%, 88%, 89%, 90%, 91%, 92%, 93%, 94%, 95% or 96% of the total weight of D0, D2, D4, D6 and D8 combined.

In some embodiments, the ADC includes D2 in a content at least up to 70% of the total weight of D0, D2, D4, D6 and D8 combined. In some embodiments, the ADC includes D6 in a content up to 75%, 80%, 81%, 82%, 83%, 84%, 85%, 86%, 87%, 88%, 89%, 90%, 91%, 92%, 93%, 94%, or 95% of the total weight of D0, D2, D4, D6 and D8 combined.

In some embodiments, the homogeneity of the ADC with D3 is up to 82%, 83% or 85%.

In some embodiments, the homogeneity of the ADC with D6+D2 is up to 80%, 81%, 82%, 83%, 84%, 85% or 86%.

In some embodiments, the homogeneity of the ADC with D6+D1 is up to 80%, 810%, 82%, 83%, 84%, 85%, 86%, 87%, 88%, 89%, 90%, 91% or 92%.

In some embodiments, the homogeneity of the ADC with D0+D2 is up to 65%, 67%, 69%, 70%, 71% or 73%.

In some embodiments, the homogeneity of the ADC with D0+D1 is up to 70%, 71%, 73%, 75%, 77%, 79%, 80% or 83%.

In some embodiments, the homogeneity of the ADC with D1 is up to 77%, 79%, 80%, 83% or 85%.

In some embodiments, the homogeneity of the ADC with D1+D6 is up to 80%, 81%, 82%, 83%, 84%, 85%, 86%, 87%, 88%, 89% or 90%.

In some embodiments, the homogeneity of the ADC with D1+D2 is up to 65%, 67%, 69%, 70%, 71%, 73%, 75% or 77%.

In some embodiments, the homogeneity of the ADC with D1+D4 is up to 80%, 81%, 82%, 83%, 84%, 85%, 86%, 87%, 88%, 89% or 90%.

In some embodiments, the homogeneity of the ADC with D2+D4 is up to 60%, 77%, 79%, 80%, 83% or 85%.

Antibody with Site-Specific Modification and Preparation Thereof

The present disclosure also provides a method of preparing an antibody with site-specific modification by using the reductant of formula (I) and the modification antibody thereof. Optionally, the present disclosure provides a method of preparing ADCs with improved homogeneity by using the reductant of formula (I) or a salt, solvate, stereoisomer thereof.

In some embodiments, the method of preparing an antibody with site-specific modification includes the following steps:

(A1) incubating a reductant of formula (I) or a salt, solvate, stereoisomer thereof as a first reductant and the transition metal ions in the presence of an antibody in a buffer system to selectively the reduce interchain disulfide bonds within the antibody to afford the antibody bearing reduced thiol groups.

In some embodiments, two interchain disulfide bonds in Fab region of the antibody and one interchain disulfide bonds in hinge region of the antibody are reduced in step (A1).

In some embodiments, the method of preparing the antibody with site-specific modification further includes the step:

(A2) introducing oxidant to selectively re-oxidize the reduced thiol groups resulted from step (A1).

In some embodiments, the oxidant in step (A2) re-oxidizes the reduced thiol groups in Fab region of the antibody. Optionally, four of the reduced thiol groups are re-oxidized to form two disulfide bonds.

In some embodiments, the resulting product of step (A2) also performs purification after oxidation. The purification could conduct by, include but not limited to, a desalting column, ultrafiltration (UF) and diafiltration (DF).

In some embodiments, the method of preparing the antibody with site-specific modification further includes the step,

(A3) incubating a second reductant in a buffer system to reduce the interchain disulfide bonds resulted from step (A2).

In some embodiments, the second reductant reduce the interchain disulfide bonds of the antibody in step (A3), optionally, one interchain disulfide bond in hinge region is reduced.

In some embodiments, the method of preparing the antibody with site-specific modification further comprising the following step of,

(B1) introducing metal chelators and first payload units to react with the reduced thiol groups resulted from step (A1), step (A2) or step (A3), wherein, the first payload unit is an end capping reagent, a first linker-payload or a first thio-bridging reagent, optionally, the first thio-bridging reagent bears the first linker-payload or reactive groups.

In some embodiments, when the first payload units are the first thio-bridging reagent bearing reactive groups, the step (B1) further comprising step of
incubating the metal chelators and the first linker-payloads in the buffer system to react with the reactive groups of the first thio-bridging reagent bearing reactive groups.

In some embodiments, the method of preparing the antibody with site-specific modification further includes the steps:

(B2) incubating a second reductant in a buffer system to reduce interchain disulfide bonds resulted from step (B1), optionally, introducing the transition metal ions; and

(B3) introducing second payload units to react with the reduced thiol groups resulted from step (B2), optionally, introducing the metal chelators, wherein, the second payload unit is a second linker-payload or a second thio-bridging reagent, optionally, the second thio-bridging reagent bears the second linker-payload or reactive groups.

In some embodiments, without the transition metal ions, the second reductant reduces all the interchain disulfide bonds of the antibody in step (B2). In some embodiments, with step (A1), (B1) and (B2), one interchain disulfide bond in the hinge region of the antibody is reduced. In some embodiments, with step (A1), (A2), (B1) and (B2), three interchain disulfide bonds are reduced. In some embodiments, with step(A1), (A2), (A3), (B1) and (B2), two interchain disulfide bonds are reduced.

In some embodiments, with the transition metal ions, the second reductant selective reduces the interchain disulfide bonds of the antibody, optionally, one or two interchain disulfide bonds are reduced. In some embodiments, with step (A1), (A2), (B1) and (B2), the second reductant and the transition metal ions selective reduce one or two interchain disulfide bonds of the antibody.

In some embodiments, when the second payload units are the second thio-bridging reagent bearing reactive groups, the step (B3) further comprising step of incubating the second linker-payloads in the buffer system to react with the reactive groups of the second thio-bridging reagent bearing reactive groups, optionally, introducing the metal chelators.

In some embodiments, when introducing the transition metal ions in step (B2), introducing the metal chelators to trap the excess transition metal ions in step (B3).

In some embodiments, the molar ratio of the reductant of formula (I) or the second reductant and the antibody in step (A1), (A3) and (B2) independently is 1:1 to 20:1, 1:1 to 10:1, 1:1 to 8:1, 1:1 to 5:1, 2:1 to 5:1, 3:1 to 5:1, 1:1 to 3:1, 1:1 to 2:1, 2:1 to 4:1, or 3:1 to 4:1.

In some embodiments, the molar ratio of the first reductant and the antibody in step (A1) is 2.8:1 to 13:1, optionally, the molar ratio of the first reductant and the antibody is 3.5:1 to 5:1, 4:1 to 10:1 or 5:1 to 13:1, 3:1 to 5:1, 4:1 to 5:1, or 3.8:1 to 4.6:1. In some embodiments, the molar ratio of the first reductant and the antibody in step (A1) is 2.8:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1.

In some embodiments, the incubation temperature in step (A1), (A3) and (B2) independently is 0° C. to 37° C., 0° C. to 25° C., 0° C. to 15° C., 0° C. to 10° C., or 0° C. to 5° C.

In some embodiments, the incubation temperature in step (A1) is 37° C., 35° C., 33° C., 30° C., 28° C., 24° C., 20° C., 18° C., 15° C., 13° C., 10° C., 8° C., 4° C. or 0° C.

In some embodiments, the incubation time in step (A1) is 2 h to 24 h, 14 h to 24 h, 16 h to 20 h, or 16 h to 18 h. In some embodiments, the incubation time in step (A1) is 2 h, 4 h, 6 h, 8 h, 10 h, 12 h, 14 h, 16 h, 18 h, 20 h, 22 h or 24 h.

In some embodiments, in step (A1), the molar ratio of the first reductant and the antibody is 2.8:1 to 3:1, the incubation time is 10 h to 24 h. In some embodiments, the incubation time in step (A1), the molar ratio of the first reductant and the antibody is 2.8:1, 2.9:1 or 3:1, the incubation time is 10 h, 121 h, 16 h, 18 h, 20 h, 22 h or 24 h.

In some embodiments, the incubation time in step (A1) is shortened with increasing the molar ratio of the first reductant and the antibody. In some embodiments, in step (A1), the molar ratio of the first reductant and the antibody is 4:1 to 20:1, the incubation time is 1 h to 24 h. In some embodiments, in step (A1), the molar ratio of the first reductant and the antibody is 4:1 to 10:1, the incubation time is 2 h to 16 h. In some embodiments, in step (A1), the molar ratio of the first reductant and the antibody is 4:1, 4.5:1, 5:1, 5:1, 7:1, 8:1, 9:1 or 10:1, the incubation time is 1 h, 2 h, 4 h, 5 h, 6 h, 7 h, 8 h, 9 h, 10, 11 h or 12 h.

In some embodiments, in step (A1), the molar ratio of the first reductant and the antibody is 5:1 to 20:1, the incubation time is 3 h to 24 h. In some embodiments, in step (A1), the molar ratio of the first reductant and the antibody is 6:1 to 13:1, the incubation time is 4 h to 16 h. In some embodiments, in step (A1), the molar ratio of the first reductant and the antibody is 4.5:1, 5:1, 5:1, 7:1, 8:1, 9:1, 10:1, 11:1, 12:1 or 13:1, the incubation time is 4 h, 5 h, 6 h, 7 h, 8 h, 9 h, 10, 11 h, 12 h, 13 h, 14 h, 15 h or 16 h.

In some embodiments, the molar ratio of the transition metal ions and the first reductant in step (A1) is 0.05:1 to 40:1, 0.08:1 to 30:1, 0.1:1 to 20:1, 0.2:1 to 8:1, or 0.25:1 to 7.5:1.

In some embodiments, the molar ratio of the transition metal ions and the first reductant in step (A1) is 0.1:1 to 20:1, 0.2:1 to 20:1, 0.1:1 to 10:1, 0.1:1 to 8:1, 0.2:1 to 8:1, 0.25:1 to 7.5:1, or 0.4:1 to 1:1.

In some embodiments, the molar ratio of the transition metal ions and the first reductant in step (A1) is 0.08:1, 0.2:1, 0.5:1, 0.8:1, 1:1, 2:1, 4:1, 8:1, 10:1, 12:1, 14:1, 16:1, 18:1, 20:1, 23:1, 25:1, 27:1, 29:1, 32:1, 34:1, 36:1, 38:1 or 40:1.

In some embodiments, the molar ratio of the transition metal ions and the antibody in step (A1) is 1:1 to 50:1, 1:1 to 30:1, 1:1 to 20:1, 1:1 to 15:1, 8:1 to 30:1, 12:1 to 30:1, 12:1 to 30:1, 8:1 to 16:1, 4:1 to 30:1, 4:1 to 16:1, 8:1 to 16:1, 1:1 to 10:1, 1:1 to 8:1, 1:1 to 6:1, 1:1 to 5:1, 1:1 to 4:1, 1:1 to 3:1, 1:1 to 2:1, 2:1 to 6:1, 2:1 to 4:1.

The term “transition metal ions” refers to the elements of groups 4-12, justified by their typical chemistry, i.e., a large range of complex ions in various oxidation states, colored complexes, and catalytic properties either as the element or as ions (or both). Sc and Y in Group 3 are also generally recognized as transition metals.

In some embodiments, the transition metal ions are selected from the group consisting of Zn²⁺, Cd²⁺, Hg²⁺, Ni²⁺, Co²⁺, or the combination thereof.

In some embodiments, the transition metal ions are Zn²⁺.

In some embodiments, there is no specific limitation to the salts of the transition metal ions, as long as the transition metal ions are soluble in the reaction solution so that free transition metal ions can be released in the reaction solution. In some embodiments of the present application, the salts of the transition metal ions are chloride, nitrate, sulfate, acetate, iodide, bromine, formate or tetrafluorborate.

In some embodiments, the salts of Zn²⁺ are ZnCl₂, Zn(NO₃)₂, ZnSO₄, Zn(CH₃COO)₂, ZnI₂, ZnBr₂, Zinc formate, or zinc tetrafluoroborate. In some embodiments, the salts of Zn²⁺ are ZnCl₂.

In some embodiments, there is no specific limitation to the concentration of the first reductant, as long as scaling up or down the concentration of the transition metal ions and the antibody in equal proportions. In some embodiments, the concentration of the first reductant is 0.01 mM to 0.2 mM. In some embodiments, the concentration of the first reductant is 0.02 mM to 0.15 mM. In some embodiments, the concentration of the first reductant is 0.05 mM to 0.1 mM. In some embodiments, the concentration of the first reductant is 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM or 0.20 mM.

In some embodiments, there is no specific limitation to the concentration of the transition metal ions in step (A1), as long as scaling up or down the concentration of the first reductant and the antibody in equal proportions.

In some embodiment, there is no specific limitation to the concentration of the antibody in step (A1), as long as scaling up or down the concentration of the first reductant and the transition metal ions in equal proportions.

In some embodiments, there is no specific limitation to the oxidant of step (A2), as long as the oxidant can re-oxidize the reduced thiol groups. In some embodiments, the oxidant is Dehydroascorbic acid (DHAA).

In some embodiments, in step (A2), the molar ratio of the oxidant and the antibody is 2:1 to 25:1.

In some embodiments, in step (A2), the molar ratio of the oxidant and the antibody is 4:1 to 22:1 or 3:1 to 15:1. In some embodiments, in step (A2), the molar ratio of the oxidant and the antibody is 2:1 to 15:1, 3:1 to 15:1, 6:1 to 15:1, 8:1 to 14:1, 6:1 to 10:1, 8:1 to 12:1, 6:1 to 10:1, 6:1 to 12:1, 3:1 to 8:1, 3:1 to 6:1, 5:1 to 15:1, 5:1 to 10:1, 5:1 to 8:1, 2:1 to 7:1, 4:1 to 9:1, 1:1 to 5:1, 2:1 to 4:1, or 2:1 to 6:1 in step (A2).

In some embodiments, in step (A2), the oxidation temperature is 0° C. to 37° C., and/or the oxidation time is 1 h to 48 h, optionally, in step (A2) the oxidation temperature is 0° C. to 30° C. and/or the oxidation time is 1 h to 5 h.

In some embodiments, in step (A2), the oxidation temperature is 0° C. to 37° C., 0° C. to 30° C., 0° C. to 25° C., 0° C. to 20° C., 0° C. to 15° C., 0° C. to 10° C., or 0° C. to 5° C., 5° C. to 30° C., 10° C. to 30° C., 15° C. to 30° C., 20° C. to 30° C., 0° C. to 25° C., 0° C. to 20° C., 10° C. to 25° C., 15° C. to 30° C., 5° C. to 25° C., 10° C. to 20° C. or 10° C. to 15° C. In some embodiments, in step (A2) the oxidation temperature is 0° C., 3° C., 6° C., 8° C., 10° C., 12° C., 15° C., 18° C., 20° C., 22° C., 25° C., 28° C., 30° C., 32° C., 35° C. or 37° C. In some embodiments, in step (A2) the oxidation temperature is room temperature.

In some embodiments, in step (A2), the oxidation time is 0.5 h to 15 h, 1 h to 10 h, 1 h to 5 h, 0.5 h to 5 h, 0.5 h to 3 h, 1 h to 3 h, 2 h to 5 h, 2 h to 4 h or 2 h to 3 h. In some embodiments, in step (A2), the oxidation time is 1 h, 3 h, 5 h, 7 h, 9 h, 11 h, 13 h, 15 h, 18 h, 20 h, 23 h, 25 h, 27 h, 30 h, 33 h, 35 h, 37 h, 40 h, 43 h, 45 h or 48 h.

In some embodiments, in step (A2), the oxidation reaction is in darkness.

In some embodiments, in step (A2), it is significant to improve the content of the ADC with D2, the ADC with D4 and the ADC with D1 that removing the excessive oxidant to purify the oxidized products.

In some embodiments, there is no specific limitation to the second reductant, as long as the second reductant could reduce the interchain disulfide bonds within the antibody. In some embodiments, the second reductant in step (A3) is the same as the first reductant in step (A1). In some embodiments, the second reductant in step (A3) is different with the first reductant in step (A1). In some embodiments, the second reductant in step (A3) independently is tris (2-carboxyethyl) phosphine (TCEP).

In some embodiments, in step (A3), introducing the metal chelators and the second reductant.

In some embodiments, in step (A3), the molar ratio of the metal chelators and the antibody is 2:1 to 120:1, 2:1 to 100:1, 2:1 to 80:1, 5:1 to 60:1, 10:1 to 60:1, 20:1 to 60:1, 30:1 to 60:1, 40:1 to 60:1 or 50:1 to 60:1.

In some embodiments, the molar ratio of the second reductant and the antibody in step (A3) is 10:1 to 25:1, 10:1 to 23:1, 10:1 to 20:1, 10:1 to 19:1, 10:1 to 18:1, 15:1 to 25:1, 15:1 to 20:1, 1:1 to 8:1, 1:1 to 5:1, 1:1 to 3:1, 1:1 to 2:1.

In some embodiments, in step (A3), the reduction temperature is 0° C. to 37° C., 5° C. to 25° C., 10° C. to 20° C. or 10° C. to 15° C. In some embodiments, the incubation temperature in step (A3) is room temperature.

As used herein, the term “room temperature” refers to 23° C.±2° C., 25° C.±5° C. or 20° C.±5° C.

In some embodiments, incubation time in step (A3) is 0.5 h to 12 h, 1 h to 10 h, 1 h to 8 h, 1 h to 5 h, 1 h to 3 h, 2 h to 4 h, 1 h to 4 h, or 2 h to 5 h.

In some embodiments, the reaction temperature is 0° C. to 40° C. in step (B1) and/or (B3). In some embodiments, the reaction time is 0.5 h to 10 h in step (B1) and/or (B3).

In some embodiments, the reaction temperature is 4° C. to 40° C., 10° C. to 40° C., 10° C. to 35° C., 10° C. to 30° C., 10° C. to 25° C., 15° C. to 35° C., 20° C. to 30° C. in step (B1) and/or (B3). In some embodiments, the reaction time is 0.5 h to 5 h, 0.5 h to 4 h, 0.5 h to 3 h, 0.5 h to 2 h, 0.5 h to 1 h, 1 h to 4 h, 1 h to 3 h, 1 h to 2 h, or 2 h to 4 h in step (B1) and/or (B3).

In some embodiments, the reaction is performing at room temperature in step (B1) and/or (B3).

In some embodiments, the reaction temperature is 15° C. to 25° C. in step (B1) and/or (B3), the reaction time is 1 h to 3 h in step (B1) and/or (B3).

In some embodiments, in step (B1) and/or in step (B3), the temperature of reaction with the reduced thiol groups is 4° C. to 37° C., the time of reaction with the reduced thiol groups is 0.5 h to 6 h.

In some embodiments, in step (B1) and/or in step (B3), the temperature of reaction with the reduced thiol groups is 20° C. to 30° C. or 20° C. to 25° C. In some embodiments, in step (131) and/or in step (B3), the temperature of reaction with the reduced thiol groups is room temperature. In some embodiments, in step (B1) and/or in step (B3), the temperature of reaction with the reduced thiol groups is 4° C., 6° C., 8° C., 10° C., 13° C., 17° C., 20° C., 23° C., 27° C., 30° C., 34° C. or 37° C.

In some embodiments, in step (B1) and/or in step (B3), the time of reaction with the reduced thiol groups is 0.5 h to 6 h, 0.5 h to 4 h, 0.5 h to 2 h, 1 h to 2 h or 0.5 h to 1 h. In some modifications, in step (B1) and/or in step (B3), the time of reaction with the reduced thiol groups is 0.5 h, 1 h, 2 h, 3 h, 4 h 5 h or 6 h.

In some embodiments, the temperature and time of reaction with the reduced thiol groups in step (B1) and/or in step (B3) are independent.

In some embodiments, in step (B1) and/or in step (B3), the temperature of reaction with the reactive groups is 10° C. to 37° C., the time of reaction with the reduced thiol groups is 2 h to 12 h.

In some embodiments, in step (B1) and/or in step (B3), the temperature of reaction with the reactive groups is 10° C. to 30° C., 15° C. to 30° C. or 25° C. to 30° C. In some embodiments, in step (B1) and/or in step (B3), the temperature of reaction with the reactive groups is 4° C., 6° C., 8° C., 10° C., 13° C., 17° C., 20° C., 23° C., 27° C., 30° C., 34° C., 35° C. or 37° C.

In some embodiments, in step (B1) and/or in step (B3), the time of reaction with the reactive groups is 2 h to 10 h, 4 h to 10 h, 8 h to 10 h. In some embodiments, in step (B1) and/or in step (B3), the time of reaction with the reactive groups is 2 h, 3 h, 4 h, 5 h, 6 h, 7 h, 8 h, 9 h, 10 h, 11 h, 12 h.

In some embodiments, the temperature and time of reaction with the reactive groups in step (B1) and/or in step (B3) are independent.

In some embodiments, in step (B1), according to the amount of the antibody, the first payload unit is excess.

In some embodiments, the molar ratio of the first payload units and the antibody in step (B1) is 1:1 to 50:1, 1:1 to 20:1, 1:1 to 10:1, 1:1 to 8:1, 1:1 to 6:1, 1:1 to 5:1, 2:1 to 8:1, or 2:1 to 6:1.

In some embodiments, in step (B1), the molar ratio of the first thio-bridging reagent and the antibody is 1:1 to 10:1. In some embodiment, in step (B1), the molar ratio of the first thio-bridging reagent and the antibody is 1:1, 1.05:1, 2:1, 3:1, 3.3:1, 4:1, 5:1, 6:1, 7:1, 8:1, 9:1 or 10:1.

In some embodiments, in step (B1), when the first linker-payload reacts with the reactive groups in the first thio-bridging reagent, the molar ratio of the first linker-payload and the antibody is 1:1 to 10:1. In some embodiments, in the step (B1), when the first linker-payload reacts with the reactive groups in the first thio-bridging reagent, the molar ratio of the first linker-payload and the antibody is 1:1, 1.05:1, 2:1, 3:1, 4:1, 5:1, 6:1, 7:1, 8:1, 9:1 or 10:1.

In some embodiments, in step (B1), when the first linker-payload reacts with the reduced thiol groups, the molar ratio of the first linker-payload and the antibody is 2:1 to 20:1. In some embodiments, in step (B1), when the first linker-payload reacts with the reduced thiol groups, the molar ratio of the first linker-payload and the antibody is 2:1, 3:1, 4:1, 5:1, 6:1, 20:3, 7:1, 8:1, 9:1, 10:1, 15:1 or 20:1.

In some embodiments, in step (B3), according to the amount of the antibody, the second payload unit is excess.

In some embodiments, the molar ratio of the second payload units and the antibody in step (B3) is 1:1 to 30:1, 1:1 to 20:1, 1:1 to 10:1, 1:1 to 8:1, 1:1 to 6:1, 1:1 to 5:1, 2:1 to 8:1, or 2:1 to 6:1.

In some embodiments, in step (B3), the molar ratio of the second thio-bridging reagent and the antibody is 1:1 to 10:1. In some embodiments, in step (b), the molar ratio of the second thio-bridging reagent and the antibody is 1:1, 1.5:1, 2:1, 3:1, 3.8:1, 4:1, 4.8:1 or 5:1.

In some embodiments, in step (B3), when the second linker-payload reacts with the reactive groups in the second thio-bridging reagent, the molar ratio of the second linker-payload and the antibody is 1:1 to 10:1. In some embodiments, in step (B3), when the second linker-payload reacts with the reactive groups in the second thio-bridging reagent, the molar ratio of the second linker-payload and the antibody is 1:1, 2:1, 3:1, 4:1, 5:1, 6:1, 7:1, 8:1, 9:1 or 10:1.

In some embodiments, in step (B3), when the second linker-payload reacts with the reduced thiol groups, the molar ratio of the second linker-payload and the antibody is 2:1 to 20:1. In some embodiments, in step (B3), when the second linker-payload reacts with the reduced thiol groups, the molar ratio of the second linker-payload and the antibody is 2:1, 3:1, 4:1, 5:1, 6:1, 7:1, 8:1, 9:1, 10:1, 12:1, 13:1, 14:1, 15:1, 16:1, 17:1, 18:1, 19:1 or 20:1.

In the present disclosure, the metal chelators are used to trap the excessive transition metal ions, to modify the antibody with site-specificity.

In the present disclosure, there is no specific limitation to the metal chelators, as long as the metal chelators can trap the excessive transition metal ions and do not affect the reduction of the disulfide bonds within the antibody. In some embodiments of the present application, the metal chelators are selected from a group consisting of ethylene diamine tetraacetic acid (EDTA), nitrilotriacetic acid (NTA), diethylenetriaminepentaacetic acid (DTPA), citric Acid (CA), tartaric acid (TA), gluconic acid (GA) or N-(2-hydroxyethyl) ethylenediamine-N, N′,N′-triacetic acid (HEDTA).

In some embodiments, the metal chelators are selected from a group consisting of EDTA, NTA and DTPA, or their sodium salt. In some embodiments, the metal chelators in step (B1) and (B3) is Ethylenediaminetetraacetic acid disodium salt (EDTA-2Na).

In some embodiments, the molar ratio of the metal chelators and the antibody in step (B1) is 1:1 to 100:1, 10:1 to 100:1, 20:1 to 100:1, 20:1 to 80:1, 20:1 to 70:1, 30:1 to 60:1, 40:1 to 50:1, 35:1 to 60:1, 40:1 to 55:1.

In some embodiments, the molar ratio of the metal chelators and the antibody in step (B3) is 1:1 to 100:1, 1:1 to 60:1, 1:1 to 50:1, 1:1 to 20:1, 1:1 to 10:1, 1:1 to 8:1, 1:1 to 6:1, 1:1 to 5:1, 2:1 to 8:1, 2:1 to 6:1.

In some embodiments, the second reductant in step (B2) is the same as the first reductant in step (A1). In some embodiments, the second reductant in step (B2) is different with the first reductant in step (A1). In some embodiments, the second reductant in step (B2) independently is tris (2-carboxyethyl) phosphine (TCEP).

In some embodiments, in step (B2), one of the interchain disulfide bond in the product prepared with step (A1) and (B1) is reduced completely without the transition metal ions. In some embodiments, in step (B2), three of the interchain disulfide bonds in the product prepared with step (A1), (A2) and (B1) are reduced completely without the transition metal ions. In some embodiments, one of the interchain disulfide bond or two of the interchain disulfide bonds in the product prepared with step (A1), (A2) and (B1) is(are) reduced with the transition metal ions.

In some embodiments, the molar ratio of the second reductant and the antibody in step (B2) independently is 0.05:1 to 20:1, 3:1 to 20:1, 3:1 to 10:1, 4:1 to 10:1, 5:1 to 9:1, 6:1 to 9:1, 6:1 to 8:1, 1:1 to 5:1, 1:1 to 1:4:1, 1:1 to 3:1, 1:1 to 2:1, 1.5:1 to 3:1.

In some embodiments, with step (A1), step (B1) and step (B2) without the transition metal ions, the molar ratio of the second reductant and the antibody in step (B2) independently is 1:1 to 3:1, 2:1 to 1:1, 1.5:1 to 1:1 or 1.2:1 to 1:1.

In some embodiments, with step (A1), step (A2), step (B1) and step (B2) without the transition metal ions, the molar ratio of the second reductant and the antibody in step (B2) independently is 3:1 to 20:1, 6:1 to 20:1, 4:1 to 10:1. In some embodiments, with step (A1), step (A2), step (B1) and step (B2) without the transition metal ions, the molar ratio of the second reductant and the antibody in step (B2) independently is 4:1, 5:1, 6:1, 7:1, 8:1, 9:1, 10:1, 11:1, 12:1, 13:1, 14:1, 15:1, 16:1, 17:1, 18:1, 19:1 or 20:1.

In some embodiments, in step (B2), the incubation temperature is 0° C. to 37° C., the incubation time is 0.2 h to 24 h.

In some embodiments, in step (B2), the incubation temperature is 0° C., 2° C., 4° C., 6° C., 8° C., 10° C., 12° C., 14° C., 16° C., 18° C., 20° C., 22° C., 24° C., 26° C., 28° C., 30° C., 32° C., 34° C. or 36° C.

In some embodiments, with step (A1), step (B1) and step (B2) without the transition metal ions, the incubation temperature of the second reductant in step (B2) is 5° C. to 37° C., 10° C. to 37° C., 15° C. to 37° C., 20° C. to 37° C. or 25° C. to 37° C. In some embodiments, with step (A1), step (B1) and step (B2) without the transition metal ions, the incubation temperature in step (B2) is room temperature or 37° C.

As used herein, the term “room temperature” refers to 23° C.±2° C., 25° C.±5° C. or 20° C.±5° C.

In some embodiments, with step (A1), step (A2), step (B1) and step (B2), the incubation temperature of the second reductant in step (B2) is 0° C. to 30° C., 0° C. to 25° C., 0° C. to 20° C., 0° C. to 15° C. or 0° C. to 10° C.

In some embodiments, with step (A1), step (A2), step (B1) and step (B2), the incubation temperature in step (B2) is 4° C., 6° C., 8° C., 10° C., 12° C. or 14° C.

In some embodiments, incubation time in step (B2) independently is 0.5 h to 24 h, 1 h to 10 h, 1 h to 8 h, 1 h to 5 h, 1 h to 3 h, 2 h to 4 h, 1 h to 4 h, or 2 h to 5 h. In some embodiments, in step (B2), the incubation temperature is 1 h, 2 h, 3 h, 4 h, 5 h, 6 h, 7 h, 8 h, 9 h, 10 h, 12, 14 h, 16 h, 18 h, 20 h, 22 h or 24 h.

In some embodiments, with step (A1), step (B1) and step (B2) without the transition metal ions, the incubation time of the second reductant in step (B2) is 0.5 h to 18 h, 0.5 h to 12 h, 1 h to 10 h, 1 h to 8 h, 1 h to 5 h, 1 h to 3 h, 2 h to 4 h, 1 h to 4 h, or 2 h to 5 h. In some embodiments, with step (A1), step (B1) and step (B2) without the transition metal ions, the incubation time in step (B2) is 1 h, 2 h, 3 h, 4 h, 5 h, 6 h, 7 h, 8 h, 9 h or 10 h.

In some embodiments, with step (A1), step (A2), step (B1) and step (B2) without the transition metal ions, the incubation time in step (B2) is 3 h, 4 h, 5 h, 6 h, 7 h, 8 h, 9 h, 10 h, 12 h, 14 h, 16 h, 18 h, 20 h, 22 h or 24 h.

In some embodiments, introducing the transition metal ions, two of the interchain disulfide bonds are selectively reduced. In some embodiments, with step (A1), step (A2), step (B1) and step (B2), in step (B2), the molar ratio of the second reductant and the transition metal ions is 1:0.05 to 1:40, and/or the molar ratio of the second reductant and the antibody is 2.5:1 to 20:1, and/or the incubation time is 1 h to 24 h. In some embodiments, with step (A1), step (A2), step (B1) and step (B2), in step (B2), the molar ratio of the second reductant and the transition metal ions is 1:0.05, 1:0.08, 1:0.1, 1:0.2, 1:0.3, 1:0.4, 1:0.5, 1:0.6, 1:0.7, 1:0.8, 1:0.9, 1:1, 1:2, 1:4, 1:6, 1:8, 1:10, 1:12, 1:14, 1:16, 1:18 or 1:20. In some embodiments, with step (A1), step (A2), step (B1) and step (B2), in step (B2), the molar ratio of the second reductant and the antibody is 2.5:1, 3:1, 5:1, 7:1, 9:1, 11:1, 13:1, 15:1, 17:1, 19:1 or 20:1. In some embodiments, with step (A1), step (A2), step (B1) and step (B2), in step (B2), the incubation time is 1 h, 2 h, 4 h, 6 h, 8 h, 10 h, 12 h, 14 h, 16 h, 18 h, 20 h, 22 or 24 h. In some embodiments, with step (A1), step (A2), step (B1) and step (B2), in step (B2), the molar ratio of the second reductant and the transition metal ions is 1:0.05 to 1:40, and/or the molar ratio of the second reductant and the antibody is 3:1 to 15:1, and the incubation time is 1 h to 12 h. In some embodiments, with step (A1), step (A2), step (B1) and step (B2), in step (B2), the molar ratio of the second reductant and the transition metal ions is 1:0.05 to 1:40, and/or the molar ratio of the second reductant and the antibody is 2.5:1 to 15:1, and the incubation time is 12 to 24 h.

In some embodiments, introducing the transition metal ions, one of the interchain disulfide bonds are selectively reduced. In some embodiments, with step (A1), step (A2), step (B1) and step (B2), in step (B2), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h. In some embodiments, with step (A1), step (A2), step (B1) and step (B2), in step (B2), the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, with step (A1), step (A2), step (B1) and step (B2), in step (B2), the molar ratio of the second reductant and the antibody is 0.8:1, 1:1, 1.2:1, 1.4:1, 1.6:1, 1.8:1, 2:1, 2.2:1, 2.4:1, 2.5:1. In some embodiments, with step (A1), step (A2), step (B1) and step (B2), in step (B2), the incubation time is 0.5 h, 1 h, 2 h, 4 h, 6 h, 8 h, 10 h, 12 h, 14 h, 16 h, 18 h, 20 h, 22 or 24 h. In some embodiments, with step (A1), step (A2), step (B1) and step (B2), in step (B2), the molar ratio of the second reductant and the transition metal ions is 1:0.4 to 1:100, and/or the molar ratio of the second reductant and the antibody is 0.8:1 to 2:1, and the incubation time is 0.5 h to 24 h. In some embodiments, with step (A1), step (A2), step (B1) and step (B2), in step (B2), the molar ratio of the second reductant and the transition metal ions is 1:0.54 to 1:100, and/or the molar ratio of the second reductant and the antibody is 2:1 to 2.5:1, and the incubation time is 1 h to 9 h.

Without to bound by any theory, the reductant selectively reduces disulfide bonds in buffer system. The buffer system of step (A1), (A3) and (B2) independently is MES buffer, Bis-Tris buffer, PIPES buffer, MOPS buffer, BES buffer, HEPES buffer, DIPSO buffer, MOBS buffer, MOPSO buffer, TES buffer, ACES buffer, TAPSO buffer, PBS, Acetate buffer, ADA buffer BTP buffer, HEPPSO buffer, POPSO buffer, EPPS buffer or Tris buffer.

As used herein, the term “MES buffer” refers to 2-(N-morpholino) ethane sulfonic acid buffer.

As used herein, the term “Bis-Tris buffer” refers to Bis(2-hydroxyethyl) amino-tris(hydroxymethyl)methane buffer.

As used herein, the term “PIPES buffer” refers to piperazine-1,4-bisethanesulfonic acid buffer.

As used herein, the term “MOPS buffer” refers to 3-morpholinopropanesulfonic Acid buffer.

As used herein, the term “BES buffer” refers to N, N-Bis (2-hydroxyethyl)-2-aminoethanesulphonic acid buffer.

As used herein, the term “HEPES buffer” refers to 4-hydroxyethyl piperazine ethane sulfonic acid buffer.

As used herein, the term “DIPSO buffer” refers to 3-[bis(2-hydroxyethyl) amino]-2-hydroxypropanesulphonic acid buffer.

As used herein, the term “MOBS buffer” refers to 3-morpholinopropanesulfonic Acid buffer.

As used herein, the term “MOPSO buffer” refers to 3-(N-morpholino)-2-hydroxy-1-propanesulfonic acid buffer.

As used herein, the term “TES buffer” refers to 2-[tris(hydroxymethyl)methylamino]-1-ethanesulfonic acid buffer.

As used herein, the term “ACES buffer” refers to N-(carbamoylmethyl)taurine buffer.

As used herein, the term “TAPSO buffer” refers to 3-[N-tris-(hydroxymethyl) methylamino]-2-hydroxypropanesulphonic acid buffer.

As used herein, the term “PBS” refers to phosphate buffer saline.

As used herein, the term “ADA buffer” refers to N-(Carbamoylmethyl) iminodiacetic acid buffer.

As used herein, the term “PB buffer” refers to refers to phosphate buffer.

As used herein, the term “BTP buffer” refers to Bis-tris propane buffer.

As used herein, the term “Heppso buffer” refers to N-(Hydroxyethyl) piperazine-N′-2-hydroxypropanesulfonicacid buffer.

As used herein, the term “POPSO buffer” refers to piperazine-N, N′-bis(2-hydroxy-propane sulfonic) acid buffer.

As used herein, the term “EPPS buffer” refers to 4-(2-Hydroxyethyl)-1-piperazinepropanesulfonic acid buffer.

As used herein, the term “Tris buffer” refers to tris(hydroxymethyl)aminomethane buffer.

In some embodiments, the buffer system of step (A1), (A3) and (B2) independently is MES buffer, Bis-Tris buffer, MOPS buffer, BES buffer, HEPES buffer, DIPSO buffer, MOBS buffer, MOPSO buffer, TES buffer, ACES buffer or TAPSO buffer.

In some embodiments, the buffer system of step (A1), (A3) and (B2) independently is Bis-Tris buffer, MOPS buffer, BES buffer. HEPES buffer, DIPSO buffer, MOBS buffer, MOPSO buffer, TES buffer, ACES buffer or TAPSO buffer.

In some embodiments, the buffer system is BES buffer.

In some embodiments, the pH value of the buffer system is 5.8 to 8.0. In some embodiments, the pH value of the buffer system is 6.0 to 7.4. In some embodiments, the pH value of the buffer system is 6.7 to 7.4. In some embodiments, the pH value of the buffer system is 6.0, 6.2, 6.5, 6.8, 7.0, 7.2 or 7.4.

In some embodiments, the buffer system of step (A1) and (A3) is same. In some embodiments, the buffer system of step (A1) and (A3) is different. In some embodiments, the buffer system of step (A1) and (B2) is same. In some embodiments, the buffer system of step (A1) and (B2) is different.

The concentration of the buffer system of step (A1), (A3) and (B2) in the method independently is ranging from 10 mM to 100 mM (mmol/L), optionally, the concertation of the buffer system of step (A1), (A3) or (B2) in the method is 20 mM to 100 mM, 20 mM to 80 mM, 20 mM to 60 mM, 20 mM to 40 mM, 40 mM to 80 mM, 40 mM to 60 mM, 30 mM to 80 mM, 30 mM to 60 mM, 50 mM to 80 mM, or 30 mM to 70 mM.

In some embodiments, the concentration of buffer system of step (A1) and (A3) is same. In some embodiments, the concentration of buffer system of step (A1) and (A3) is different. In some embodiments, the concentration of buffer system of step (A1) and (B2) is same. In some embodiments, the concentration of buffer system of step (A1) and (B2) is different.

In some embodiments, the method also comprises the following step: introducing a compound which contains at least one thiol group to consume the excessive first linker-payload and the excessive second linker-payload. In some embodiments, the compound is cysteine.

In some embodiments, the method also comprises the following step: purifying and recovering the product from (B1) and/or (B3).

In some embodiments, the resultant antibody-drug conjugates are recovered by any suitable purification method, such as using a de-salting column, size exclusion chromatography, ultrafiltration, dialysis, ultrafiltration (UF)-diafiltration (DF), and the like. If needed, further ADC enrichment (e.g., D2) may be applied in some case using hydrophobic interaction chromatography (HIC).

In some embodiments, in step (B1) and/or (B3), the resultant ADC is purified by a desalting column, size exclusion chromatography, ultrafiltration, dialysis and/or the like. In some embodiments of the present application, in step (B1) and/or (B3), the resultant ADC is purified by a desalting column.

In some embodiments, a linker of the first linker-payload and the second liner payload is selected from any one of which the one terminal can be connected to the reduced thiol group of the antibody or the reactive groups of the thio-bridging reagent, and the other terminal can be connected to a payload of the payload.

As used herein, the term “linker” refers to a substituted molecule which contains at least two substituted groups, one of which can covalently bond a drug molecule and the other of which can covalently couple to an antibody or the reactive groups of the thio-bridging reagent.

In some embodiments, when the first linker-payload and/or the second linker-payload react(s) with the reduced thiol groups, the linker of the first linker-payload and the second linker-payload independently includes a cleavable linker or a noncleavable linker. Cleavable linkers can be chemically labile and enzyme-labile linkers. Due to the high plasma stability and good intracellular cleaving selectivity and efficiency, enzyme-labile linkers are broadly selected as cleavable linker candidates in ADCs. In some embodiments, enzyme-labile linkers may include a peptide unit (-AAs-), -Maleimidocaproyl-(-MC-), -p-aminobenzyl alcohol-(-PAB-), or -MC-peptide unit-PAB-. In some embodiments, the peptide unit is dipeptides, tripeptides, tetrapeptides or pentapeptides.

In some embodiments, without the limitation, the dipeptides can be valine-alanine (VA), valine-citrulline (VC), alanine-asparagine (AD), alanine-phenylalanine (AF), phenylalanine-lysine (FK), alanine-lysine (AK), alanine-valine (AV), valine-lysine (VK), lysine-lysine (KK), phenylalanine-citrulline (FC), leucine-citrulline (LC), isoleucine-citrulline (IC), tryptophan-citrulline (WC) or phenylalanine-alanine (FA). In some embodiments, the dipeptides can be valline-citruline-(-Val-Cit-), -valline-lysine-(-Val-Lys-), -valline-arginine-(-Val-Arg-), -phenylalanine-citruline-(-Phe-Cit-), -phenylalanine-lysine-(-Phe-Lys-), and -phenylalanine-arginine-(-Phe-Arg-). Typical enzyme-labile linkers include -Val-Cit- and -Phe-Lys-, which can be recognized by cathepsin B.

In some embodiments, without the limitation, the tripeptides can be alanine-alanine-asparagine (AAD), glycine-valine-citrulline (GVC), glycine-glycine-glycine (GGG), phenylalanine-phenylalanine-lysine (FFK), glutamic acid-valine-citrulline (EVC), or glycine-phenylalanine-lysine (GFK).

In some embodiments, without the limitation, the tetrapeptides can be glycine-glycine-phenylalanine-glycine (GGFG).

In some embodiments, without the limitation, when the first linker-payload and/or the second linker-payload react(s) with the reduced thiol groups, the linker of the first linker-payload and the second linker-payload can be MC-VA-PAB, MC-VC-PAB, MC-AD-PAB, MC-AF-PAB, MC-FK-PAB, MC-AK-PAB, MC-AV-PAB, MC-VK-PAB, MC-KK-PAB, MC-FC-PAB, MC-LC-PAB, MC-IC-PAB, MC-WC-PAB or MC-FA-PAB independently. In some embodiments, without the limitation, when the first linker-payload and/or the second linker-payload react(s) with the reduced thiol groups, the linker of the first linker-payload and the second linker-payload can be MC-AAD-PAB, MC-GVC-PAB, MC-GGG-PAB, MC-FFK-PAB, MC-EVC-PAB, or MC-GFK-PAB independently. In some embodiments, without the limitation, when the first linker-payload and/or the second linker-payload react(s) with the reduced thiol groups, the linker of the first linker-payload and the second linker-payload can be MC-GGFG.

In some embodiments, the linker comprises a maleimide bearing a drug, an organic chloride bearing a drug, an organic bromide bearing a drug, an organic iodide bearing a drug and/or vinylpyrimidine bearing a drug.

In some embodiments, the linker includes a maleimide bearing a drug, an organic chloride bearing a drug, an organic bromide bearing a drug, an organic iodide bearing a drug and/or vinylpyrimidine bearing a drug.

In some embodiments, when the first linker-payload and/or the second linker-payload react(s) with the reactive groups in the thio-bridging reagent, the linker of the first linker-payload and/or the second linker-payload further include(s) azido and/or dibenzocyclooctyne (DBCO). In some embodiments, when the linker of the first linker-payload and/or the second linker-payload contains azido, the reactive groups of the thio-bridging group contain DBCO. In some embodiments, when the linker of the first linker-payload and/or the second linker-payload contains DBCO, the reactive groups of the thio-bridging group contain azido.

In some embodiments, when the linker of the first linker-payload and/or the second linker-payload react(s) with the reactive groups in the thio-bridging reagent, the linker of the first linker-payload and the second linker-payload is independently selected from any one of the groups consisting of

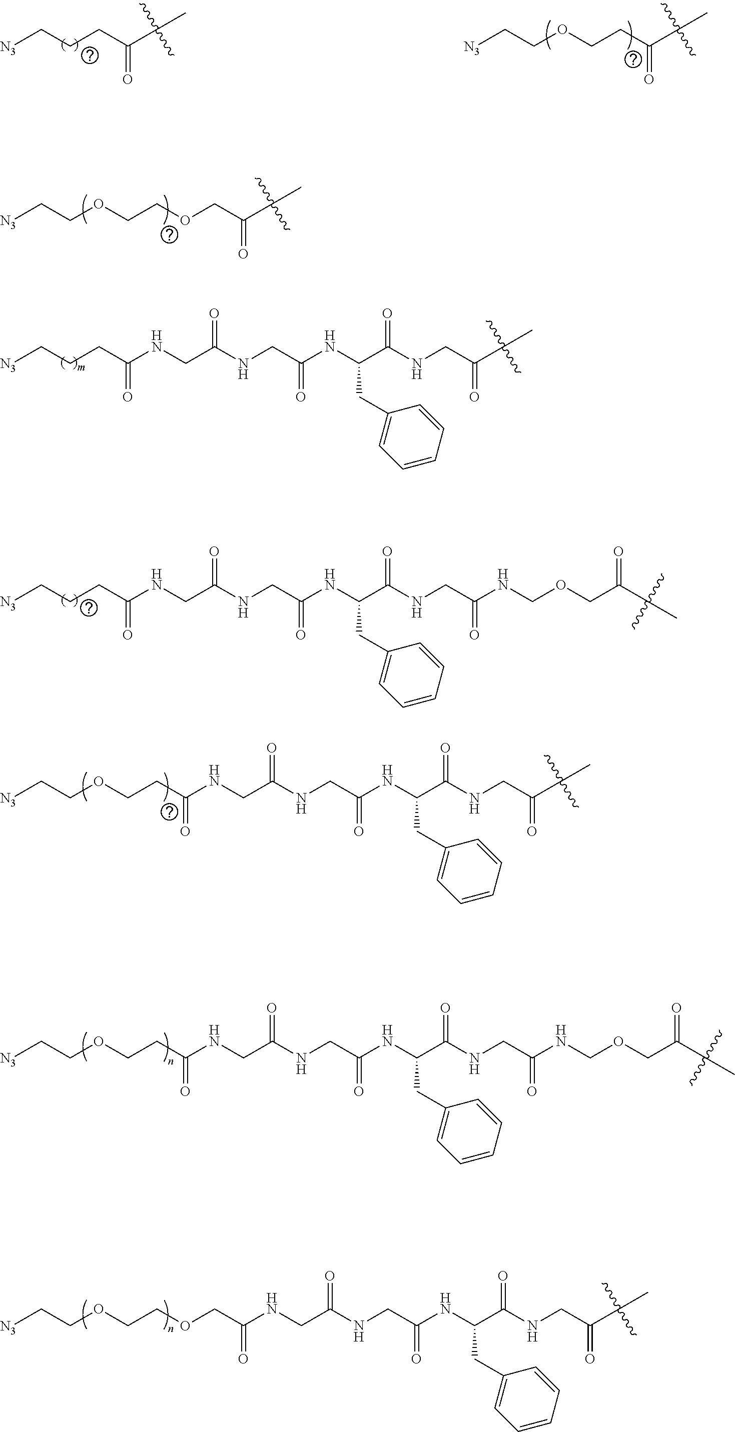

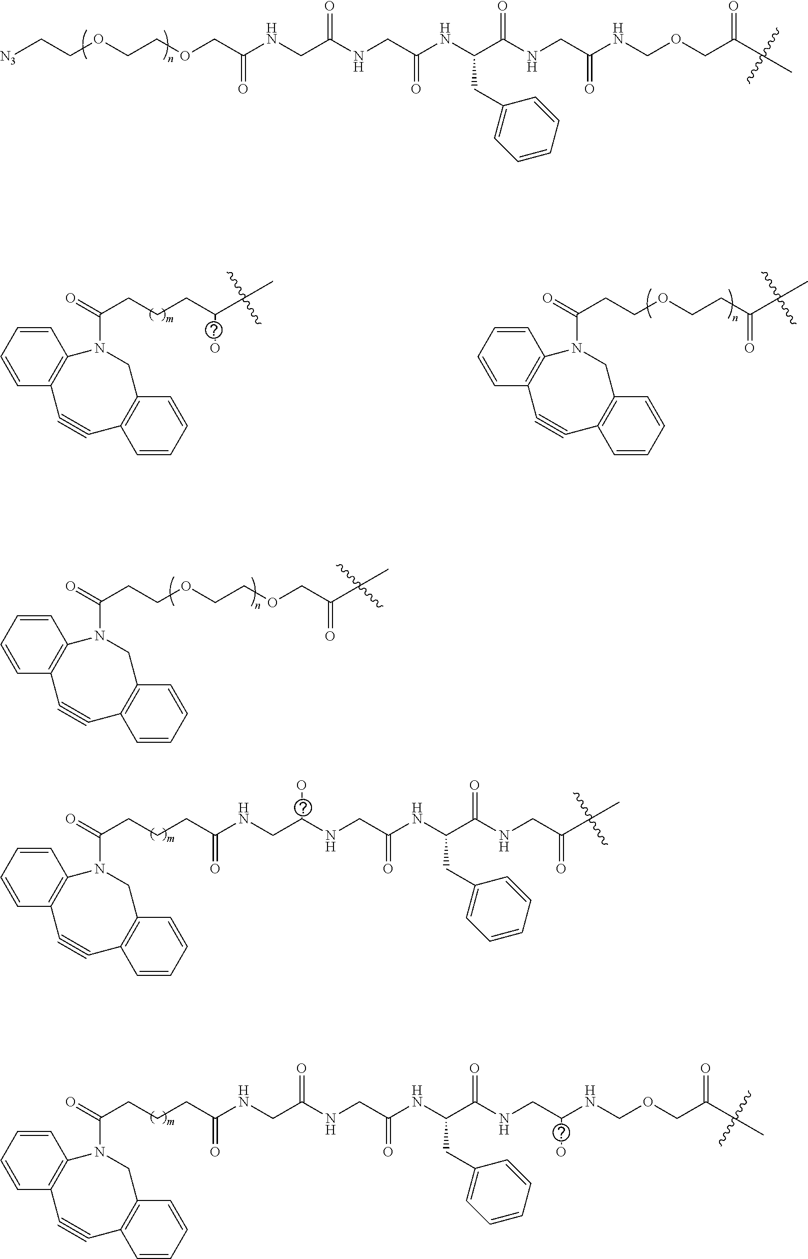

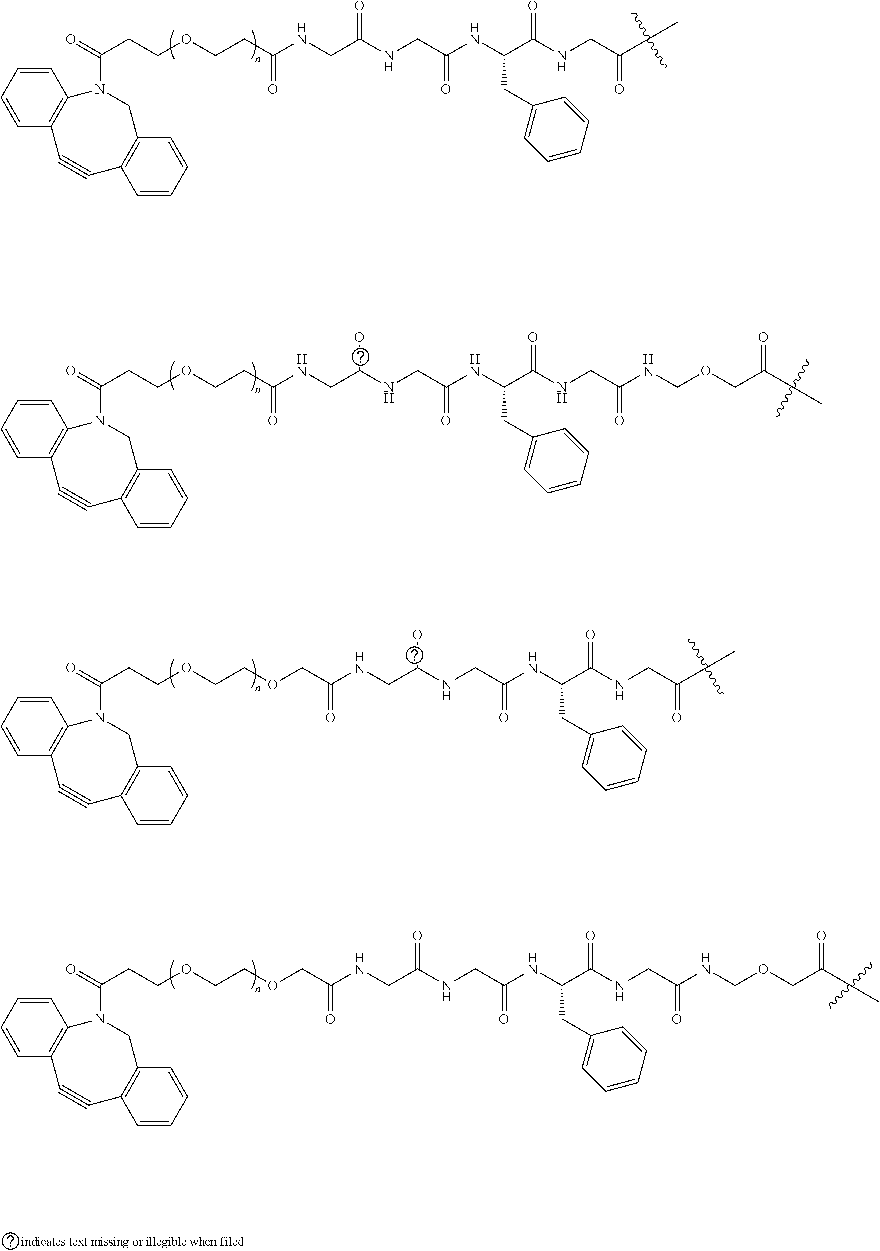

wherein, n of the linker is integer of 0-20, 0-18, 0-15, 0-13, 0-10, 0-7, 0-5 or 0-3, m is integer of 0-20, 0-18, 0-15, 0-13, 0-10, 0-7, 0-5 or 0-3, optionally, n is 0, 1, 2, 3, 4, 5, 6, 7, 8, 9 or 10, m is 0, 1, 2, 3, 4, 5, 6, 7, 8, 9 or 10.

As used herein, the term “payload” refers to any cytotoxic molecule at least one substituted group or a partial structure allowing connection to a linker structure. The payload may kill cancer cells and/or inhibit growth, proliferation, or metastasis of cancer cells, thereby reducing, alleviating, or eliminating one or more symptoms of a disease or disorder.

In some embodiments, the payload is a cytotoxic drug, a cytokine, a nucleic acid, a radionuclide, a kinase or derivatives thereof. In some embodiments, the payload includes but not limited to topoisomerases inhibitor and tubulin inhibitors.

Exemplary payloads are monomethyl auristatin E (MMAE), monomethyl auristatin D (MMAD), monomethyl auristatin EF(MMAF), calicheamicins (CLM), mertansine (DM1), maytansinoids, duocarmycins, anthracyclines, pyrrolobenzodiazepine dimers, amatoxin, quinolinealkaloid, Dxd, doxorubicin hydrochloride, methotrexate, erlotinib, bortezomib, fulvestrant, sunitib imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, vincristine, vinblastine, vinorelbine, vindesine, etoposide, teniposide, etoposide phosphate, epipodophyllotoxins, actinomycin, daunorubicin, valrubicin, idarubicin, edrecolomab, epirubicin bleomycin, plicamycin or mitomycin, and salts thereof.

In some embodiments, the payload is deruxtecan (DXd), cyanine 3 (Cy3), MMAE, MMAD or MMAF. In some embodiments of the present application, the payload is MMAE, DXd or Cy3.

The linker-payload is a chemical moiety, which is synthesized by connecting a linker to a payload. Depending on the desired payload and selected linker, those skilled in the art can select suitable method for coupling them together. For example, some conventional coupling methods, such as amine coupling methods, may be used to form the desired linker-payload which still contains reactive groups for conjugating to the antibodies through covalent linkage. A drug-maleimide complex (i.e., maleimide linking drug) is taken as an example of the payload bearing reactive group in the present disclosure. Most common reactive group capable of bonding to thiol group in ADC preparation is maleimide. Additionally, organic chloride, bromides, iodides also are frequently used.

The linker-payload could be any physical active compound, or any compound used to diagnose, prevent or treat a disease, such as MC-GGFG-DXd, MC-VC-PAB-MMAE, MC-VC-PAB-MMAD, and MC-VC-PAB-MMAF.

In some embodiments, the first linker-payload and the second linker-payload are same. In some embodiments, the first linker-payload and the second linker-payload are different.

The first thio-bridging reagent and the second thio-bridging reagent independently contain at least two substituted groups allowing a re-bridging of the thiol groups.

In some embodiments, without the limitation, the first thio-bridging reagent and the second thio-bridging reagent are independently selected from the group consisting of

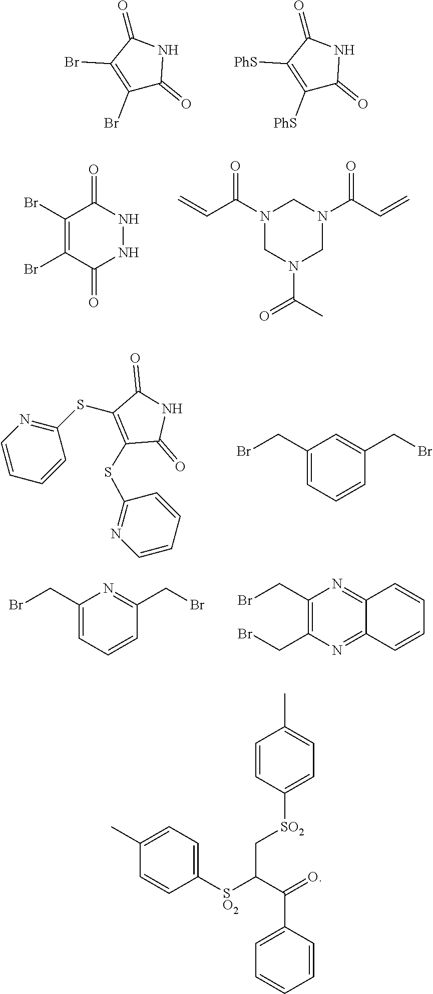

In some embodiments, the reactive groups contain azido and/or dibenzocyclooctyne (DBCO).

In some embodiments, the thio-bridging reagent and the reactive groups are connected by alkyl group or polyethylene glycol (PEG).

In some embodiments, without the limitation, the first thio-bridging reagent bearing reactive groups and the second thio-bridging reagent bearing reactive groups are independently selected from the groups consisting of

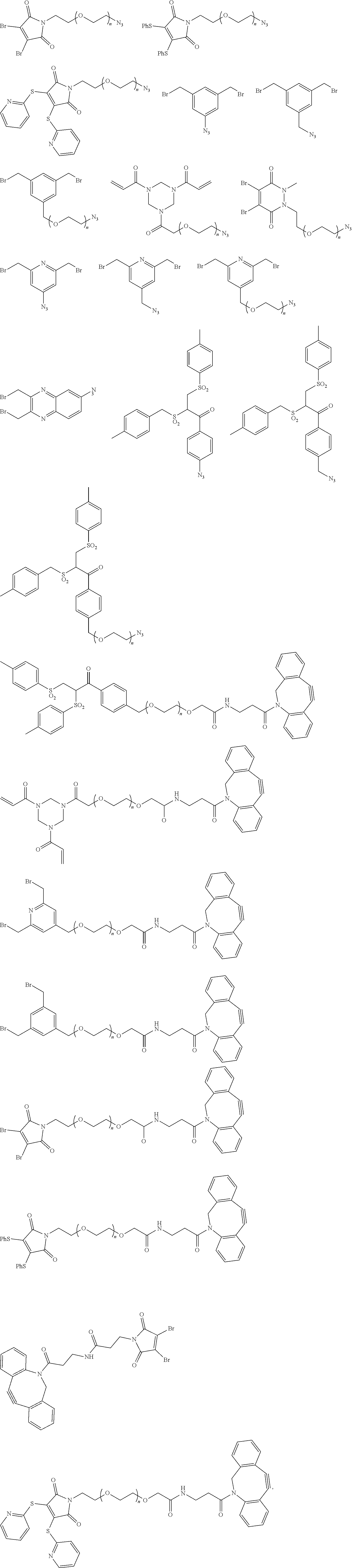

wherein, n of the first thio-bridging reagent bearing reactive groups and the second thio-bridging reagent bearing reactive groups is integer of 0-20, 0-18, 0-15, 0-13, 0-10, 0-7, 0-5 or 0-3.

In some embodiments, the first thio-bridging reagent bearing reactive groups could be different from the second thio-bridging reagent bearing reactive groups. In some embodiments, the first thio-bridging reagent bearing reactive groups could be the same as the second thio-bridging reagent bearing reactive groups.

In some embodiments, the first thio-bridging reagent bearing reactive groups and the second thio-bridging reagent bearing reactive groups are dibromomaleimide-PEG4-N3 having the following formula

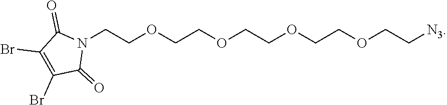 [SMILES: [N-]=[N+]=NCCOCCOCCOCCOCCN1C(=O)C(Br)=C(Br)C1=O]

On appropriate conditions, the reactive groups could react with the linker-payloads, and the linker-payload is connected to thio-bridging reagent by covalence. With the change of reactive groups or linker-payloads, the reaction products maybe change. In the disclosure, the products of different reactive groups and linker-payloads are collectively referred to as thio-bridging reagent bearing linker-payload.

In some embodiments, the first thio-bridging reagent bearing the first linker-payload and the second thio-bridging reagent bearing the second linker-payload have the following formula:

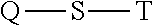

Wherein, Q is selected from the groups consisting of

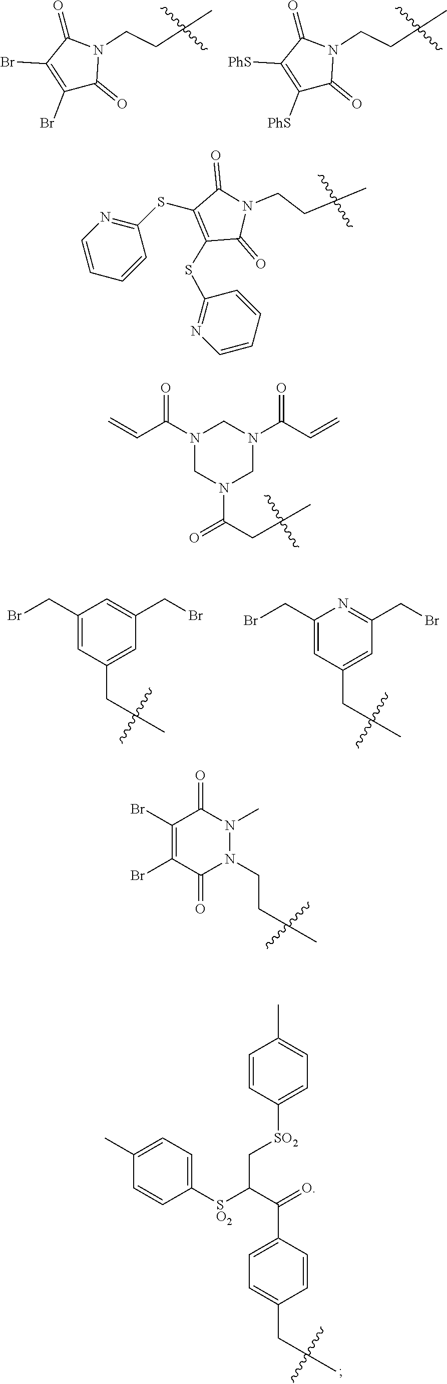

S is selected from a cleavable linker or a non-cleavable linker, without the limitation, S is selected from the groups consisting of

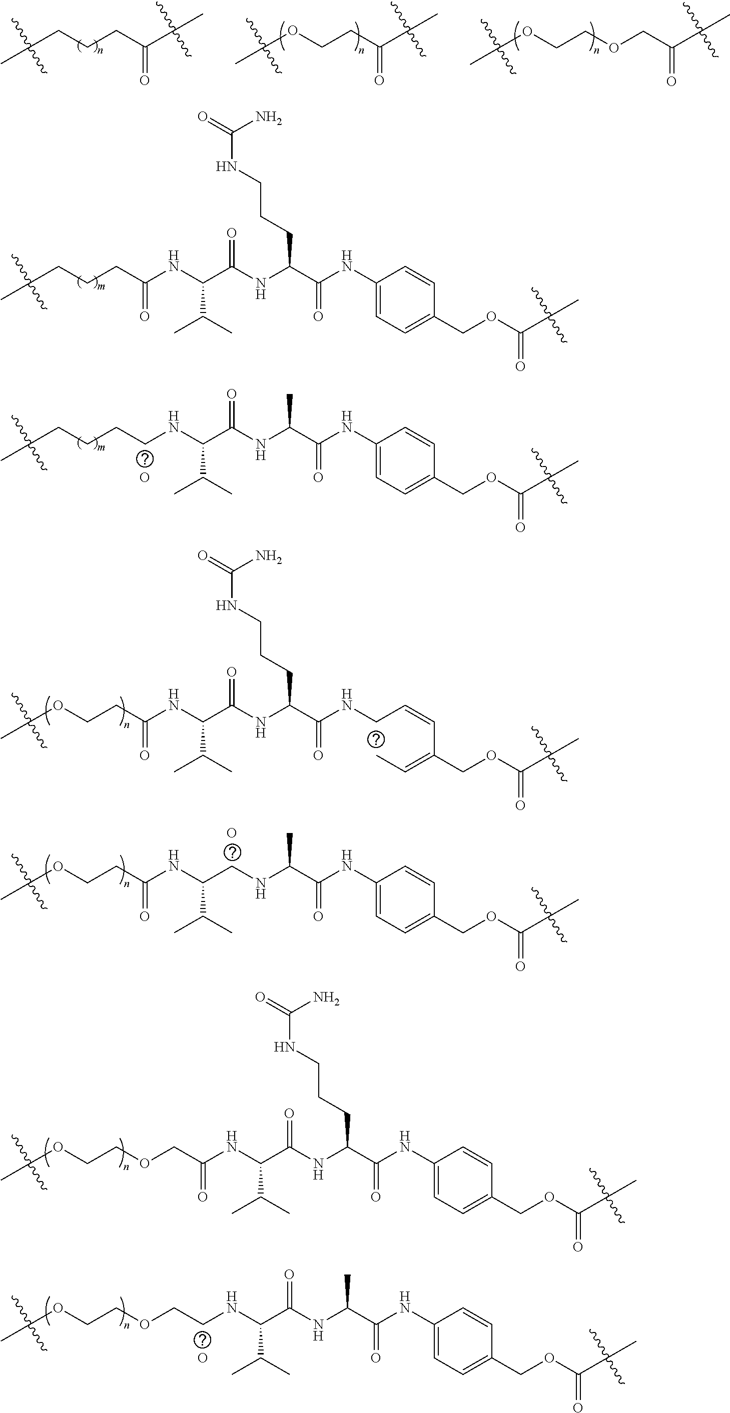

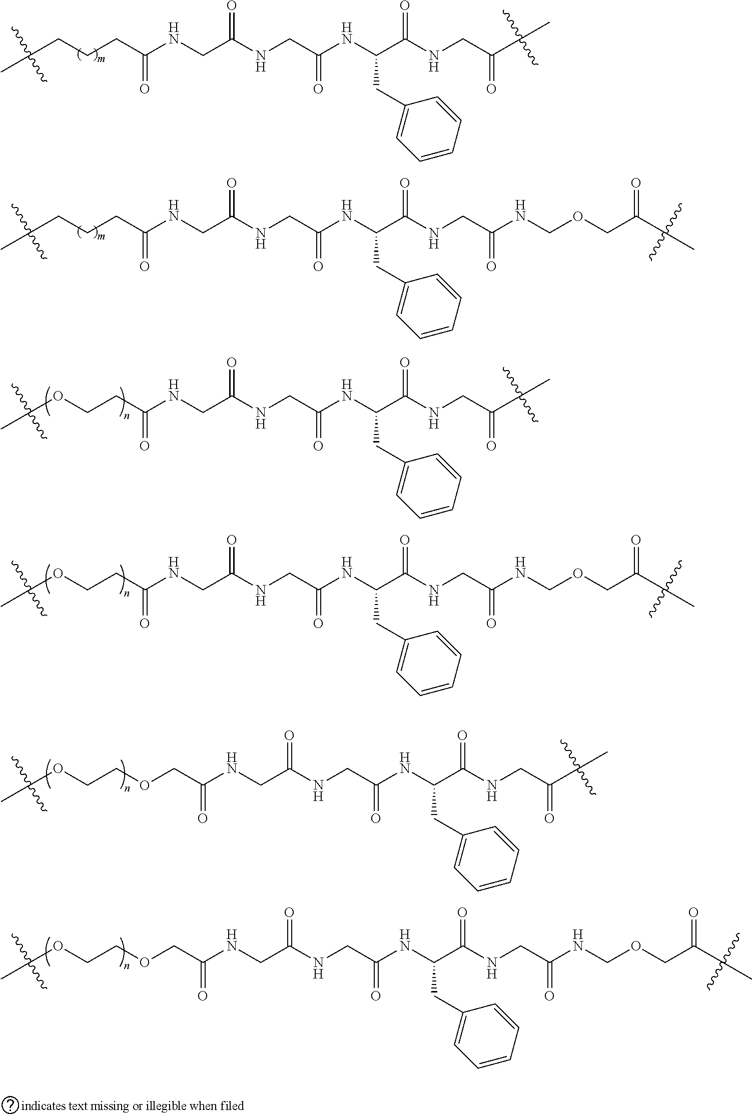

wherein, n is 0-20, m is 0-20, optionally, n is 0, 1, 2, 3, 4, 5, 6, 7, 8, 9 or 10, m is 0, 1, 2, 3, 4, 5, 6, 7, 8, 9 or 10.

T is payload.

In some embodiments, the first thio-bridging reagent bearing the first linker-payload and the second thio-bridging reagent bearing the second linker-payload, without the limitation, are selected from the groups consisting of

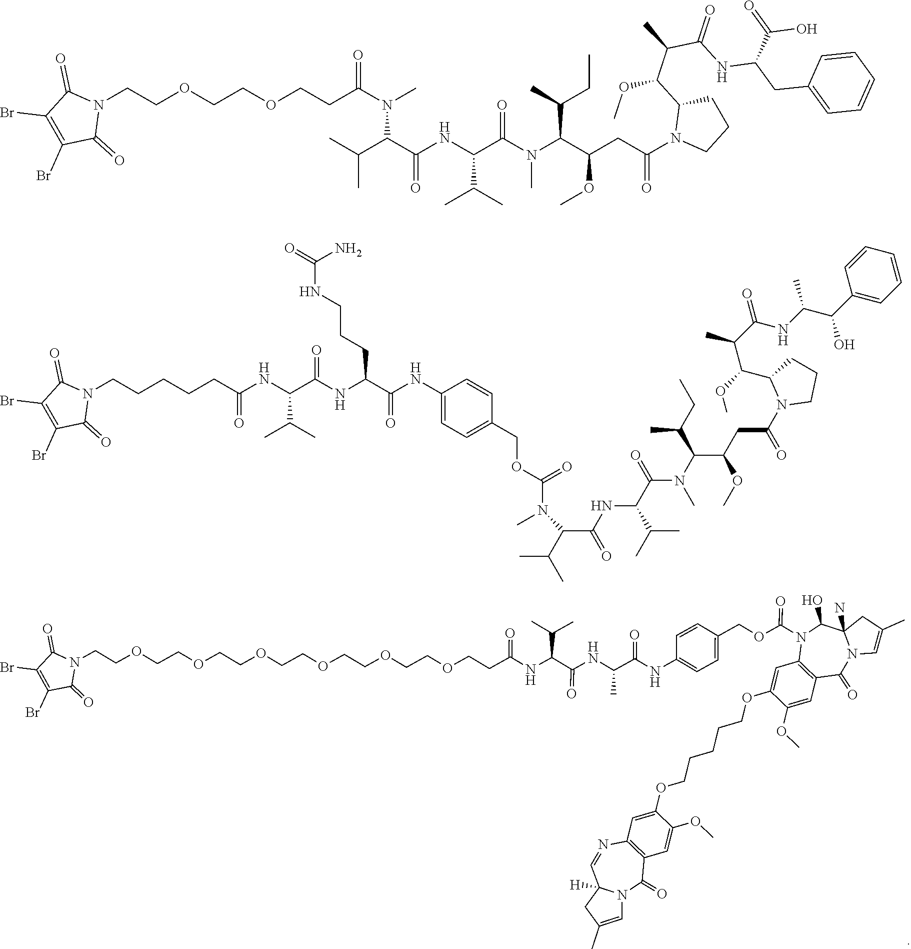

Thus, the first payload units include the first linker-payload, the first thio-bridging reagent bearing reactive groups, the first thio-bridging reagent bearing first linker-payload.

The second payload units include the second linker-payload, the second thio-bridging reagent bearing reactive groups, the second thio-bridging reagent bearing the second linker-payload.

In some embodiments, the first payload units are the first linker-payloads, the first thio-bridging reagent bearing reactive groups or the first thio-bridging reagent bearing first linker-payload.

In some embodiments, the second payload units are the second linker-payloads, the second thio-bridging reagent bearing reactive groups, or the second thio-bridging reagent bearing the second linker-payload.

In some embodiments, the payload of the first thio-bridging reagent bearing the first linker-payload and that of the second thio-bridging reagent bearing the second linker-payload are different. In some embodiments, the linker of the first thio-bridging reagent bearing the first linker-payload and that of the second thio-bridging reagent bearing the second linker-payload could be different. In some embodiments, the linker of the first thio-bridging reagent bearing the first linker-payload and that of the second thio-bridging reagent bearing the second linker-payload could be the same. In some embodiments, the thio-bridging reagent of the first thio-bridging reagent bearing the first linker-payload and that of the second thio-bridging reagent bearing the second linker-payload could be different. In some embodiments, the thio-bridging reagent of the first thio-bridging reagent bearing the first linker-payload and that of the second thio-bridging reagent bearing the second linker-payload could be the same.

As used herein, the term “end capping reagent” refers to a compound which does not bear a drug and contains at least one substituted group which can covalently couple to an antibody.

In some embodiments, the end capping reagent is the cleavable linker or the noncleavable linker. In some embodiments, the end capping reagent is (2-Aminoethyl) maleimide.

In some embodiments, the antibody is a monoclonal antibody, a polyclonal antibody, a mono-specific antibody or a multi-specific antibody.

As used herein, the term “antibody” refers to any immunoglobulin that binds to a specific antigen.

A native intact antibody includes two heavy chains and two light chains. Each heavy chain consists of a variable region and a first, second, and third constant region, while each light chain consists of a variable region and a constant region. The heavy chain from any vertebrate species can be assigned to one of five different classes (or isotypes): IgA, IgD, IgE, IgG, and IgM.

As used herein, the term “hinge region” refers to an antibody includes the portion of a heavy chains molecule that joins the CH1 domain to the CH₂ domain. This hinge region includes approximately 25 amino acid residues and is flexible, thus allowing the two N-terminus antigen binding regions to move independently.

As used herein, the term “Fab fragments” refers to the region of the antibody structure that can bind to antigen. It consists of a complete light chain (variable and constant regions) and part of the heavy chain structure (variable and a constant region fragment), the light and heavy chains are connected by a disulfide bond. Fab fragments can be obtained by protease digestion of full-length antibodies. Under the action of papain, human immunoglobulin G can be degraded into two Fab fragments and one Fc fragment; under the action of pepsin, IgG can be degraded into an F(ab′)2 fragment and a pFc′ fragment. The F(ab′)2 fragment can be further reduced to form two Fab′ fragments.

As used herein, the term “Fc region” refers to a monomeric, dimeric or heterodimeric protein having at least an immunoglobulin CH2 and CH3 domain. The CH2 and CH3 domains can form at least a part of the dimeric region of the protein/molecule (e.g., antibody).

In some embodiments, the antibody is a human antibody, a humanized antibody, a chimeric antibody, or an antigen-binding moiety thereof.

As used herein, the term “human antibody” refers to one which possesses an amino acid sequence which corresponds to that of an antibody produced by a human or a human cell or derived from anon-human source that utilizes human antibody repertoires or other human antibody-encoding sequences. This definition of a human antibody specifically excludes a humanized antibody comprising non-human antigen-binding residues.

As used herein, the term “humanized antibody” refers to a chimeric antibody comprising amino acid residues from non-human heavy chain variable regions (HVRs) and amino acid residues from human FRs. In certain embodiments, a humanized antibody will include substantially all or at least one, and typically two, variable domains, in which all or substantially all of the HVRs (e.g., CDRs) correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

As used herein, the term “chimeric antibody” refers to an antibody in which a portion of the heavy and/or light chain is derived from a particular source or species, while the remainder of the heavy and/or light chain is derived from a different source or species.

There is no specific limitation to the antibody. According to the antigens associated with the disease, those skilled in the art can select suitable antibody useful in the bio-conjugation process of the present application.

In some embodiments of the present application, the antibody means an immunoglobulin and is a molecule containing an antigen-binding site immunospecifically binding to an antigen. In some embodiments of the present application, the class of the antibody is IgG, IgE, IgM, IgD, IgA, or IgY. In some embodiments of the present application, the class of the antibody is IgG.

In some embodiments of the present application, the class of the antibody is IgG1, IgG2, IgG3 or IgG4. In some embodiments, the antibody is IgG1 or IgG4.

In some embodiments of the present application, the antibody is wild type. As use herein, the term “wild type” refers to naturally occurring and without mutation.

In some embodiments of the present application, the antibody includes at least one mutation in the Fc region. In some embodiments, the at least one mutation modulates effector function, or attenuates or eliminates Fc-g receptor binding.

In some embodiments, the one or more mutations are to stabilize the antibody and/or to increase half-life. In some instances, the one or more mutations are to modulate Fe receptor interactions, to reduce or eliminate Fe effector functions such as FcyR, antibody-dependent cell-mediated cytotoxicity (ADCC) or complement-dependent cytotoxicity (CDC). In additional instances, the one or more mutations are to modulate glycosylation.

In some embodiments, the one or more mutations are located in the Fc region. In some instances, the Fc region includes a mutation at residue position L234, L235, or a combination thereof. In some instances, the mutations include L234 and L235. In some instances, the mutations include L234A and L235A. In some cases, the residue positions are in reference to IgG1.

In some embodiments, the Fc region includes a mutation at residue position L234, L235, D265, N21, K46, L52, or P53, or a combination thereof. In some instances, the mutations include L234 and L235 in combination with a mutation at residue position K46, L52, or P53. In some cases, the residue positions are in reference to IgG1.

In some embodiments, the Fc region includes mutations at L234, L235, and K46. In some cases, the Fc region includes mutations at L234, L235, and L52. In some cases, the Fe region includes mutations at L234, L235, and P53. In some cases, the Fe region includes mutations at D265 and N21. In some cases, the residue position is in reference to IgG1.

In some instances, the Fc region includes L234A, L235A, D265A, N21G, K46G, L52R, or P53G, or a combination thereof. In some instances, the Fe region includes L234A and L235A in combination with K46G, L52R, or P53G. In some cases, the Fc region includes L234A, L235A, and K46G. In some cases, the Fe region includes L234A. L235A, and L52R. In some cases, the Fe region includes L234A, L235A, and P53G. In some cases, the Fc region includes D265A and N21G. In some cases, the residue position is in reference to IgG1.

In some embodiments, the Fe region includes a mutation at residue position L233, L234, D264, N20, K45, L51, or P52. In some instances, the Fe region includes mutations at L233 and L234 in combination with a mutation at residue position K45, L51, or P52. In some cases, the Fc region includes mutations at L233, L234, and K45. In some cases, the Fe region includes mutations at L233, L234, and L51. In some cases, the Fe region includes mutations at L233, L234, and K45. In some cases, the Fc region includes mutations at L233, L234, and P52. In some instances, the Fc region includes mutations at D264 and N20. In some cases, equivalent positions to residue L233, L234, D264, N20, K45, L51, or P52 in an IgG1, IgG2, IgG3, or IgG4 framework are contemplated.

n some embodiments, the Fc region includes L233A, L234A, D264A, N20G, K45G, L51R, or P52G. In some instances, the Fc region includes L233A and L234A. In some instances, the Fe region includes L233A and L234A in combination with K45G, L51R, or P52G. In some cases, the Fe region includes L233A, L234A, and K45G. In some cases, the Fc region includes L233A, L234A, and L51R. In some cases, the Fe region includes L233A, L234A, and K45G. In some cases, the Fe region includes L233A, L234A, and P52G. In some instances, the Fc region includes D264A and N20G. In some cases, the residue position is in reference to IgG1.

In some embodiments, the human IgG constant region is modified to alter antibody-dependent cellular cytotoxicity (ADCC) and/or complement-dependent cytotoxicity (CDC), e.g., with an amino acid modification described inNatsume et al., 2008 Cancer Res, 68(10): 3863-72; Idusogie et al., 2001 J Immunol, 166(4): 2571-5; Moore et al., 2010 mAbs, 2(2): 181-189; Lazar etal, 2006 PNAS, 103(11): 4005-4010, Shields etal, 2001 JBC, 276(9): 6591-6604; Stavenhagen etal., 2007 Cancer Res, 67(18): 8882-8890; Stavenhagen etal., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the antibody of IgG1, IgG2, IgG3 or IgG4 is human or humanized antibody. The information of IgG1, IgG2, IgG3 or IgG4 can be obtained on NCBI or UniProt (https://www.uniprot.org/).

In some embodiments of the present application, the antibody is bispecific antibodies. In some embodiments of the present application, the antibody is IgG1 like bispecific antibodies.

In some embodiments of the present application, those skilled in the art can select suitable method to prepare the bispecific antibodies. In some embodiments of the present application, the bispecific antibodies can be obtained by Knobs-in-holes technology (Ridgway J B B, Presta L G, Paul C. ‘Knobs-into-holes’ engineering of antibody CH3 domains for heavy chain heterodimerization[J]. Protein Engineering (7):617(2023-08-11).), format chain exchange (FORCE) technology, a common light chain format technology (De Nardis C, Hendriks L J A, Poirier E, et al. A new approach for generating bispecific antibodies based on a common light chain format and the stable architecture of human immunoglobulin GI [J]. Journal of Biological Chemistry, 2017:jbc.M117.793497.), controlled Fab arm exchange technology (Yanakieva De, Pekar L, Evers A, et al. Beyond bispecificity: Controlled Fab arm exchange for the generation of antibodies with multiple specificities[J]. MABS, 2022, 14(1), e2018960), CrossMAb technology (Klein C, Schaefer W, Regula J T. The use of CrossMAb technology for the generation of bi- and multispecific antibodies[J]. MABS, 2016, 8(6), P1010-P1020.) or their combination.

As used herein, the term “knobs-into-holes” is used in its broadest sense and encompasses various situations, such as the CH1 domain of one heavy chain with the knob mutations and the CH1 domain of the other heavy chain with the hole mutations, the CH2 domain of one heavy chain with the knob mutations and the CH2 domain of the other heavy chain with the hole mutations, and/or the CH3 domain of one heavy chain with the knob mutations and the CH3 domain of the other heavy chain with the hole mutations. For example, and generally, “knobs-into-holes” may refer to an intra-interface modification between two antibody heavy chains in the CH3 domains: i) in the CH3 domain of one heavy chain (first CH3 domain), an amino acid residue is substituted with another amino acid residue bearing a large side chain, thereby creating a protrusion (“knob”) in the interface in the first CH3 domain; ii) in the CH3 domain of the other heavy chain (second CH3 domain), an amino acid residue is substituted with another amino acid residue bearing a smaller side chain, thereby creating a cavity (“hole”) within the interface in the second CH3 domain, in which a protrusion (“knob”) in the first CH3 domain can be placed.

In some embodiments of the present application, the antibody is selected from any one of cytotoxic antibodies, inhibitors of cell proliferation, regulators of cell activation and interaction, regulators of the human immune system, neutralizations of antigens, antibodies that are immunospectific for viral antigens or antibodies that are immunospectific for microbial antigens.

There is no specific limitation to the antibody. According to the antigens associated with the disease, those skilled in the art can select suitable antibody useful in the bio-conjugation process of the present application.

In some embodiments, the antibody is target-specific, which is targeted to, HER2 (Human Epidermal GrowthFactor Receptor 2), TROP2 (TACSTD2, tumor associated calcium signal transducer 2), BCMA (TNFRSF17, TNF receptor superfamily member 17).

In some embodiments, the antibody is Trastuzumab, Sacituzumab or Belantamab.

In some embodiments, the antibody can be obtained commercially or produced by any method known to those skilled in the art.

The present application provides a modification prepared by the method described above.

The resultant modified antibody includes ADC. In some embodiments, the ADC comprises the ADC with D2, the ADC with D4, the ADC with D1, the ADC with D6, the ADC with D3, the ADC with D1+D6, the ADC with D1+D2, the ADC with D1+D4, the ADC with D2+D4, the ADC with D6+D2, the ADC with D6+D1, the ADC with D3+D1, the ADC with D3+D2, the ADC with D0+D6, or the ADC with D0+D2.

In some embodiments, the ADC is Trastuzumab-[MC-VC-PAB-MMAE]₆, Sacituzumab-[MC-VC-PAB-MMAE]₆, Belantamab-[MC-VC-PAB-MMAE]₆, Trastuzumab-[MC-VC-PAB-MMAE]₆ [MC-GGFG-DXd]₂, Trastuzumab-[MC-GGFG-DXd]₆[Maleimide-PEG4-N3-DBCO-Cy3]₁, Trastuzumab-[Maleimide]₆[MC-VC-PAB-MMAE]₂, Trastuzumab-[Maleimide]₆[Maleimide-PEG4-N3-DBCO-Cy3]₁, Trastuzumab-[MC-VC-PAB-MMAE]₂, Trastuzumab-[Maleimide-PEG4-N3-DBCO-Cy3]₁, Trastuzumab-[Maleimide-PEG4-N3-DBCO-Cy3]₁[MC-VC-PAB-MMAE]₆, Trastuzumab-[Maleimide-PEG4-N3-DBCO-Cy3]₁[MC-VC-PAB-MMAE]₂, Trastuzumab-[Maleimide-PEG4-N3-DBCO-Cy3]₁[MC-VC-PAB-MMAE]₄ and Trastuzumab-[MC-GGFG-DXd]₂[MC-VC-PAB-MMAE]₄.

The disclosure also provides the antibody with site-specific modification, of which two interchain disulfide bonds in the Fab region and one interchain disulfide bond in the hinge region of the antibody are reduced, conjugated, or modified.

In some embodiments, the antibody with site-specific modification (ADC with D6) is prepared by the method includes the step (A1) and (B1), wherein the first payload units are the first linker-payloads.

In some embodiments, the antibody with site-specific modification (ADC with D3) is prepared by the method including the step (A1) and (B1), wherein the first payload units are the first thio-bridging reagent bearing reactive groups which reacts with the first linker-payload.

In some embodiments, the antibody with site-specific modification (ADC with D3) is prepared by the method including the step (A1) and (B1), wherein the first payload units are the first thio-bridging reagent bearing the first linker-payloads.

The disclosure also provides the antibody with site-specific modification, of which one interchain disulfide bonds in the hinge region of the antibody are reduced, conjugated, or modified.

In some embodiments, provided herein is the antibody with site-specific modification (ADC with D2) prepared by the method including the step (A1), (A2), and (B1), wherein the first payload units are the first linker-payloads.

In some embodiments, the antibody with site-specific modification (ADC with D1) is prepared by the method including the step (A1), (A2) and (B1), wherein the first payload units are the first thio-bridging reagent bearing reactive groups which reacts with the first linker-payload.

In some embodiments, the antibody with site-specific modification (ADC with D1) is prepared by the method including the step (A1), (A2) and (B1), wherein the first payload units are the first thio-bridging reagent bearing the first linker-payloads.

The disclosure also provides the antibody with site-specific modification, of which two interchain disulfide bonds in the hinge region of the antibody are reduced, conjugated, or modified.

In some embodiments, the antibody with site-specific modification (ADC with D4) is prepared by the method including the step (A1), (A2), (A3), and (B1), wherein the first payload units are the first linker-payloads.

In some embodiments, provided herein is the antibody with site-specific modification (ADC with D1+D2) prepared by the method including the step (A1), (A2), (B1), (B2) and (B3), wherein the first payload units are the first thio-bridging reagent bearing the first linker-payloads, and the second payload units are the second linker-payloads. Meanwhile, the transition metal ions are introduced in step (B2).

In some embodiments, provided herein is the antibody with site-specific modification (ADC with D1+D2) prepared by the method including the step (A1), (A2), (B1), (B2) and (B3), wherein the first payload units are the first thio-bridging reagent bearing reactive groups which reacts with the first linker-payloads, and the second payload units are the second linker-payloads. Meanwhile, the transition metal ions are introduced in step (B2).

In some embodiments, provided herein is the antibody with site-specific modification (ADC with D2+D2) prepared by the method including the step (A1), (A2), (B1), (B2) and (B3), wherein the first payload units are the first linker-payloads, and the second payload units are the second linker-payloads. Meanwhile, the transition metal ions are introduced in step (B2).

In some embodiments, provided herein is the antibody with site-specific modification (ADC with D2+D1) prepared by the method including the step (A1), (A2), (B1), (B2) and (B3), wherein the first payload units are the first linker-payloads, and the second payload units are the second thio-bridging reagent bearing the second linker-payloads. Meanwhile, the transition metal ions are introduced in step (B2).

In some embodiments, provided herein is the antibody with site-specific modification (ADC with D2+D1) prepared by the method including the step (A1), (A2), (B1), (B2) and (B3), wherein the first payload units are the first linker-payloads, and the second payload units are the second thio-bridging reagent bearing reactive groups which reacts with the second linker-payloads. Meanwhile, the transition metal ions are introduced in step (B2).

In some embodiments, provided herein is the antibody with site-specific modification (ADC with D1+D4) prepared by the method including the step (A1), (A2), (B1), (B2) and (B3), wherein the first payload units are the first thio-bridging reagent bearing the first linker-payloads, and the second payload units are the second linker-payloads. Meanwhile, the transition metal ions are introduced in step (B2).

In some embodiments, provided herein is the antibody with site-specific modification (ADC with D1+D4) prepared by the method including the step (A1), (A2), (B1), (B2) and (B3), wherein the first payload units are the first thio-bridging reagent bearing reactive groups which reacts with the first linker-payloads, and the second payload units are the second linker-payloads. Meanwhile, the transition metal ions are introduced in step (B2).

In some embodiments, provided herein is the antibody with site-specific modification (ADC with D2+D4) prepared by the method including the step (A1), (A2), (B1), (B2) and (B3), wherein the first payload units are the first linker-payloads, and the second payload units are the second linker-payloads. Meanwhile, the transition metal ions are introduced in step (B2).

The disclosure also provides the antibody with site-specific modification, of which two interchain disulfide bonds in the Fab region and two interchain disulfide bonds in the hinge region of the antibody are reduced, conjugated, or modified.

In some embodiments, provided herein is the antibody with site-specific modification (ADC with D6+D2) prepared by the method including the step (A1), (B1), (B2) and (B3), wherein the first payload units are the first linker-payloads, and the second payload units are the second linker-payloads.

In some embodiments, provided herein is the antibody with site-specific modification (ADC with D6+D1) prepared by the method including the step (A1), (B1), (B2) and (B3), wherein the first payload units are the first linker-payloads, and the second payload units are the second thio-bridging reagent bearing reactive groups which reacts with the second linker-payloads.

In some embodiments, provided herein is the antibody with site-specific modification (ADC with D6+D1) prepared by the method including the step (A1), (B1), (B2) and (B3), wherein the first payload units are the first linker-payloads, and the second payload units are the second thio-bridging reagent bearing the second linker-payloads.

In some embodiments, provided herein is the antibody with site-specific modification (ADC with D3+D2) prepared by the method including the step (A1), (B1), (B2) and (B3), wherein the first payload units are the first thio-bridging reagent bearing the first linker-payloads, and the second payload units are the second linker-payloads.

In some embodiments, provided herein is the antibody with site-specific modification (ADC with D3+D2) prepared by the method including the step (A1), (B1), (B2) and (B3), wherein the first payload units are the first thio-bridging reagent bearing reactive groups which reacts with the first linker-payloads, and the second payload units are the second linker-payloads.

In some embodiments, provided herein is the antibody with site-specific modification (ADC with D3+D1) prepared by the method includes the step (A1), (B1), (B2) and (B3), wherein the first payload units are the first thio-bridging reagent bearing the first linker-payloads, and the second payload units are the second thio-bridging reagent bearing the second linker-payloads.

In some embodiments, provided herein is the antibody with site-specific modification (ADC with D3+D1) prepared by the method includes the step (A1), (B1), (B2) and (B3), wherein the first payload units are the first thio-bridging reagent bearing reactive groups which reacts with the first linker-payloads, and the second payload units are the second thio-bridging reagent bearing reactive groups which reacts with the second linker-payloads.

In some embodiments, provided herein is the antibody with site-specific modification (ADC with D0+D2) prepared by the method including the step (A1), (B1), (B2) and (B3), wherein the first payload units are the first thio-bridging reagent, and the second payload units are the second linker-payloads.

In some embodiments, provided herein is the antibody with site-specific modification (ADC with D0+D1) prepared by the method includes the step (A1), (B1), (B2) and (B3), wherein the first payload units are the first thio-bridging reagent, and the second payload units are the second thio-bridging reagent bearing the second linker-payloads.

In some embodiments, provided herein is the antibody with site-specific modification (ADC with D0+D1) prepared by the method includes the step (A1), (B1), (B2) and (B3), wherein the first payload units are the thio-bridging reagent, and the second payload units are the second thio-bridging reagent bearing reactive groups which reacts with the second linker-payloads.

In some embodiments, provided herein is the antibody with site-specific modification (ADC with D2+D6) prepared by the method including the step (A1), (A2), (B1), (B2) and (B3), wherein the first payload units are the first linker-payloads, and the second payload units are the second linker-payloads.

In some embodiments, provided herein is the antibody with site-specific modification (ADC with D1+D6) prepared by the method including the step (A1), (A2), (B1), (B2) and (B3), wherein the first payload units are the first thio-bridging reagent bearing reactive groups which reacts with the first linker-payloads, and the second payload units are the second linker-payloads.

In some embodiments, provided herein is the antibody with site-specific modification (ADC with D1+D6) prepared by the method including the step (A1), (A2), (B1), (B2) and (B3), wherein the first payload units are the first thio-bridging reagent bearing the first linker-payloads, and the second payload units are the second linker-payloads.

In some embodiments, provided herein is the antibody with site-specific modification (ADC with D2+D3) prepared by the method including the step (A1), (A2), (B1), (B2) and (B3), wherein the first payload units are the first linker-payloads, and the second payload units are the second thio-bridging reagent bearing the second linker-payloads.

In some embodiments, provided herein is the antibody with site-specific modification (ADC with D2+D3) prepared by the method including the step (A1), (A2), (B1), (B2) and (B3), wherein the first payload units are the first linker-payloads, and the second payload units are the second thio-bridging reagent bearing reactive groups which reacts with the second linker-payloads.

In some embodiments, provided herein is the antibody with site-specific modification (ADC with D1+D3) prepared by the method includes the step (A1), (A2), (B1), (B2) and (B3), wherein the first payload units are the first thio-bridging reagent bearing the first linker-payloads, and the second payload units are the second thio-bridging reagent bearing the second linker-payloads.

In some embodiments, provided herein is the antibody with site-specific modification (ADC with D1+D3) prepared by the method includes the step (A1), (A2), (B1), (B2) and (B3), wherein the first payload units are the first thio-bridging reagent bearing reactive groups which reacts with the first linker-payloads, and the second payload units are the second thio-bridging reagent bearing reactive groups which reacts with the second linker-payloads.

In some embodiments, provided herein is the antibody with site-specific modification (ADC with D0+D6) prepared by the method including the step (A1), (A2), (B1), (B2) and (B3), wherein the first payload units are the first thio-bridging reagent, and the second payload units are the second linker-payloads.

In some embodiments, provided herein is the antibody with site-specific modification (ADC with D0+D3) prepared by the method includes the step (A1), (A2), (B1), (B2) and (B3), wherein the first payload units are the first thio-bridging reagent, and the second payload units are the second thio-bridging reagent bearing the second linker-payloads.

In some embodiments, provided herein is the antibody with site-specific modification (ADC with D0+D3) prepared by the method includes the step (A1), (A2), (B1), (B2) and (B3), wherein the first payload units are the thio-bridging reagent, and the second payload units are the second thio-bridging reagent bearing reactive groups which reacts with the second linker-payloads.

Various analytical methods can be used to determine the yields and isomeric mixtures of the antibody with site-specific modification. In some embodiments of the present application, the analytical method is HIC-HPLC. HIC-HPLC can separate the antibodies loaded with various numbers of linker-payload. The loading level of payload can be determined based on the ratio of absorbances, e.g., at 250 nm and 280 nm. For example, if a payload (e.g., drug) can absorb at 250 nm while the antibody absorbs at 280 nm. The 250/280 ratio therefore increases with drug loading.

The process of generating antibodies with site-specific modification bypasses any need of protein engineering or enzyme catalysis, but is based on native inter-chain disulfide bonds, and only needs novel reductants and transition metal ions. Therefore, the process of the disclosure is less complicate, the homogeneity of the resultant antibodies with site-specific modification (antibody-drug conjugate) is dramatically improved.

### Pharmaceutical Composition

The present application also provides a pharmaceutical composition comprising the antibody with site-specific modification prepared by the method described above and at least a pharmaceutically acceptable ingredient.

Pharmaceutical compositions provided herein may be formulated in any manner known in the art, such as, pharmaceutical compositions provided herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that include one or more antibodies or antigen-binding fragments thereof as disclosed herein and one or more antioxidants such as methionine.

In some embodiments, the pharmaceutical compositions can be a liquid solution, suspension, or emulsion. In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments of the present application, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments of the present application, the anti-cancer agents can include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, capmtothecin, topotecan, bryostatin, adezelesin, anthracyclin, carzelesin, bizelesin, dolastatin, auristatins, duocarmycin, eleutherobin, taxols such as paclitaxel or docetaxel, cyclophasphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, epirubicin, blcomycin, plicamycin, mitomycin and the like.

The disclosure provides the use of the antibody with site-specific modification provided herein in the manufacture of a therapeutic agent for preventing, diagnosing or treating a disease.

As use herein, the term “treat” of any disease refers to alleviating or ameliorating the disease (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent” of any disease refers to the prophylactic treatment of the disease; or delaying the onset or progression of the disease.

In some embodiments, the disease is a tumor or cancer. In some embodiments, the disease is an autoimmune disease and the like.

In some embodiments of the present application, the cancer can include, but not limited to, carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The disclosure provides the method of preventing, diagnosing or treating a disease in a subject in need thereof, comprising administrating to the subject a therapeutically effective amount of the antibody with site-specific modification prepared by the method described above.

As use herein, the term “subject” refers to mammals, primates (e.g., humans, male or female), dogs, rabbits, guinea pigs, pigs, rats and mice. In certain embodiments, the subject is a primate. In yet other embodiments, the subject is a human.

As used herein, the term “a therapeutically effective amount” refers to an amount of the ADC of the present application that will elicit the biological or medical response of a subject, for example, ameliorate symptoms, alleviate conditions, slow or delay disease progression, or prevent a disease, etc. The therapeutically effective amount will vary with the type and severity of the condition to be alleviated. It is to be further understood that for any particular subject, specific dosage regimens should be adjusted over time according to the individual need and the professional judgment of the person administering or supervising the administration of the compositions. In some embodiments of the present application, the therapeutically effective amount is based on a variety of factors, such as the type of disease, the age, weight, sex, medical condition of the patient, the severity, of the condition, the route of administration, and the particular antibody employed. In some embodiments of the present application, the therapeutically effective amount can vary widely, but can be determined routinely using standard methods. In some embodiments of the present application, the therapeutically effective amount can be adjusted based on the pharmacokinetic or pharmacodynamic parameters, which may include clinical effects such as toxic effects and/or laboratory values.

It will be readily apparent to those skilled in the art that other suitable modifications and adaptations of the present application described herein are obvious and may be made using suitable equivalents without departing from the scope of the disclosure or the embodiments disclosed herein. Having now described the disclosure in detail, the same will be more clearly understood by reference to the following examples, which are included for purposes of illustration only and are not intended to be limiting. Further, unless specifically described otherwise, the reagent and the solvent described in the description can be easily obtained from a commercial supplier.

### EXAMPLES

The invention is further described in the following examples, which do not limit the scope of the invention described in the claims.

### Reagent and Manufacturer

Trastuzumab is commercially available from Roche.

Sacituzumab and Belantamab are commercially available from MedChemExpress.

EDTA is commercially available from Aladdin.

DMA (Dimethylacetamide) is commercially available from Aldrich Sigma.

MC-VC-PAB-MMAE is commercially available from Levena biopharma.

MC-GGFG-DXd is commercially available from Levena biopharma.

Desalting column (type: 40K, 0.5 mL, REF:87766, Lot SJ251704) is commercially available from Thermo Scientific.

DBCO-Cy3 is commercially available from Confluore.

TCEP is commercially available from Bidepharm.

Dibromomalcimide is commercially available from Aladdin.

The reagents used in examples, include but not limited to 1-(3-Dimethylaminopropyl)-3-ethylcarbodiimide hydrochloride (EDC), 1-Hydroxybenzotriazole (HOBt), N,N-Diisopropylethylamine (DIPEA), ethyl acetate (EtOAc), N,N-Dimethylformamide (DMF), Bicyclic amidine (DBU), 2-(7-Azabenzotriazol-1-yl)-N,N,N′,N′-tetramethyluronium hexafluorophosphate (HATU), N-(3-dimethylaminopropyl)-N′-ethylcarbodiimide hydrochloride (EDCI), trifluoroacetic acid (TFA), dichloromethane (DCM), tert-butylchlorodiphenylsilane (TBDPSCl) are commercially available.

### Synthesis Procedure a of the Compound According to the Present Application

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and the amine reagent (0.5 mmol, depending on the structure of the compound) was added.

The reaction was stirred at room temperature for 1 h. The reaction mixture was purified by RP-HPLC using a C18 column yielding the desired product.

### Synthesis Procedure A-1 of the Compound According to the Present Application

The product prepared by the synthesis procedure A was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Synthesis Procedure B of the Compound According to the Present Application

To a solution of TCEP (286.6 mg. 1.0 mmol, 2.0 eq.) in DMF (3 mL) was added HOBt (67.5 mg, 0.5 mmol, 1 eq) and EDCI (95.5 mg, 0.5 mmol, 1.0 eq), followed by DIPEA (2.0 mmol, 4.0 eq) under N₂ atmosphere. The mixture was stirred for 30 min and the amine reagent (0.5 mmo, depending on the structure of the compound) was added. The reaction was stirred at room temperature for 1 h. The reaction mixture was purified by RP-HPLC using a C18 column yielding the corresponding product.

### Synthesis Procedure B-1 of the Compound According to the Present Application

The product prepared by the synthesis procedure B was dissolved in DCM (3 mL) and TFA (0.3 mL) was added. The mixture was stirred for 1 h, LCMS showed reaction was completed. The mixture was concentrated and the residue was taken up by distilled water, washed with EtOAc twice. The aqueous layer was lyophilized to give corresponding product.

### Homogeneity Assays

The drug/antibody ratio (DAR) and product distribution were analyzed using HIC-HPLC (Agilent1200) with a TSK gel Butyl-NPR column (2.5 μm, 4.6 mm*3.5 cm) (commercially available from Tosoh Biosciences) at a flow rate of 0.5 mL/min at 25° C. Solvent A was 1.5 M (NH₄)₂SO₄ and 50 mM K₂HPO₄ 3H₂O. Solvent B was 75% v/v 21.3 mM KH₂PO₄, 28.6 mM K₂HPO₄ 3H₂O and 25% v/v isopropanol. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

### Example 1: Synthesis of TCEP-NO-Trtyl and TCEP-NO

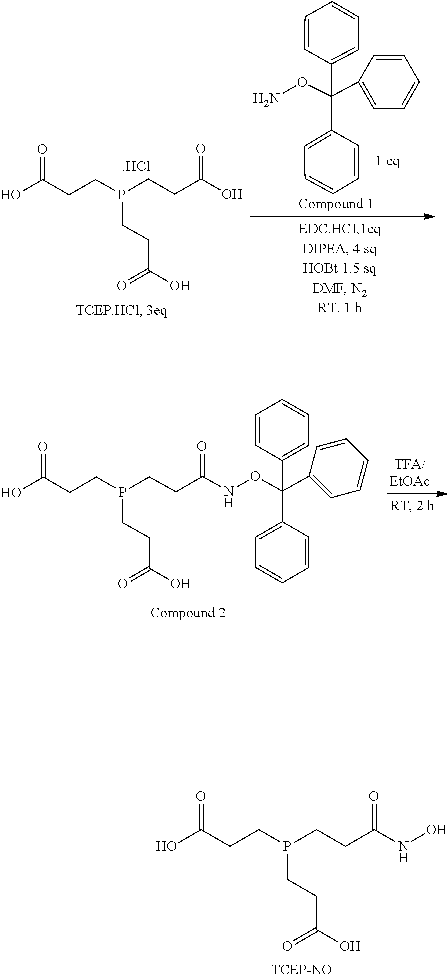 [SMILES: O=C(O)CCP(CCC(=O)O)CCC(=O)O ; ".HCl" ; NOC(c1ccccc1)(c1ccccc1)c1ccccc1 ; O=C(O)CCP(CCC(=O)O)CCC(=O)NOC(c1ccccc1)(c1ccccc1)c1ccccc1 ; O=C(O)CCP(CCC(=O)O)CCC(=O)NO]

Compound 1 O-trithylhydroxylamine (NH₂—O-Trt, 176 μmol, 48.5 mg, 1 eq.), EDC (176 μmol 33.7 mg, 1 eq.) and HOBt (352 μmol, 53.8 mg, 2 eq.) were dissolved in 1.5 mL DMF (degassed) under an inert atmosphere. This solution was added to TCEP (528 mol, 150 mg, 3 eq.) dissolved in 1.5 mL degassed DMF containing DIPEA (704 μmol, 123 uL, 4 eq.) under an inert atmosphere. The reaction was stirred at room temperature for 60 min. After that, DMF was removed in vacuo and the residue was added CH₃COOH (0.1 N, 5 ml) and EtOAc (2 ml). The resulting mixture was stirred for 5 min and filtered to yield Compound 2 (TCEP-NO-Trtyl) as a white solid (61.2 mg, 12.2%). For Compound 2 (also called TCEP-NO-Trtyl, or TCEP-19-int1), MS[M−H]⁻=506.2, Exact mass calc. for C₂₈H₃₀NO₆P is 507.18. ¹H NMR (400 MHz, DMSO-d6): δ 7.40-7.15 (m, 15H), 2.48-2.38 (m, 4H), 2.22 (s, 1H), 2.12-1.52 (m, 7H).

Without further purification, Compound 2 was added EtOAc (2 ml), 5% TFA and 5% TIPS. The reaction was continued for another 2 h. Then H₂O (5 ml) and EtOAc (10 ml) were introduced. The aqueous phase was futher washed with EtOAc (10 ml) twice and concentrated to afford TCEP-NO (13 mg). For TCEP-NO, MS[M−H]⁻=263.94, exact mass calc. for C₉H₁₆NO₆P is 265.07. ¹H-NMR (400 MHz, Deuterium Oxide): δ 2.93-2.85 (m, 4H), 2.73-2.56 (m, 8H).

### Example 2: Synthesis of TCEPA

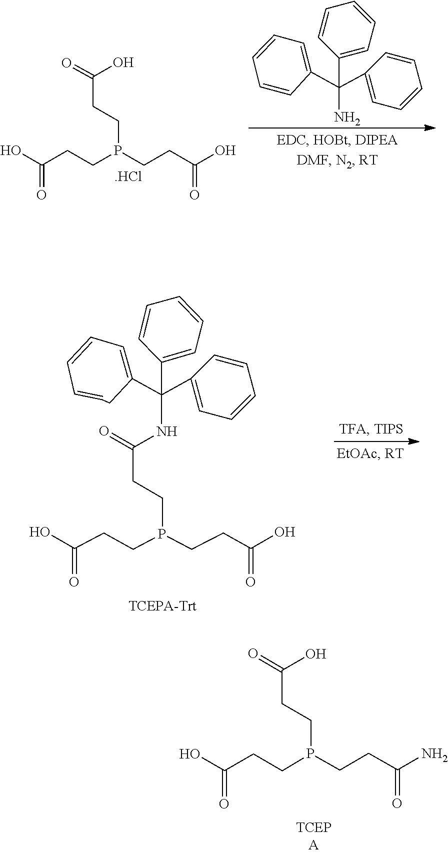 [SMILES: O=C(O)CCP(CCC(=O)O)CCC(=O)O ; ".HCl" ; NC(c1ccccc1)(c1ccccc1)c1ccccc1 ; O=C(O)CCP(CCC(=O)O)CCC(=O)NC(C1=CC=CC=CC1)(c1ccccc1)c1ccccc1 ; NC(=O)CCP(CCC(=O)O)CCC(=O)O]

Tritylamine (176 μmol, 45.6 mg, 1 eq.), EDC (176 μmol 33.7 mg, 1 eq.) and IIOBt (352 μmol, 53.8 mg, 2 eq.) were dissolved in 1.5 mL DMF (degassed) under an inert atmosphere. This solution was added to TCEP (528 μmol, 150 mg, 3 eq.) dissolved in 1.5 mL degassed DMF containing DIPEA (704 μmol, 123 ul, 4 eq.) under an inert atmosphere. The reaction was stirred at room temperature for 60 min. After that, DMF was removed in vacuo and the residue was added CH₃COOH (0.1 N, 5 ml) and EtOAc (20 ml). The resulting mixture was washed with H2O three times and the organic phase was concentrated followed by petroleum ether precipitation to yield TCEPA-Trt as a white solid, without further purification which was added EtOAc (2 ml), 5% TFA and 5% TIPS. The reaction was continued for another 2 h. Then H₂O (5 ml) and EtOAc (10 ml) were introduced. The aqueous phase was further washed with EtOAc (10 ml) twice and concentrated to afford TCEPA (8 mg). MS[M+H]⁺=250.18, exact mass calc. for C₉H₁₆NO₅P is 249.08. ¹H NMR (400 MHz, Deuterium Oxide) δ 2.85-2.70 (m, 4H), 2.61-2.43 (m, 6H), 2.15-2.06 (m, 2H).

### Example 3: Synthesis of TCEP-1

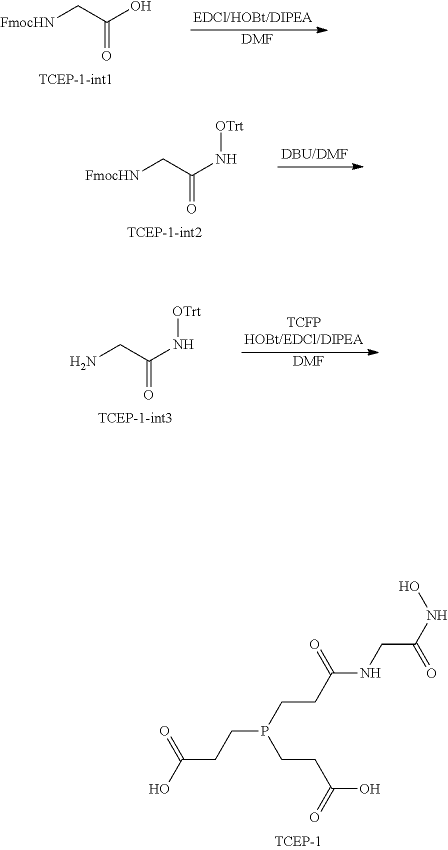

1. TCEP-1-int2

To a solution of TCEP-1-int1 (3.0 g, 10.0 mmol, 1.0 eq, Fmoc-Glycine, Bidepharm) in DMF (30 mL) was added HOBt (1.64 g, 12.0 mmol, 1.2 eq) and EDCI (2.32 g, 12 mmol, 1.2 eq), followed by DIPEA (4.4 mL, 25.2 mmol, 2.5 eq) under N2 atmosphere. The mixture was stirred for 30 min and the Compound 1 O-Trithylhydroxylamine (2.78 g, 10 mmol, 1.0 eq, Bidepharm) was added. The reaction mixture was stirred at room temperature for 1 h and poured into ice-water. The precipitate was collected by filtration and washed with water. The filter cake was dried over vacuum to give the crude product TCEP-1-int2 (4.5 g, 80% yield, white solid), which was used in next step directly without further purification.

2. TCEP-1-int3

To a solution of TCEP-1-int2 (4.5 g, 8.1 mmol, 1.0 eq) in DMF (25 mL) was added DBU (5 mL). The resulting mixture was stirred for 0.5 h at room temperature, LCMS showed reaction was completed. The mixture was poured into ice-water, and extracted with EtOAc, dried over vacuum and purified with flash column (EtOAc/petroleum ether=0-50%) to give product TCEP-1-int3 (2.0 g, 77% yield, white solid).

### 3. TCEP-1

TCEP-1 was synthesized as the synthesis procedure B-1 wherein TCEP-1-int3 was the amine reagent, yielding TCEP-1 (45.1 mg, 28%) as white solid. MS[M−H]⁻=321.15, exact mass calc. for C₁₁H₁₉N₂O₇P is 322.25. ¹H-NMR (400 MHz, Deuterium Oxide): δ 3.99 (s, 0.64H), 3.87 (s, 1.34H), 2.96-2.81 (m, 6H), 2.63-2.56 (m, 6H).

### Example 4: Synthesis of TCEP-2

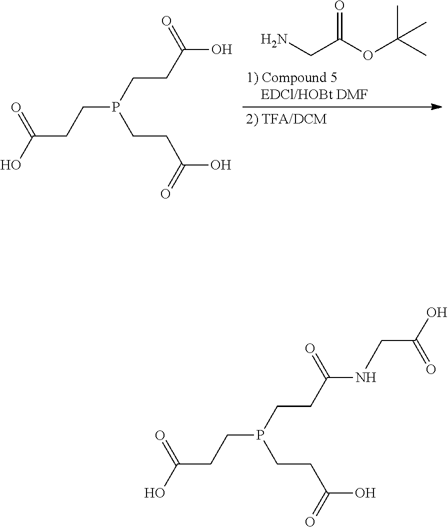

TCEP-2 was synthesized as the synthesis procedure B-1 wherein the Compound 5 (tert-Butyl glycinate, Bidepharm) was the amine reagent, yielding TCEP-2 (52.3 mg, 34%) as white solid. MS[M−H]⁻=306.18, exact mass calc. for C₁₁H₁₈NO₇P is 307.24. ¹H NMR (400 MHz, Deuterium Oxide): δ 3.99 (s, 2H), 3.00-2.76 (m, 6H), 2.60 (dtd, J=14.0, 7.0, 3.8 Hz, 6H).

### Example 5: Synthesis of TCEP-3

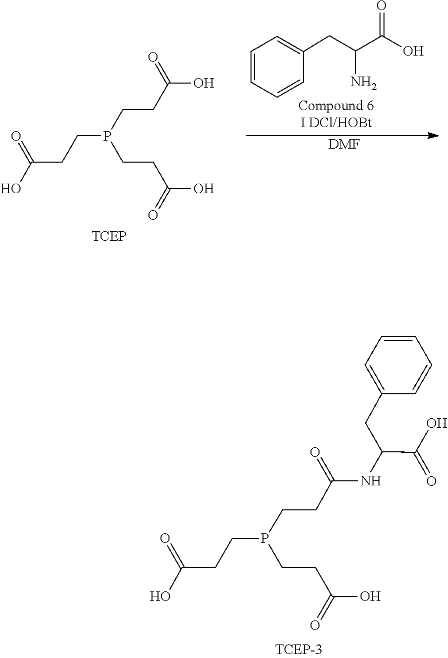

TCEP-3 was synthesized as the procedure B wherein the compound 6 (DL-Phenylalanine, Adamas) is amine reagent, yielding TCEP-3 (65.0 mg, 33%) as white solid. MS[M−H]−=396.24, exact mass calc. for C₁₈H₂₄NO₇P is 397.13. ¹H NMR (400 MHz, Deuterium Oxide): δ 7.43-7.25 (m, 5H), 4.71 (dd, J=10.3, 4.8 Hz, 1H), 3.31 (dd, J=13.9, 4.8 Hz, 1H), 2.91 (dd, J=13.9, 10.3 Hz, 1H), 2.86-2.59 (m, 6H), 2.53-2.27 (m, 6H).

### Example 6: Synthesis of TCEP-4

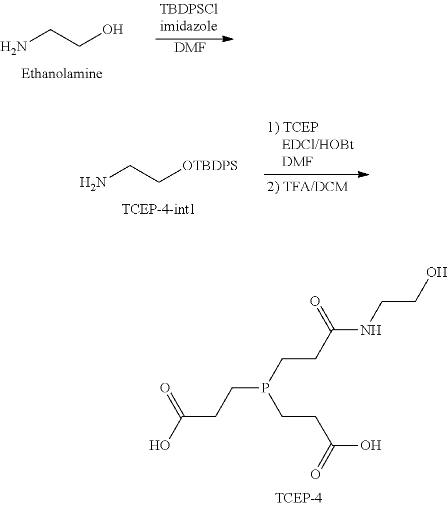

1. TCEP-4-int1

To a solution of Ethanolamine (610 mg, 10 mmol, 1.0 eq, Adamas) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-4

TCEP-4 was synthesized as the synthesis procedure B-1 wherein TCEP-4-int1 was amine reagent, yielding TCEP-4 (60.0 mg, 40.8%) as white solid. MS[M−H]−=292.25, exact mass calc. for C₁₁H₂₁NO₆P is 293.10. ¹H NMR (400 MHz, Deuterium Oxide): δ 4.23 (t, J=5.4 Hz, 1H), 3.64 (t, J=5.4 Hz, 1H), 3.48 (t, J=5.3 Hz, 1H), 3.32 (t, J=5.5 Hz, 1H), 2.98-2.78 (m, 6H), 2.60 (dp, J=13.4, 6.8 Hz, 6H).

### Example 7: Synthesis of TCEP-5

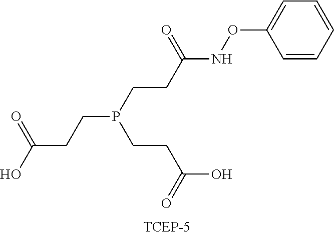 [SMILES: O=C(O)CCP(CCC(=O)O)CCC(=O)NOc1ccccc1]

TCEP-5 was synthesized as the procedure B wherein (2-phenoxy-ethylamine, Bidepharm) was amine reagent, yielding TCEP-5 (105.0 mg, 56.8%) as white solid. MS[M−H]⁻=368.24, exact mass calc. for C₁₇H₂₄NO₆P is 369.13. ¹H NMR (400 MHz, Deuterium Oxide): δ 7.34 (dd, J=8.5, 7.2 Hz, 2H), 7.00 (dd, J=19.2, 7.7 Hz, 3H), 4.14 (t, J=5.1 Hz, 2H), 3.56 (t, J=5.1 Hz, 2H), 2.78 (ddt, J=41.6, 20.1, 6.9 Hz, 6H), 2.60-2.43 (m, 6H).

### Example 8: Synthesis of TCEP-6

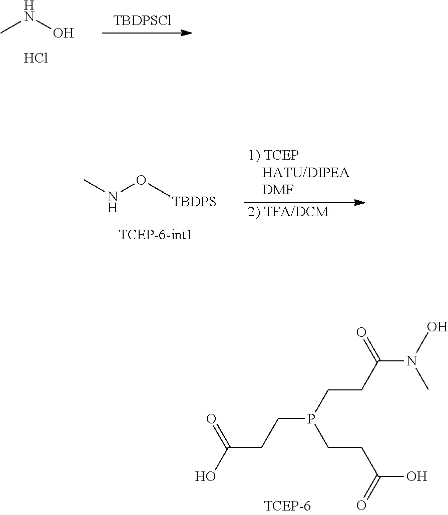

1. TCEP-6-int1

To a solution of N-Methylhydroxylamine hydrochloride (830 mg, 10 mmol, 1.0 eq, Bidepharm) in DMC (20 mL) was added imidazole (15 mmol, 1.5 eq) followed by TBDPSCl (10 mmol, 1.0 eq, Adamas) at 0° C. The mixture was stirred for 2 h at room temperature. TLC showed reaction was completed, the reaction mixture was washed with water and brine, organic layer was dried over Na₂SO₄ and filtered. The filtrate was concentrated to give the crude product, which was used in next step directly without further purification.

### 2. TCEP-6

TCEP-6 was synthesized as the procedure A-1 wherein TCEP-6-int1 was amine reagent, yielding TCEP-6 (13.0 mg, 9.3%) as white solid. MS[M+H]⁺=280.22, exact mass calc. for C₁₀H₁₈NO₆P is 279.09. ¹H NMR (400 MHz, Deuterium Oxide): δ 3.15 (s, 3H), 3.01-2.80 (m, 4H), 2.64-2.45 (m, 6H), 2.17-2.08 (m, 2H).

### Example 9: Synthesis of TCEP-7

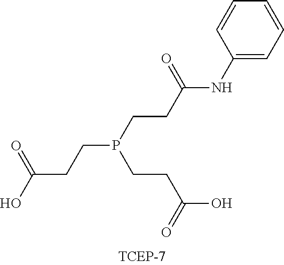 [SMILES: O=C(O)CCP(CCC(=O)O)CCC(=O)Nc1ccccc1]

TCEP-7 was synthesized as the synthesis procedure B wherein (Phenylamine, Adamas) was amine reagent, yielding TCEP-7 (73.0 mg, 45.0% yield) as white solid. MS[M−H]⁻=324.21, exact mass calc. for C₁₅H₂₀NO₅P is 325.11. ¹H NMR (400 MHz, Deuterium Oxide): S 7.42 (d, J=4.3 Hz, 4H), 7.25 (p, J=4.5 Hz, 1H), 2.96 (ddt, J=32.1, 18.3, 6.9 Hz, 6H), 2.75-2.50 (m, 6H).

### Example 10: Synthesis of TCEP-8

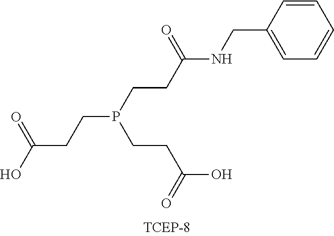 [SMILES: O=C(O)CCP(CCC(=O)O)CCC(=O)NCc1ccccc1]

TCEP-8 was synthesized as the synthesis procedure B wherein (Benzylamine, Adamas) was amine reagent, yielding TCEP-8 (85.6 mg, 50.5% yield) as white solid. MS[M−H]⁻=338.23, exact mass calc. for C₁₆H₂₂NO₅P is 339.12. ¹H NMR (400 MHz, Deuterium Oxide): δ 7.43-7.27 (m, 5H), 4.36 (s, 2H), 2.93-2.77 (m, 6H), 2.63-2.45 (m, 6H).

### Example 11: Synthesis of TCEP-9

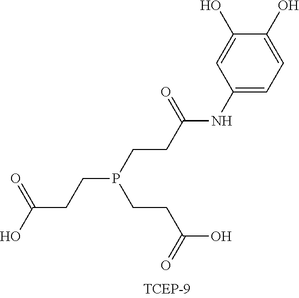 [SMILES: O=C(O)CCP(CCC(=O)O)CCC(=O)Nc1ccc(O)c(O)c1]

TCEP-9 was synthesized as the synthesis procedure A wherein (4-Aminobenzene-1,2-diol, Bidepharm) was amine reagent, yielding TCEP-9 (63.8 mg, 35.7%) as white solid. MS[M−H]=356.20, exact mass calc. for C₁₅H₂₀NO₇P is 357.10. ¹H NMR (400 MHz, Deuterium Oxide): δ 6.97 (d, J=2.4 Hz, 11H), 6.87 (d, J=8.5 Hz, 11H), 6.78 (dd, J=8.5, 2.5 Hz, 11H), 2.92 (ddt, J=17.8, 10.0, 7.0 Hz, 6H), 2.61 (dq, J=13.9, 6.7 Hz, 6H).

### Example 12: Synthesis of TCEP-10

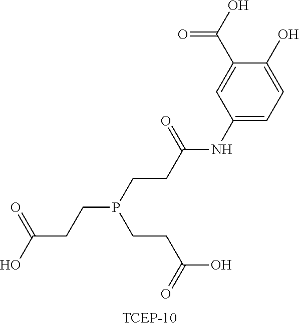 [SMILES: O=C(O)CCP(CCC(=O)O)CCC(=O)Nc1ccc(O)c(C(=O)O)c1]

TCEP-10 was synthesized as the synthesis procedure A wherein (5-Amino-2-hydroxybenzoic acid, Bidepharm) was amine reagent, yielding TCEP-10 (53.7 mg, 27.9%) as white solid. MS[M−H]⁻=384.20, exact mass calc. for C₁₆H₂₀NO₈P is 385.09. ¹H NMR (400 MHz, Deuterium Oxide) δ 7.76 (d, J=2.7 Hz, 1H), 7.41 (dd, J=8.9, 2.7 Hz, 1H), 6.89 (d, J=8.9 Hz, 1H), 3.00-2.83 (m, 6H), 2.61 (dq, J=13.9, 6.7 Hz, 6H).

### Example 13: Synthesis of TCEP-11

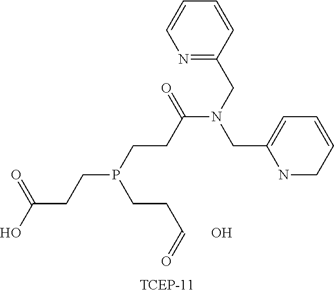

TCEP-11 was synthesized as the synthesis procedure B wherein (Bis(pyridin-2-ylmethyl)amine, Shanghai Acmec Biochemical Co., Ltd) was amine reagent, yielding TCEP-11 (10.5 mg, 4.9%) as brown solid. MS[M+H]⁺=432.24, exact mass calc. for C₂₁HdN₃O₅P is 431.16. ¹H NMR (400 MHz, Deuterium Oxide) δ 8.82-8.68 (m, 2H), 8.58-8.36 (m, 2H), 8.01-7.81 (m, 4H), 5.34 (s, 1H), 5.29 (s, 1H), 5.04 (d, J=2.9 Hz, 2H), 4.48 (s, 1H), 3.16 (dt, J=19.2, 6.5 Hz, 1H), 2.95-2.79 (m, 3H), 2.73-2.48 (m, 5H), 2.20 (ddt, J=36.1, 11.6, 7.5 Hz, 2H).

### Example 14: Synthesis of TCEP-12

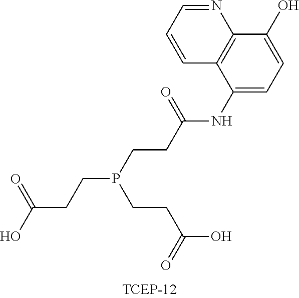 [SMILES: O=C(O)CCP(CCC(=O)O)CCC(=O)Nc1ccc(O)c2ncccc12]

TCEP-12 was synthesized as the synthesis procedure A wherein (5-Amino-8-hydroxyquinoline, Bidepharm) was amine reagent, yielding TCEP-12 (33.2 mg, 16.9%) as white solid. MS[M−H]⁻=391.24, exact mass calc. for C₁₈H₂₁N₂O₆P is 392.11. ¹H NMR (400 MHz, Deuterium Oxide) δ 9.02-8.96 (m, 2H), 8.05-7.98 (m, 1H), 7.84 (s, 1H), 7.64 (d, J=8.4 Hz, 1H), 7.44 (d, J=8.4 Hz, 1H), 2.90-2.80 (m, 2H), 2.67-2.56 (m, 6H), 2.26-2.17 (m, 4H).

### Example 15: Synthesis of TCEP-15

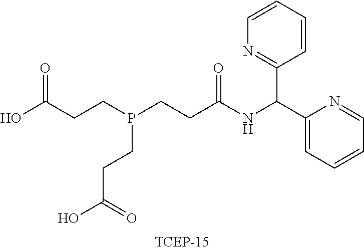 [SMILES: O=C(O)CCP(CCC(=O)O)CCC(=O)NC(c1ccccn1)c1ccccn1]

TCEP-15 was synthesized as the synthesis procedure B wherein (Bis(pyridin-2-yl) methanamine, Bidepharm) was amine reagent, yielding TCEP-15 (21.7 mg, 10.4%) as white solid. MS[M+H]⁺=418.26, exact mass calc. for C₂₀H₂₄N₃O₅P is 417.15. ¹H NMR (400 MHz, Deuterium Oxide) δ 8.69 (td, J=6.2, 1.6 Hz, 2H), 8.37 (dtd, J=15.8, 7.9, 1.7 Hz, 2H), 7.93-7.79 (m, 4H), 3.09-2.72 (m, 6H), 2.70-2.52 (m, 6H).

### Example 16: Synthesis of TCEP-18

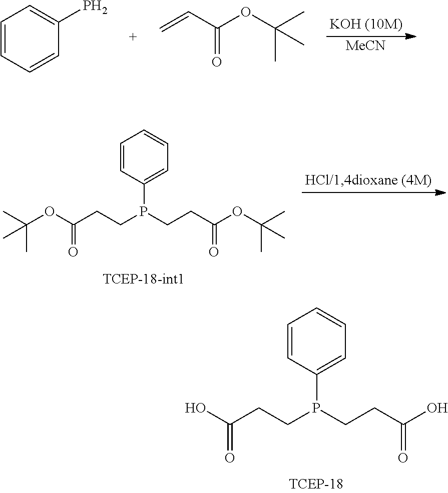

1. TCEP-18-int1

Phenyl phosphine (110 mg, 1.0 mmol, Adamas) was dissolved in acetonitrile (5 ml, degassed) in a flame-dried, round bottom flask under N2(g). Potassium hydroxide (10 N, 10 ul) was added to this mixture, and the resulting solution was cooled to 0° C. Tert-Butyl acrylate (0.44 ml, 3.0 mmol, Adamas) was added. Upon complete addition of Tert-Butyl acrylate, the reaction was heated at 50° C. and stirred for 8 hours. The reaction mixture was taken up by EtOAc (10 mL), then washed with brine (2×5 ml). The organic layer was dried over Na₂SO₄, filtered, and concentrated in vacuo. The residue was purified by flash column (EtOAc/petroleum ether=0-20% (v/v)) to give product TCEP-18-int1 as a clear liquid (254 mg, 69.4%).

### 2. TCEP-18

The solution of TCEP-18-int1 (254 mg, 0.69 mmol) in HCl/1,4-dioxane (4M, Adamas) was stirred for 2 h at room temperature under N₂ atmosphere. LCMS showed reaction was completed and the mixture was concentrated to remove 1,4-dioxane, the resulting residue was taken up by water and lyophilized to give TCEP-18 (152.7 mg, 88.2%) as white solid. MS[M−H]⁻=253.19, exact mass calc. for C₁₂H₁₅O₄P is 254.07. ¹H NMR (400 MHz, DMSO-d₆): δ7.74 (dd, J=10.9, 7.3 Hz, 2H), 7.62-7.49 (m, 3H), 2.46-2.35 (m, 2H), 2.33-2.00 (m, 6H).

### Example 17: Synthesis of TCEP-19

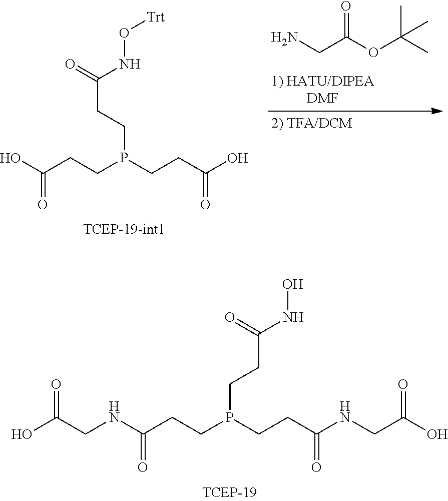

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N2 atmosphere. The mixture was stirred for 30 min, tert-Butyl glycinate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-19 (10.2 mg, 6.8%) as brown solid. MS[M+H]⁺=380.24, exact mass calc. for C₁₃H₂₂N₃O₈P is 379.31. ¹H-NMR (400 MHz, Deuterium Oxide): b 3.99 (s, 4H), 2.93-2.84 (m, 4H), 2.76-2.53 (m, 8H).

### Example 18: Synthesis of TCEP-20

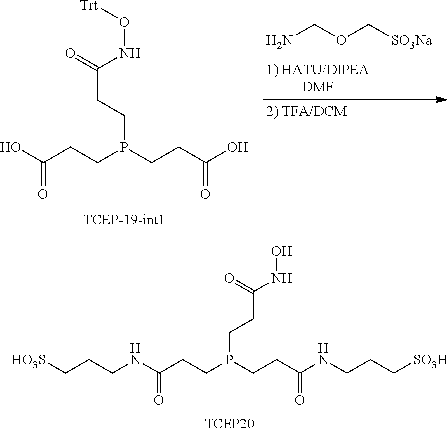

To a solution of TCEP-19-int1 (200 mg, 0.39 mmol, 1.0 eq.) in DMF (3 mL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Sodium 3-Aminopropane-1-Sulfonate (1 mmol, Adamas) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (23.7 mg, 11.7%) as brown solid. MS[M−H]⁻=506.22, exact mass calc. for C₁₅H₃₀N₃O₁₀PS₂ is 507.51. ¹H NMR (400 MHz, Deuterium Oxide): δ 3.27 (t, J=6.8 Hz, 4H), 2.91-2.85 (m, 4H), 2.80-2.71 (m, 4H), 2.69-2.61 (m, 2H), 2.58-2.49 (m, 6H), 1.94-1.85 (m, 4H).

### Example 19: Synthesis of TCEP-21

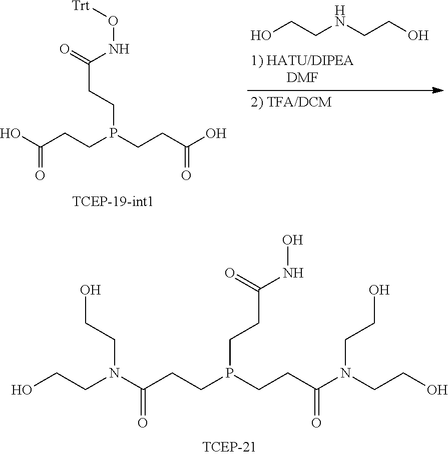

To a solution of TCEP-19-int1 (200 mg, 0.39 mmol, 1.0 eq.) in DMF (3 nL) was added HATU (380 mg, 1.0 mmol, 2.5 eq) followed by DIPEA (174 μL, 1.0 mmol, 2.5 eq) at 0° C. under N₂ atmosphere. The mixture was stirred for 30 min, Diethanolamine (1 mmol, Bidepharm) was added. The reaction was stirred at room temperature for 1 h. The reaction mixture was purified by RP-HPLC using a C18 column yielding the protected product. The product was dissolved in DCM (3 mL) and TFA (0.5 mL) was added. The mixture was stirred for 1 h, LCMS showed reaction was completed. The mixture was concentrated and the residue was taken up by distilled water (10 mL), washed with EtOAc (2*5 mL). The aqueous layer was lyophilized to give TCEP-20 (13.8 mg, 7.8 yield) as white solid. MS[M+H]⁻=440.27, exact mass calc. for C₁₇H₃₄N₃O₈P is 439.45. ¹H NMR (400 MHz, Deuterium Oxide): δ 4.50-4.28 (m, 4H), 3.78 (t, J=5.2 Hz, 4H), 3.38 (t, J=5.1 Hz, 4H), 3.17 (q, J=5.2 Hz, 4H), 2.96-2.80 (m, 4H), 2.68-2.51 (m, 8H).

### Example 20: Synthesis of TCEP-23

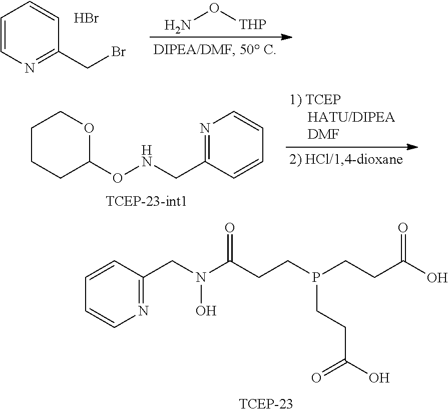

1. TCEP-23-int1

To a solution of 2-(Aminooxy)tetrahydro-2H-pyran (1.17 g, 10 mmol, 2.0 eq, Bidepharm) in DMF (15 mL) was added DIPEA (3.5 mL, 20 mmol, 4.eq) followed by 2-(Bromomethyl)pyridine hydrobromide (1.3 g, 5.0 mmol, 1.0 eq, Adamas). The mixture was stirred for 16 h at 50° C. The reaction mixture was poured into water (100 mL), extracted with EtOAc (30M1*3). The organic layer was washed with brine (30 mL), dried over Na₂SO₄ and filtered. The filtrate was concentrated and purified by flash column, to give TCEP-23-int1 (800 mg, 80%), as colorless oil.

### 2. TCEP-23

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-23-int1 (100 mg, 0.5 mmol, 1.0 eq) was added. The reaction was stirred at room temperature for 4 h. The reaction mixture was purified by RP-HPLC using a C18 column yielding the desired product. The product was dissolved in HCl/1,4-dioxane (3 mL). The mixture was stirred for 1 h, LCMS showed reaction was completed. The mixture was concentrated and the residue was taken up by distilled water, lyophilized to give TCEP-23 (17.9 mg, 10.0%) as white solid. MS[M+H]⁺=357.19, exact mass calc. for C₁₅H₂₁N₂O₆P is 356.31. ¹H-NMR (400 MHz, Deuterium Oxide): δ 8.73 (dd, J=6.3, 1.7 Hz, 1H), 8.59 (td, J=7.9, 1.6 Hz, 1H), 8.02 (dd, J=6.4, 3.4 Hz, 2H), 5.21 (s, 2H), 3.21-2.97 (m, 2H), 2.95-2.80 (m, 4H), 2.69-2.56 (m, 6H).

### Example 21: Synthesis of TCEP-24

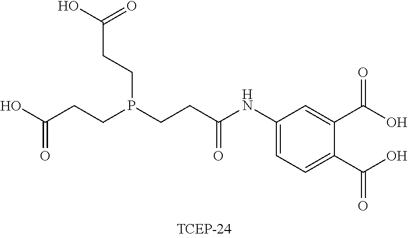 [SMILES: O=C(O)CCP(CCC(=O)O)CCC(=O)Nc1ccc(C(=O)O)c(C(=O)O)c1]

TCEP-24 was synthesized as the synthesis procedure A wherein (4-Aminophthalic acid, Bidepharm) was amine reagent, yielding TCEP-24 (21.5 mg, 10.4% yield) as white solid. MS[M−H]⁻=412.22, exact mass calc. for C₁₇H₂₀NO₉P is 413.09. ¹H NMR (400 MHz, Deuterium Oxide): δ 7.87 (d, J=8.5 Hz, 1H), 7.77 (d, J=2.1 Hz, 1H), 7.71-7.64 (m, 1H), 3.08-3.00 (m, 2H), 2.96-2.86 (m, 4H), 2.70-2.59 (m, 6H).

### Example 22: Synthesis of TCEP-25

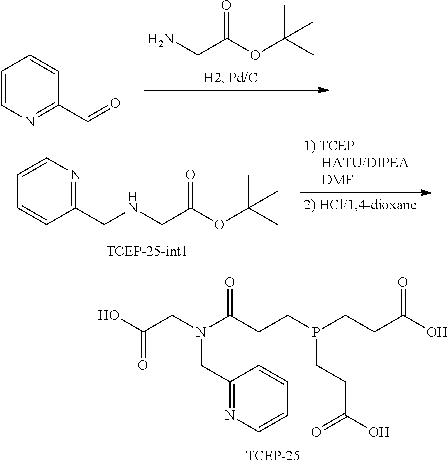

1. TCEP-25-int1

To a solution of 2-Pyridinecarboxaldehyde (1.0 g, 10 mmol, 1.0 eq. Adamas) and tert-Butyl glycinate (1.3 g, 10.0 mmol, 1.0 eq) in MeOH (25 mL) was added Pd/C (150 mg) and two drops of AcOH. The mixture was degassed 3 times and purged with H₂, then stirred for 16 h at room temperature under H₂ atmosphere. The reaction mixture was filtered through a Celite pad and the filtrate was concentrate then purified by flash column, to give TCEP-25-int1 (1.6 g, 72.0%) as yellow oil.

### 2. TCEP-25

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-25-int1 (l1 mg, 0.5 mmol, 1.0 eq) was added. The reaction was stirred at room temperature for 4 h. The reaction mixture was purified by RP-HPLC using a C18 column yielding the desired product. The product was dissolved in HCl/1,4-dioxane (3 mL). The mixture was stirred for 1 h, LCMS showed reaction was completed. The mixture was concentrated and the residue was taken up by distilled water, lyophilized to give TCEP-25 (51.3 mg, 17.2% yield) as white solid. MS[M+H]⁺=399.25, exact mass calc. for C₁₇H₂₃N₂O₇P is 398.12. ¹H NMR (400 MHz, Deuterium Oxide): δ 8.60 (dd, J=5.9, 1.6 Hz, 1H), 8.45 (td, J=8.0, 1.6 Hz, 1H), 7.92 (d, J=8.3 Hz, 1H), 7.87 (ddd, J=7.5, 5.9, 1.3 Hz, 1H), 4.88 (s, 2H), 4.39 (s, 2H), 2.84-2.68 (m, 6H), 2.51-2.41 (m, 6H).

### Example 23: Synthesis of TCEP-26

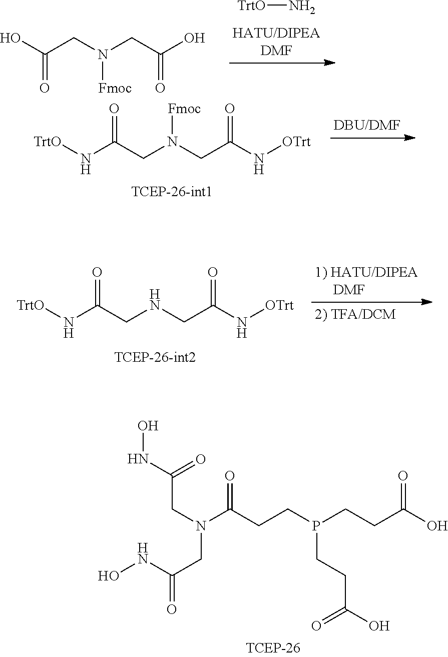

1. TCEP-26-int1

To a solution of Fmoc-iminodiacetic acid (1.8 g, 5.0 mmol, 1.0 eq, Bidepharm) in DMF (30 mL) was added HATU (4.3 g, 11.0 mmol, 2.2 eq) followed by DIPEA (2.0 mmol, 4.0 eq) under N₂ atmosphere. The mixture was stirred for 30 min and O-Tritylhydroxylamine (3.0 g, 11.0 mmol, 2.2 eq) was added. The reaction was stirred at room temperature for 4 h. The reaction mixture was poured into water (200 mL). The precipitate was collected by filtration and the filter cake was dried over vacuum to give TCEP-23-int1 (4.0 g, 92.0%), as white solid.

2. TCEP-26-int2

To a solution of TCEP-26-int1 (2.0 g, 2.3 mmol, 1.0 eq) in DMF (10 mL) was added DBU (2 mL). The mixture was stirred for 1 h at room temperature, then poured into ice-water (100 mL), extracted with EtOAc (50 mL*3). The combined organic layer was washed with brine, dried over Na₂SO₄ and filtered. The filtrate was concentrated and purified by flash column (EtOAc/petroleum ether=0˜50%, v/v) to give TCEP-26-int2 (1.2 g, 80%).

### 3. TCEP-26

The compound was synthesized as the synthesis procedure A-1 wherein TCEP-26-int2 was amine reagent, yielding TCEP-26 (31.5 mg, 21.3% yield) as white solid. MS[M+H]⁺=396.17, exact mass calc. for C₁₃H₂₂N₃O₉P is 395.11. ¹H NMR (400 MHz, Deuterium Oxide) δ 4.13 (s, 2H), 3.97 (s. 2H), 2.85-2.77 (m, 4H), 2.54-2.47 (m, 6H), 2.18-2.08 (m, 2H).

### Example 24: Synthesis of TCEP-28

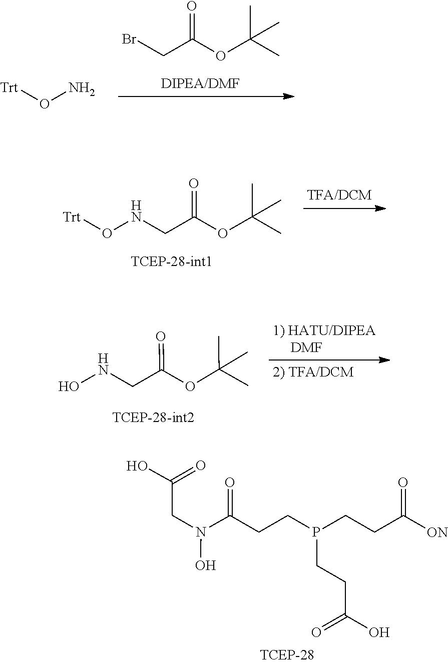

1. TCEP-28-int1

To a solution of O-Tritylhydroxylamine (1.4 g, 5.0 mmol, 1.0 eq) in DMF (15 mL) was added DIPEA (1.7 mL, 10 mmol, 2.eq) followed by tert-Butyl bromoacetate (1.0 g, 5.0 mmol, 1.0 eq, Adamas). The mixture was stirred for 16 h at 50° C. The reaction mixture was poured into water (100 mL), extracted with EtOAc (30 mL*3). The organic layer was washed with brine (30 mL), dried over Na₂SO₄ and filtered. The filtrate was concentrated and purified by flash column, to give TCEP-28-int1 (1.4 g, 70%), as white solid.

2. TCEP-28-int2

To a solution of TCEP-28-int1 (1.4 g, 3.6 mmol, 1.0 cq) in DCM (15 mL) was added TFA (1.5 mL). The mixture was stirred for 2 h at room temperature. The reaction mixture was concentrated and purified by flash column, to give TCEP-28-int2 (380 mg, 71.8%), as colorless oil.

### 3. TCEP-28

To a solution of TCEP (286.6 mg, 1.0 mmol, 2.0 eq.) in DMF (3 mL) was added HATU (190 mg, 0.5 mmol, 1 eq) followed by DIPEA (2.0 mmol, 4.0 eq) under N₂ atmosphere. The mixture was stirred for 30 min and TCEP-28-int2 (73.5 mg, 0.5 mmol, 1.0 eq) was added. The reaction was stirred at room temperature for 2 h. The reaction mixture was purified by RP-HPLC using a C18 column yielding the desired product. The product was dissolved in HCl/1,4-dioxane (3 mL, Adamas). The mixture was stirred for 1 h, LCMS showed reaction was completed. The mixture was concentrated and the residue was taken up by distilled water, lyophilized to give TCEP-28 (43.6 mg, 27.1% yield) as white solid. MS[M−H]=322.16, exact mass calc. for C₁₁H₁₈NO₈P is 323.08. ¹H NMR (400 MHz, Deuterium Oxide): δ 4.36 (s, 2H), 2.86-2.77 (m, 6H), 2.56-2.48 (m, 6H).

### Example 25: Synthesis of TCEP-30

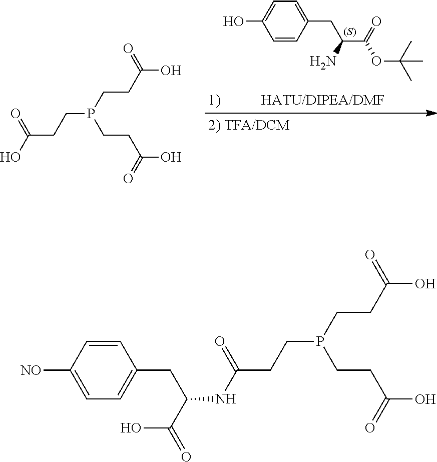

TCEP-30 was synthesized as the synthesis procedure A-1 wherein [tert-Butyl L-tyrosinate, Adamas) was amine reagent, yielding TCEP-30 (25.3 mg, 12.25%) as white solid. MS[M+H]⁺=414.23, exact mass calc. for C₁₈H₂₄NO₈P is 413.12. ¹H NMR (400 MHz, Deuterium Oxide): δ 7.24-7.14 (m, 2H), 6.91-6.82 (m, 2H), 4.67 (dd, J=10.3, 4.7 Hz, 1H), 3.26 (dd, J=14.0, 4.7 Hz, 1H), 2.92-2.64 (m, 7H), 2.57-2.30 (m, 6H).

### Example 26: Synthesis of TCEP-31

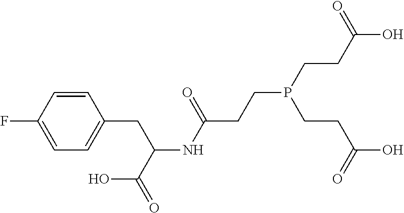 [SMILES: O=C(O)CCP(CCC(=O)O)CCC(=O)NC(Cc1ccc(F)cc1)C(=O)O]

TCEP-31 was synthesized as the synthesis procedure A wherein (DL-3-(4-Fluorophenyl)alanine, Bidepharm) was amine reagent, yielding TCEP-31 (27.1 mg, 13.10%) as white solid. MS[M+H]⁺=416.01, exact mass calc. for C₁₈H₂₃NO₇P is 415.12. ¹H NMR (400 MHz, Deuterium Oxide): δ 7.36-7.26 (m, 2H), 7.12 (t, J=8.8 Hz, 2H), 4.70 (dd, J=10.1, 4.8 Hz, 1H), 3.31 (dd, J=14.0, 4.9 Hz, 1H), 2.95 (dd, J=14.0, 10.1 Hz, 1H), 2.90-2.65 (m, 6H), 2.56-2.40 (m, 6H).

### Example 27: Synthesis of TCEP-32

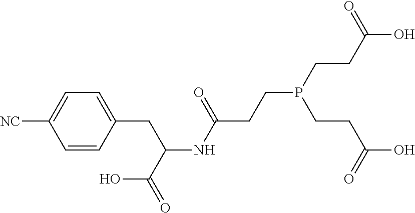 [SMILES: N#Cc1ccc(CC(NC(=O)CCP(CCC(=O)O)CCC(=O)O)C(=O)O)cc1]

TCEP-32 was synthesized as the synthesis procedure A wherein (DL-4-Cyanophenylalanine, Bidepharm) was amine reagent, yielding TCEP-32 (18.5 mg, 8.76%) as white solid. MS[M+H]⁺=423.24, exact mass calc. for C₁₉H₂₃N207P is 422.12. ¹H NMR (400 MHz, Deuterium Oxide): δ 7.77 (d, J=8.1 Hz, 2H), 7.49 (d, J=8.1 Hz, 2H), 4.77-4.71 (m, 1H), 3.42 (dd, J=14.0, 5.0 Hz, 1H), 3.05 (dd, J=14.0, 10.0 Hz, 1H), 2.91-2.70 (m, 6H), 2.68-2.39 (m, 6H).

### Example 28: Synthesis of TCEP-33

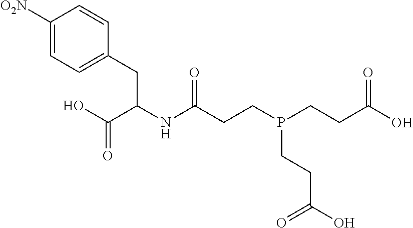 [SMILES: O=C(O)CCP(CCC(=O)O)CCC(=O)NC(Cc1ccc([N+](=O)[O-])cc1)C(=O)O]

TCEP-33 was synthesized as the synthesis procedure A wherein (DL-4-nitro-phenylalanine, Bidepharm) was amine reagent, yielding TCEP-33 (20.7 mg, 9.37%) as white solid. MS[M+H]⁺=443.24, exact mass calc. for C₁₉H₂₃N₂O₉P is 442.11. ¹H NMR (400 MHz, Deuterium Oxide): δ 8.22 (d, J=8.2 Hz, 2H), 7.54 (d, J=8.2 Hz, 2H), 4.84-4.80 (m, 1H), 3.47 (dd, J=14.0, 4.8 Hz, 1H), 3.11 (dd, J=13.9, 10.3 Hz, 1H), 2.91-2.73 (m, 6H), 2.58-2.38 (m, 6H).

### Example 29: Synthesis of TCEP-34

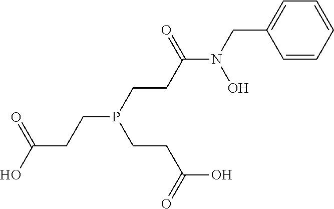 [SMILES: O=C(O)CCP(CCC(=O)O)CCC(=O)N(O)Cc1ccccc1]

TCEP-34 was synthesized as the synthesis procedure A wherein (N-Benzylhydroxylamine hydrochloride, Bidepharm) was amine reagent, yielding TCEP-34 (15.7 mg, 8.85%) as white solid. MS[M+H]⁺=356.05, exact mass calc. for C₁₆H₂₂NO₆P is 355.12. ¹H NMR (400 MHz, Deuterium Oxide): δ 7.55-7.34 (m, 5H), 4.84 (s, 2H), 3.14 (dt, J=19.8, 6.9 Hz, 2H), 2.92 (dt, J=18.2, 7.0 Hz, 4H), 2.62 (dq, J=14.5, 7.3 Hz, 6H).

### Example 30: Synthesis of TCEP-35

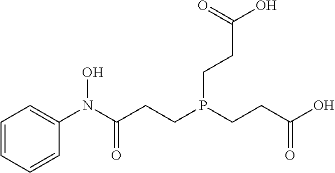 [SMILES: O=C(O)CCP(CCC(=O)O)CCC(=O)N(O)c1ccccc1]

TCEP-35 was synthesized as the synthesis procedure A wherein (N-Phenylhydroxylamine, Bidepharm) was amine reagent, yielding TCEP-35 (17.1 mg, 10.0%) as white solid. MS[M+H]⁺=341.97, exact mass calc. for C₁₅H₂NO₆P is 341.10. ¹H NMR (400 MHz, Deuterium Oxide): δ 7.57-6.88 (m, 5H), 3.27-3.22 (m, 1H), 2.92-2.78 (m, 3H), 2.65-2.53 (m, 6H), 2.29-1.98 (m, 2H).

### Example 31: Synthesis of TCEP-37

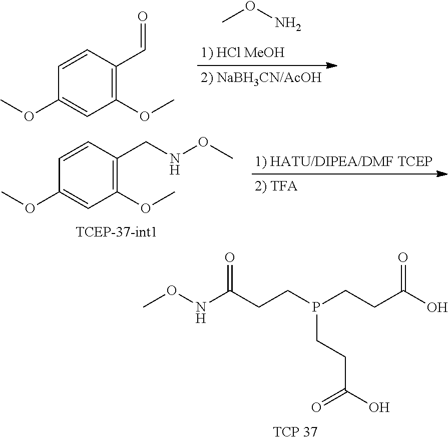

1. TCEP-37-int1

To a solution of 2,4-Dimethoxybenzaldehyde (1.66 g, 10.0 mmol, 1.0 eq, Adamas) in MeOH (25 mL) was added O-methylhydroxylamine hydrochloride (1.66 g, 20.0 mmol, 2.0 eq, Bidepharm). The resulting mixture was stirred for 16 h at room temperature. LCMS showed reaction was completed. The mixture was concentrated and the residue was taken up by AcOH (20 mL), NaBH₃CN was added and stirred for 5 h at room temperature. LCMS showed reaction was completed. The reaction mixture was concentrated and residue was poured into ice-water (200 mL) extracted with EtOAc (50 mL*3). The combined organic layer was dried over Na₂SO₄ and filtered. The filtrate was concentrated over vacuum and purified with flash column (EtOAc/petroleum ether=0-50%) to give product TCEP-37-int1 (N-(2,4-dimethoxybenzyl)-O-methyl hydroxylamine, 1.5 g, 76.1%, colorless oil). 2. TCEP-37 TCEP-37 was synthesized as the synthesis procedure A-1 wherein TCEP-73-int1 was amine reagent, yielding TCEP-37 (12.8 mg, 9.14%) as white solid. MS[M+H]⁺=280.18, exact mass calc. for C₁₀H₁₈NO₆P is 279.09. ¹H NMR (400 MHz, Deuterium Oxide): δ (s, 3H), 2.89 (dt, J=18.4, 7.1 Hz, 4H), 2.72 (dd, J=18.1, 6.6 Hz, 2H), 2.61 (dq, J=13.9, 6.7 Hz, 6H).

### Example 32: Preparation of Trastuzumab-[MC-VC-PAB-MMAE]₆ Conjugate (The ADC with D6)

The ADC was prepared in a one-pot reaction:
(1) ZnCl₂ (0.024 mM) and reductant TCEP-NO-Trtyl prepared by Example 1 (0.048 mM) were added to a solution of a monoclonal antibody Trastuzumab (0.012 mM, in BES buffer, pH7.0, 20 mM) and the reaction mixture was allowed to stay at 4° C. for 18 h;
(2) EDTA-2Na (0.6 mM) and MC-VC-PAB-MMAE (0.096 mM) in DMA was introduced and the reaction was continued at room temperature for 1 h;
(3) The reaction mixture was subjected to purification using a desalting column.

### Example 33: Preparation of Trastuzumab-[MC-VC-PAB-MMAE]₆ Conjugate (The ADC with D6)

The method of Example 33 is similar to Example 32, and the difference was that the reductant was TCEP-NO prepared by Example 1.

### Examples 34-57 and the Comparative Example 5: Preparation of ADCs with D6

The method of examples 34-57 was similar to example 32, and the differences were the kinds of reductant and the antibody, the molar ratio of the reductant and the antibody, and/or the molar ratio of the molar ratio of the Zn²⁺ and the reductant which are shown in as follow table. Meanwhile, and the incubation time in step (1) is 16 h in examples 55-57.

| | | Reductant | Reductant/mAb | ZnCl₂ | ZnCl₂/Reductant | Antibody |
|---|---|---|---|---|---|---|
| No. | Reductant | (mM) | (Molar Ratio) | (mM) | (Molar Ratio) | (mAb) |
| E34 | TCEP-1 | 0.054 | 4.5:1 | 0.024 | 0.44:1 | Trastuzumab |
| E35 | TCEP-2 | 0.144 | 12:1 | 0.024 | 0.17:1 | Trastuzumab |
| E36 | TCEP-3 | 0.120 | 10:1 | 0.024 | 0.20:1 | Trastuzumab |
| E37 | TCEP-4 | 0.120 | 10:1 | 0.024 | 0.20:1 | Trastuzumab |
| E38 | TCEP-5 | 0.144 | 12:1 | 0.024 | 0.25:1 | Trastuzumab |
| E39 | TCEP-6 | 0.103 | 8.6:1 | 0.024 | 0.23:1 | Trastuzumab |
| E40 | TCEP-7 | 0.036 | 3:1 | 0.012 | 0.34:1 | Trastuzumab |
| E41 | TCEP-8 | 0.048 | 4:1 | 0.024 | 0.50:1 | Trastuzumab |
| E42 | TCEP-9 | 0.048 | 4:1 | 0.024 | 0.50:1 | Trastuzumab |
| E43 | TCEP-10 | 0.048 | 4:1 | 0.024 | 0.50:1 | Trastuzumab |
| E44 | TCEP-15 | 0.048 | 4:1 | 0.024 | 0.50:1 | Trastuzumab |
| E45 | TCEP-18 | 0.054 | 4.5:1 | 0.024 | 0.44:1 | Trastuzumab |
| E46 | TCEP-19 | 0.077 | 6.4:1 | 0.024 | 0.31:1 | Trastuzumab |
| E47 | TCEP-20 | 0.103 | 8.6:1 | 0.024 | 0.23:1 | Trastuzumab |
| E48 | TCEP-23 | 0.077 | 6.4:1 | 0.024 | 0.31:1 | Trastuzumab |
| F49 | TCEP-24 | 0.046 | 3.8:1 | 0.024 | 0.53:1 | Trastuzumab |
| E50 | TCEP-25 | 0.103 | 8.6:1 | 0.024 | 0.23:1 | Trastuzumab |
| E51 | TCEP-26 | 0.096 | 8:1 | 0.024 | 0.25:1 | Trastuzumab |
| E52 | TCEP-28 | 0.103 | 8.6:1 | 0.024 | 0.23:1 | Trastuzumab |
| E53 | TCEPA | 0.077 | 6.4:1 | 0.024 | 0.31:1 | Belantamab |
| E54 | TCEPA | 0.042 | 3.5:1 | 0.024 | 0.57:1 | Sacituzumab |
| E55 | TCEP-30 | 0.077 | 6.4:1 | 0.024 | 0.31:1 | Trastuzumab |
| E56 | TCEP-31 | 0.077 | 6.4:1 | 0.024 | 0.31:1 | Trastuzumab |
| E57 | TCEP-33 | 0.077 | 6.4:1 | 0.024 | 0.31:1 | Trastuzumab |
| C5 | TCEP | 0.048 | 4:1 | 0 | 0:1 | Trastuzumab |

“E” was short for Example, “C” was short for Comparative Example, and mAb is short for monoclonal antibody.

The homogeneity assays results of examples 32-57 and the comparative example 5 were shown as follows:

TABLE 1

| | | | D 0 | D 2 | D 4 | D 6 | D 8 |
|---|---|---|---|---|---|---|---|
| NO. | FIG. | Reductant | (%) | (%) | (%) | (%) | (%) |
| E32 | 1 | TCEP-NO-Trtyl | 0 | 11 | 9 | 66 | 14 |
| E33 | 2 | TCEP-NO | 0 | 4 | 16 | 70 | 10 |
| E34 | 3A | TCEP1 | 0 | 0 | 3.75 | 90.59 | 5.66 |
| E35 | 3B | TCEP-2 | 0 | 1.19 | 2.81 | 93.02 | 2.98 |
| E36 | 3C | TCEP-3 | 0 | 0 | 3.09 | 95.52 | 1.39 |
| E37 | 3D | TCEP-4 | 0 | 0 | 2.78 | 88.40 | 8.82 |
| E38 | 3E | TCEP-5 | 0 | 0 | 2.27 | 88.04 | 9.69 |
| E39 | 3F | TCEP-6 | 0 | 3.11 | 5.47 | 82.56 | 8.86 |
| E40 | 3G | TCEP-7 | 0 | 1.4 | 2.502 | 83.08 | 13.07 |
| E41 | 3H | TCEP-8 | 0 | 2.79 | 3.91 | 89.59 | 3.71 |
| E42 | 4A | TCEP-9 | 0 | 13.36 | 5.52 | 69.33 | 11.79 |
| E43 | 4B | TCEP-10 | 0 | 0 | 1.96 | 90.39 | 7.65 |
| E44 | 4C | TCEP-15 | 0 | 1.50 | 6.41 | 89.06 | 3.04 |
| E45 | 4D | TCEP-18 | 0 | 3.26 | 5.68 | 80.77 | 10.30 |
| E46 | 4E | TCEP-19 | 0 | 5.13 | 5.76 | 79.65 | 9.47 |
| E47 | 4F | TCEP-20 | 0 | 2.40 | 3.30 | 74.78 | 19.53 |
| E48 | 4G | TCEP-23 | 0 | 2.12 | 4.86 | 85.68 | 7.35 |
| E49 | 4H | TCEP-24 | 0 | 1.91 | 1.65 | 81.56 | 14.87 |
| E50 | 5A | TCEP-25 | 0 | 0.75 | 4.28 | 91.37 | 3.61 |
| E51 | 5B | TCEP-26 | 0 | 0 | 3.03 | 90.67 | 6.30 |
| E52 | 5C | TCEP-28 | 0 | 0 | 3.20 | 92.75 | 4.06 |
| E53 | 5D | TCEPA | 0 | 6.77 | 11.44 | 71.23 | 10.57 |
| E54 | 5E | TCEPA | 0 | 0 | 0 | 77.51 | 22.49 |
| E55 | 5F | TCEP-30 | 0 | 1.11 | 3.38 | 92.55 | 2.97 |
| E56 | 5G | TCEP-31 | 0 | 1.54 | 5.39 | 93.07 | 0 |
| E57 | 5H | TCEP-33 | 0 | 2.20 | 10.16 | 87.65 | 0 |
| C5 | 6B | TCEP | 0 | 0 | 9 | 10 | 81 |

As shown in the above table, linker-payloads (MC-VC-PAB-MMAE) were successfully linked to the antibody, which indicated ADCs of example 32-57 prepared by the reductant in the present application were successfully synthesized.

According to examples 32-57 and comparative example 5, the reductant in the present application could increase the homogeneity of the ADC with D6 compared with the traditional method using TCEP without Zn²⁺, which successfully demonstrated that combination of the transition metal ions and the novel reductants is responsible for higher level of D6 in the resultant ADCs. Further, the selective reductant ability of TCEP-3 is best, with a D6 content of up to 95.52%. Meanwhile, the selective reduction ability of TCEP-1, TCEP-2, TCEP-10, TCEP-25, TCEP-26, TCEP-28, TCEP-30, TCEP-31 and TCEP-33 is also wonderful, with a D6 content of up to 90%.

According to the results of examples 53 and 54, the reductants in the present application are suitable for preparing the ADC with different antibody.

### Examples 58-78: Preparation of Trastuzumab-[MC-VC-PAB-MMAE]₆ Conjugate

The preparation of examples 58-78 was similar to the preparation of ADC with D6 of Example 33, but it adjusted the reductant, the molar ratio of the reductant and the monoclonal antibody, the molar ratio of the ZnCl₂ and the monoclonal antibody, and/or the reduction time in step (a) which were shown as follows:

| | | | | | | Reductant |
|---|---|---|---|---|---|---|
| | | Reductant/mAb | ZnCl₂/mAb | ZnCl₂/Reductant | mAb | time in step |
| No. | Reductant | (Molar Ratio) | (Molar Ratio) | (Molar Ratio) | (mM) | (1) (h) |
| E58 | TCEPA | 2.8:1 | 2:1 | 0.71:1 | 0.012 | 18 |
| E59 | TCEPA | 3.5:1 | 2:1 | 0.57:1 | 0.012 | 18 |
| E60 | TCEPA | 3.8:1 | 2:1 | 0.52:1 | 0.012 | 18 |
| E61 | TCEPA | 4:1 | 2:1 | 0.5:1 | 0.012 | 18 |
| E62 | TCEPA | 4.2:1 | 2:1 | 0.47:1 | 0.012 | 18 |
| E63 | TCEPA | 4.4:1 | 2:1 | 0.45:1 | 0.012 | 18 |
| E64 | TCEPA | 4.6:1 | 2:1 | 0.43:1 | 0.012 | 18 |
| E65 | TCEPA | 3.8:1 | 1:1 | 0.26:1 | 0.012 | 18 |
| E66 | TCEPA | 4:1 | 1:1 | 0.25:1 | 0.012 | 18 |
| E67 | TCEPA | 4.2:1 | 1:1 | 0.23:1 | 0.012 | 18 |
| E68 | TCEPA | 4.4:1 | 1:1 | 0.22:1 | 0.012 | 18 |
| E69 | TCEPA | 4.6:1 | 1:1 | 0.21:1 | 0.012 | 18 |
| E70 | TCEP-NO | 5:1 | 1:1 | 0.20:1 | 0.012 | 6 |
| E71 | TCEP-NO | 6:1 | 1:1 | 0.16:1 | 0.012 | 6 |
| E72 | TCEP-NO | 7:1 | 1:1 | 0.14:1 | 0.012 | 6 |
| E73 | TCEP-NO | 8:1 | 1:1 | 0.125:1 | 0.012 | 6 |
| E74 | TCEP-NO | 9:1 | 1:1 | 0.11:1 | 0.012 | 6 |
| E75 | TCEP-NO | 10:1 | 1:1 | 0.1:1 | 0.012 | 6 |
| E76 | TCEP-NO | 11:1 | 1:1 | 0.09:1 | 0.012 | 6 |
| E77 | TCEP-NO | 12:1 | 1:1 | 0.083:1 | 0.012 | 6 |
| E78 | TCEP-NO | 13:1 | 1:1 | 0.077:1 | 0.012 | 6 |

The homogeneity assays results were shown as follows:

TABLE 2

| | | | Reductant | | | | | |
|---|---|---|---|---|---|---|---|---|
| | | Reductant/mAb | time in step | D 0 | D 2 | D 4 | D 6 | D 8 |
| Example | FIG. | (Molar Ratio) | (1) (h) | (%) | (%) | (%) | (%) | (%) |
| E58 | 7A | 2.8:1 | 18 | 0 | 11.11 | 13.35 | 75.54 | 0 |
| E59 | 7B | 3.5:1 | 18 | 0 | 5.35 | 8.07 | 84.71 | 1.87 |
| E60 | 8 | 3.8:1 | 18 | 0 | 5 | 10 | 81 | 4 |
| E61 | 9 | 4:1 | 18 | 0 | 4 | 10 | 81 | 5 |
| E62 | 10 | 4.2:1 | 18 | 0 | 3 | 6 | 83 | 8 |
| E63 | 11 | 4.4:1 | 18 | 0 | 3 | 6 | 83 | 7 |
| E64 | 12 | 4.6:1 | 18 | 0 | 3 | 7 | 82 | 8 |
| E65 | 13 | 3.8:1 | 18 | 0 | 5 | 24 | 70 | 1 |
| E66 | 14 | 4:1 | 18 | 0 | 4 | 17 | 76 | 3 |
| E67 | 15 | 4.2:1 | 18 | 0 | 3 | 15 | 78 | 4 |
| E68 | 16 | 4.4:1 | 18 | 0 | 4 | 17 | 75 | 4 |
| E69 | 17 | 4.6:1 | 18 | 0 | 3 | 12 | 79 | 6 |
| E70 | 18A | 5:1 | 6 | 0 | 23.69 | 32.30 | 44.01 | 0 |
| E71 | 18B | 6:1 | 6 | 0 | 13.74 | 21.65 | 64.60 | 0 |
| E72 | 18C | 7:1 | 6 | 0 | 11.16 | 22.20 | 66.64 | 0 |
| E73 | 19A | 8:1 | 6 | 0 | 4.69 | 5.88 | 81.04 | 8.40 |
| E74 | 19B | 9:1 | 6 | 0 | 3.81 | 5.02 | 82.00 | 9.17 |
| E75 | 19C | 10:1 | 6 | 0 | 2.79 | 4.07 | 83.81 | 9.33 |
| E76 | 19D | 11:1 | 6 | 0 | 1.14 | 2.74 | 82.19 | 13.93 |
| E77 | 19E | 12:1 | 6 | 0 | 1.25 | 2.70 | 82.22 | 13.84 |
| E78 | 19F | 13:1 | 6 | 0 | 1.95 | 2.79 | 81.04 | 14.23 |

The results of Examples 58-78 were shown in Table 2. As the results shown in table 2, linker-payloads (MC-VC-PAB-MMAE) were successfully linked to Trastuzumab, which indicated ADCs of Examples 58-78 prepared by TCEPA or TCEP-NO were successfully synthesized. TCEPA and TCEP-NO could be used as a reductant in antibody modification and preparation of ADC with D6.

According to the results of table 2, the highest proportion of D6 was 84.71%, when the molar ratio of the ZnCl₂ and the monoclonal antibody was 2 and the molar ratio of the reductant and the antibody was 2.8:1 to 4.6:1. And D6 was at least 40% when the molar ratio of the ZnCl₂ and the monoclonal antibody was 1 and the molar ratio of the reductant and the antibody was 3.8:1 to 13:1. The resultant ADCs with high level of D6 showed that, the molar ratio of the reductant and the antibody ranging from 2.5 to 13 was benefit for improving the homogeneity of ADCs.

According to examples 70-78, when the molar ratio of the reductant and the antibody is from 5:1 to 13:1, the reduction time in step (1) is shortened to 6 h. When the molar ratio of the reductant and the antibody is from 6:1 to 13:1 and the reduction time in step (1) is 6 h, the content of D6 is up to 65%, 70%, even to 75% or 80%.

### Examples 79-90: Preparation of Trastuzumab-[MC-VC-PAB-MMAE]₆ Conjugate with Different Molar Ratio of the ZnCl₂ and the Reductant

The preparation of Trastuzumab-[MC-VC-PAB-MMAE]₆ conjugate was similar to the preparation of ADC of Example 32, but the reductant was TCEPA or TCEP-NO.

The reductant, the molar ratio of the ZnCl₂ and the reductant and the reductant time in step (1) were shown in follow table.

### Comparative Examples 1-4: Preparation of Trastuzumab-[MC-VC-PAB-MMAE]₆ Conjugate (the Concentration of Zn²⁺ is 0)

The preparation of comparative example 1 was similar to the preparation of ADC of Example 32, but the reductant was TCEPA. The preparation of comparative example 2 was similar to that of Example 73, the preparation of comparative example 3 was similar to that of Example 75, the preparation of comparative example 4 was similar to that of Example 77, the difference is that the concentration of ZnCl₂ in step (1) is 0.

| | | ZnCl₂/ | | Reductant/ | The reductant |
|---|---|---|---|---|---|
| | | Reductant | | mAb | time in |
| | | (Molar | mAb | (Molar | step (1) |
| Example | Reductant | Ratio) | (mM) | Ratio) | (h) |
| C1 | TCEPA | 0 | 0.012 | 4:1 | 18 |
| C2 | TCEP-NO | 0 | 0.012 | 8:1 | 6 |
| C3 | TCEP-NO | 0 | 0.012 | 10:1 | 6 |
| C4 | TCEP-NO | 0 | 0.012 | 12:1 | 6 |
| E79 | TCEPA | 0.25:1 | 0.012 | 4 | 18 |
| E80 | TCEPA | 0.5:1 | 0.012 | 4 | 18 |
| E81 | TCEPA | 1:1 | 0.012 | 4 | 18 |
| E82 | TCEPA | 2:1 | 0.012 | 4 | 18 |
| E83 | TCEPA | 3:1 | 0.012 | 4 | 18 |
| E84 | TCEPA | 4:1 | 0.012 | 4 | 18 |
| E85 | TCEPA | 7:1 | 0.012 | 4 | 18 |
| E86 | TCEPA | 7.5:1 | 0.012 | 4 | 18 |
| E87 | TCEPA | 15:1 | 0.012 | 3.2:1 | 18 |
| E88 | TCEPA | 30:1 | 0.012 | 3.2:1 | 18 |
| E89 | TCEP-NO | 0.22:1 | 0.012 | 9:1 | 4 |
| E90 | TCEP-NO | 3.33:1 | 0.012 | 9:1 | 4 |

The homogeneity assays results of examples 79-90 and comparative examples 1-4 were shown as follows:

TABLE 3

| | | ZnCl₂/Reductant | D 0 | D 2 | D 4 | D 6 | D 8 |
|---|---|---|---|---|---|---|---|
| Example | FIG. | (Molar Ratio) | (wt %) | (wt %) | (wt %) | (wt %) | (wt %) |
| C1 | 20 | 0 | 0 | 2 | 18 | 20 | 60 |
| C2 | 21A | 0 | 0 | 0 | 24.55 | 30.92 | 44.54 |
| C3 | 21B | 0 | 0 | 0 | 6.97 | 17.14 | 75.89 |
| C4 | 21C | 0 | 0 | 0 | 0 | 11.56 | 88.44 |
| E79 | 22 | 0.25:1 | 0 | 3 | 12 | 79 | 6 |
| E80 | 23 | 0.5:1 | 0 | 3 | 6 | 83 | 8 |
| E81 | 24 | 1:1 | 0 | 3 | 5 | 82 | 10 |
| E82 | 25 | 2:1 | 0 | 3 | 6 | 83 | 8 |
| E83 | 26 | 3:1 | 0 | 0 | 5 | 83 | 12 |
| E84 | 27 | 4:1 | 0 | 0 | 6 | 81 | 13 |
| E85 | 28 | 7:1 | 0 | 0 | 5 | 79 | 16 |
| E86 | 29A | 7.5:1 | 0 | 0 | 5.29 | 79.31 | 15.40 |
| E87 | 29B | 15:1 | 0 | 2.94 | 6.64 | 85.07 | 5.36 |
| E88 | 29C | 30:1 | 0 | 2.08 | 7.43 | 78.90 | 11.59 |
| E89 | 29D | 0.22:1 | 0 | 4.12 | 5.79 | 77.34 | 12.75 |
| E90 | 29E | 3.33:1 | 4.81 | 2.83 | 4.49 | 63.04 | 24.83 |

As the results shown in table 3, linker-payloads (MC-VC-PAB-MMAE) were successfully linked to Trastuzumab, which indicated ADCs prepared with TCEPA/TCEP-NO and Zn²⁺ of example 79-90 were successfully synthesized.

According to the results of Table 3, The proportion of D6 in comparative examples 1-4 was only 11%, 17%, 20% or 31%, but the proportion of D6 in Examples 79-90 was at least 60%. It showed that Zn²⁺ was important to improve the homogeneity of ADCs with D6. Further, the molar ratio of Zn²⁺ and the reductant ranging from 0.05 to 30 was benefit for improving the homogeneity of ADCs.

### Examples 91-102: Preparationof Trastuzumab-[MC-VC-PAB-MMAE]₆ Conjugate in Different Buffers

The preparation of Trastuzumab-[MC-VC-PAB-MMAE]₆ conjugate was similar to the preparation of ADC of Example 82, the molar ratio of the TCEPA and the antibody, and the buffer system was as follows:

| | | TCEPA/mAb | ZnCl₂/mAb | |
|---|---|---|---|---|
| Example | The buffer system | (Molar Ratio) | (Molar Ratio) | pH |
| E91 | MOBS buffer | 4:1 | 2:1 | 7.4 |
| E92 | TAPSO buffer | 4:1 | 2:1 | 7.4 |
| E93 | DIPSO buffer | 4:1 | 2:1 | 7.4 |
| E94 | MOPSO buffer | 4:1 | 2:1 | 7.4 |
| E95 | ACES buffer | 4:1 | 2:1 | 7.4 |
| E96 | Bis-Tris buffer | 3.5:1 | 2:1 | 6.7 |
| E97 | PIPES buffer | 3.5:1 | 2:1 | 6.7 |
| E98 | HEPES buffer | 3.5:1 | 2:1 | 7.0 |
| E99 | MOPS buffer | 3.5:1 | 2:1 | 7.0 |
| E100 | TES buffer | 3.5:1 | 2:1 | 7.4 |
| E101 | PB | 3.5:1 | 2:1 | 6.7 |
| E102 | ADA buffer | 3.5:1 | 2:1 | 6.7 |

mAb is short for monoclonal antibody. All the buffers were commercially available from Macklin.

The homogeneity assays results were shown as follows:

TABLE 4

| | | D 0 | D 2 | D 4 | D 6 | D 8 |
|---|---|---|---|---|---|---|
| NO. | FIG. | (wt %) | (wt %) | (wt %) | (wt %) | (wt %) |
| E91 | 30 | 0 | 2 | 5 | 85 | 8 |
| E92 | 31 | 0 | 0 | 4 | 87 | 9 |
| E93 | 32 | 0 | 0 | 4 | 88 | 8 |
| E94 | 33 | 0 | 3 | 5 | 85 | 7 |
| E95 | 34 | 0 | 0 | 7 | 86 | 7 |
| E96 | 35A | 0.96 | 3.89 | 4.05 | 84.65 | 6.46 |
| E97 | 35B | 1.09 | 11.27 | 14.10 | 71.45 | 2.09 |
| E98 | 35C | 0 | 1.22 | 4.83 | 86.55 | 7.40 |
| E99 | 35D | 1.04 | 4.67 | 5.54 | 83.54 | 5.21 |
| E100 | 35E | 0 | 3.70 | 3.90 | 88.11 | 4.28 |
| E101 | 35F | 1.41 | 8.83 | 20.36 | 66.55 | 2.85 |
| E102 | 35G | 0 | 7.59 | 17.42 | 52.10 | 22.89 |

The results of examples 91-102 were shown in Table 4. As the results shown in table 4, linker-payloads (MC-VC-PAB-MMAE) were successfully linked to Trastuzumab, which indicated ADCs in different buffers were successfully synthesized.

According to the results of Table 4, The proportion of D6 was more than 50% in examples 91-102. Further, the proportion of D6 was more than 70% in examples 91-100, and the highest proportion of D6 was 88%. It showed that the buffer system in examples 91-102 were benefit for improving the homogeneity of ADCs with D6 and the proportion of D6.

### Examples 103-107: Preparation of Trastuzumab-[MC-VC-PAB-MMAE]₆ Conjugate with Different pH Value

The preparation of Trastuzumab-[MC-VC-PAB-MMAE]₆ conjugate was similar to the preparation of ADC of Example 82, but it adjusted the buffer pH value and the molar ratio of TCEPA and the antibody as follows:

| | The buffer | TCEPA/mAb | |
|---|---|---|---|
| Example | system | (Molar Ratio) | pH |
| E103 | BES buffer | 3.5:1 | 6.4 |
| E104 | BES buffer | 3.5:1 | 6.7 |
| E105 | BES buffer | 3.5:1 | 7.4 |
| E106 | MES buffer | 3.5:1 | 5.8 |
| E107 | MES buffer | 3.5:1 | 6.1 |

The homogeneity assays results were shown as follows:

TABLE 5

| | | D 0 | D 2 | D 4 | D 6 | D 8 |
|---|---|---|---|---|---|---|
| No. | FIG. | (wt %) | (wt %) | (wt %) | (wt %) | (wt %) |
| E103 | 36A | 0 | 6.21 | 8.05 | 80.67 | 5.07 |
| E104 | 36B | 0 | 5.34 | 13.90 | 77.02 | 3.75 |
| E105 | 36C | 0 | 2.73 | 14.61 | 79.28 | 3.38 |
| E106 | 36D | 1.43 | 14.67 | 25.41 | 58.49 | 0 |
| E107 | 36E | 0 | 13.45 | 24.28 | 60.53 | 1.74 |

The results of examples 103-107 were shown in Table 5. As the results shown in table 5, linker-payloads (MC-VC-PAB-MMAE) were successfully linked to Trastuzumab, which indicated ADCs with buffers pH value in a range from 5.8 to 6.7 were successfully synthesized.

According to the results of Table 5, The proportion of D6 was more than 58% in examples 103-107. Further, the proportion of D6 was more than 75% in examples 103-105, and the highest proportion of D6 was 80%.

### Examples 108-110: Preparation of Trastuzumab-[MC-VC-PAB-MMAE]₆ Conjugate with Different Concentrations of the Buffer System

The preparation of Trastuzumab-[MC-VC-PAB-MMAE]₆ conjugate was similar to the preparation of ADC of Example 82, but it adjusted the buffer concentration of the buffer system and the molar ratio of TCEPA and the antibody which were shown as follows:

| | The buffer | The concentration of the | TCEPA/mAb |
|---|---|---|---|
| Example | system | buffer system | (Molar Ratio) |
| E108 | BES buffer | 40 mM | 3.5:1 |
| E109 | BES buffer | 60 mM | 3.5:1 |
| E110 | BES buffer | 80 mM | 3.5:1 |

The homogeneity assays results were shown as follows:

TABLE 6

| | | D 0 | D 2 | D 4 | D 6 | D 8 |
|---|---|---|---|---|---|---|
| No. | FIG. | (wt %) | (wt %) | (wt %) | (wt %) | (wt %) |
| E108 | 37A | 0 | 5.83 | 6.32 | 84.06 | 3.79 |
| E109 | 37B | 1.70 | 5.14 | 3.72 | 85.50 | 3.95 |
| E110 | 37C | 0 | 7.10 | 6.95 | 83.64 | 2.31 |

As show in table 6, the results showed the concentration of the buffer systems of examples 108-110 are useful to increase the content of D6.

### Examples 111-119: Preparation of Trastuzumnab-[MC-VC-PAB-MMAE]₆ Conjugate (the Reduction Time and/or the Reductant Temperature in Step (1) is Different)

The method of examples 111-119 is the same as example 32, and the difference is the reduction time and/or the reductant temperature in step (1), the reductant, the molar ratio of the reductant and the antibody and/or the molar ratio of the ZnCl₂ and the antibody which are shown as follows,

| | The | The | | | |
|---|---|---|---|---|---|
| | reduction | reduction | | Reductant/ | ZnCl₂/ |
| | temperature | time in | | mAb | Reductant |
| | in step (1) | step (1) | The | (Molar | (Molar |
| No. | (° C.) | (h) | reductant | Ratio) | Ratio) |
| E111 | 4 | 4 | TCEP-NO | 9:1 | 0.22:1 |
| E112 | 4 | 4 | TCEP-NO | 10:1 | 0.1:1 |
| E113 | 4 | 6 | TCEP-NO | 10:1 | 0.1:1 |
| E114 | 4 | 8 | TCEP-NO | 10:1 | 0.1:1 |
| E115 | 4 | 10 | TCEP-NO | 10:1 | 0.1:1 |
| E116 | 4 | 22 | TCEPA | 3.5:1 | 0.57:1 |
| E117 | 10 | 16 | TCEPA | 3.2:1 | 0.63:1 |
| E118 | 15 | 16 | TCEPA | 3.5:1 | 0.57:1 |
| E119 | 25 | 6 | TCEPA | 3.9:1 | 0.51:1 |

The homogeneity assays results were shown as follows:

TABLE 7

| | | The reduction | | | | | |
|---|---|---|---|---|---|---|---|
| | | temperature | | | | | |
| | | and time in | D 0 | D 2 | D 4 | D 6 | D 8 |
| No. | FIG. | step (1) | (%) | (%) | (%) | (%) | (%) |
| E111 | 38A | 4° C., 4 h | 0 | 4.12 | 5.79 | 77.34 | 12.75 |
| E112 | 38B | 4° C., 4 h | 0 | 3.49 | 9.03 | 85.31 | 2.17 |
| E113 | 38C | 4° C., 6 h | 0 | 2.00 | 5.23 | 88.91 | 3.86 |
| E114 | 38D | 4° C., 8 h | 0 | 0 | 3.19 | 88.59 | 8.22 |
| E115 | 38E | 4° C., 10 h | 0 | 0 | 2.09 | 85.57 | 12.34 |
| E116 | 38F | 4° C., 22 h | 0 | 4.42 | 7.84 | 87.75 | 0 |
| E117 | 39A | 10° C., 16 h | 0 | 2.82 | 8.48 | 84.42 | 4.28 |
| E118 | 39B | 15° C., 16 h | 0 | 5.35 | 8.07 | 84.71 | 1.87 |
| E119 | 39C | 25° C., 6 h | 1.05 | 2.63 | 5.76 | 75.35 | 15.22 |

As shown in examples 111-116 in table 7, the results showed the content of D6 is up to 75%, even to 80, 85 or 88% when the molar ratio of the reductant and the antibody is 3.5:1, 9:1 or 10:1 and the reductant time in step (1) is from 4 h to 22 h, which also indicates that increasing the molar ratio of the reductant and the antibody, the method displayed here is with less reduction time cost.

As shown in examples 116-119 in table 7, the results showed the content of D6 is up to 75%, 85%, even to 87% when the reductant temperature in step (1) is from 4° C. to 25° C.

### Example 120: Preparation of Trastuzumab-[Bismaleimide-DBCO]₃ Conjugate

(1) ZnCl₂ (0.024 mM) and reductant TCEPA (0.048 mM) were added to a solution of a monoclonal antibody Trastuzumab (0.012 mM, in BES buffer, pH7.0, 20 mM) and the reaction mixture was allowed to stay at 4° C. for 18 h;
(2) EDTA-2Na (0.6 mM) and Bismaleimide-DBCO (0.045 mM) in DMA was introduced and the reaction was continued at room temperature for 1 h; then recovering Trastuzumab-[Bismaleimide-DBC]₃ using a desalting column.

Wherein, Bismaleimide-DBCO was prepared as follows:

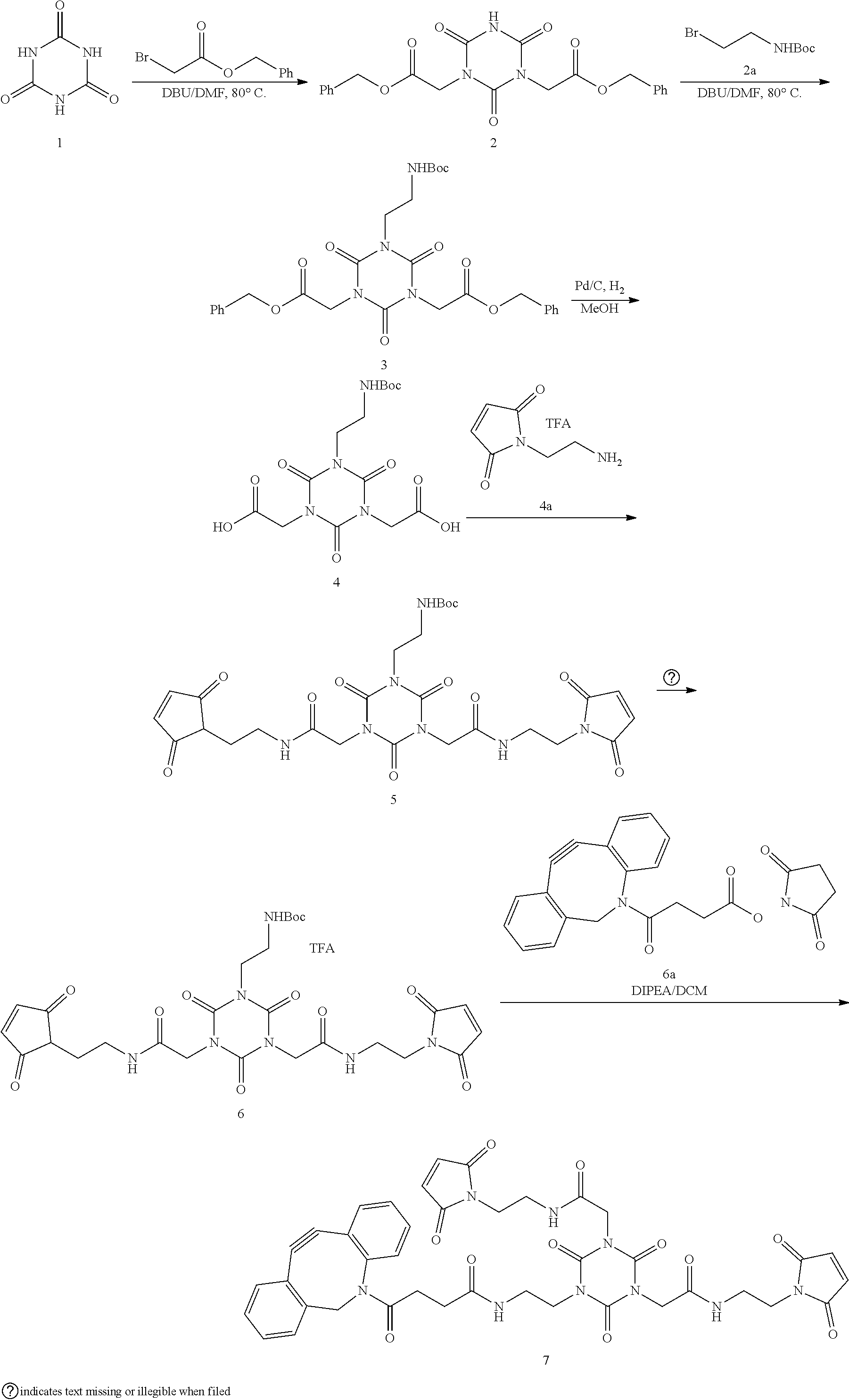

Intermediate 2: To a mixture of 1 (6.5 g, 50.0 mmol, 1.0 eq) in DMF (100 mL) was added DBU (30.7 g, 0.2 mol, 4.0 eq). The mixture was stirred for 1 h at 80° C., and Benzyl 2-bromoacetate (25.4 g, 0.11 mol, 2.2 eq). The resulting mixture was stirred for 16 h at 80° C. The mixture was poured into ice-water (600 mL), extracted with EtOAc (200 mL*3). The combined organic layer was washed with brine (200 mL), dried over Na₂SO₄ and filtered. The filtrate was concentrated, and purified by flash column (EtOAc/petroleum ether=0˜40%) to give product 2 (4.5 g, 21.0%) as white solid.

Intermediate 3: To a mixture of intermediate 2 (4.5 g, 10.58 mmol, 1.0 eq) in DMF (50 mL) was added DBU (3.22 g, 21.16 mmol, 2.0 eq). The mixture was stirred for 0.5 h at 80° C., and tert-Butyl N-(2-bromoethyl) carbamate (3.56 g, 15.87 mmol, 1.5 eq). The resulting mixture was stirred for 6 h at 80° C. TLC showed compound 1 was consumed completely. The mixture was poured into ice-water (300 mL), extracted with EtOAc (100 mL*3). The combined organic layer was washed with brine (100 mL), dried over Na₂SO₄ and filtered. The filtrate was concentrated, and purified by flash column (EtOAc/petroleum ether=0˜40%) to give product 3 (4.8 g, 79.8%) as white solid.

Intermediate 4: To a mixture of intermediate 3 (4.8 g, 8.44 mmol, 1.0 eq) in THF (50 mL) was added Pd/C (500 mg, 10% Pd/C, wetted with ca. 55% Water). The mixture was degassed 3 times and purged with H₂. The resulting mixture was stirred for 2 h at room temperature under H₂ atmosphere. TLC showed intermediate 3 was consumed completely. The mixture was filtered through a Celite pad and filtrate was concentrated to give product 4 (3.0 g, 91.5%) as white solid.

Intermediate 5: To a mixture of intermediate 4 (50 mg, 1.00 mmol, 1.0 eq) in DMF (5 mL) was added HATU (0.92 g, 2.4 mmol, 2.4 eq) and DIPEA (0.65 g, 5.0 mmol, 5.0 eq). The mixture was stirred for 0.5 h at room temperature, and 4a (0.51 g, 2.0 mmol, 2.0 eq) was added. The resulting mixture was stirred for 2 h at room temperature. LCMS showed intermediate 4 was consumed completely. The mixture was quenched by adding HCl (0.5M, 2 mL), and purified by RP-column (water/MeCN=10˜70%), and the eluent was lyophilized to give product 5 (0.51 g, 80.3%) as white solid.

Intermediate 6: To a mixture of intermediate 5 (100 mg, 0.16 mmol, 1.0 eq) in DCM (2 mL) was added TFA (100 uL). The mixture was stirred for 1 hours at room temperature. LCMS showed intermediate 5 was consumed completely. The mixture was concentrated and the residue was taken up by water (10 mL), then lyophilized to give product 6 (100 mg, 97.8%) as white solid.

Compound 7 (Bismaleimide-DBCO): To a mixture of intermediate 6 (50 mg, 93.9 umol, 1.0 eq) in DCM (2 mL) was added 6a (37.8 mg, 93.9 umol, 1.0 eq) followed by DIPEA (17 uL, 93.9 umol, 1.0 eq). The mixture was stirred for 1 h at room temperature. LCMS showed intermediate 6 was consumed completely. The mixture was concentrated and the residue was purified by prep-HPLC (water/MeCN=10˜50%), and the eluent was lyophilized to give product 7 (28.8 mg, 3.41%) as white solid. MS(M+H)⁺=820.19, exact mass calc. for C₄₀H₃₇N₉O₁₁ is 819.26. ¹H NMR (400 MHz, DMSO-d6): δ 8.17 (t, J=6.0 Hz, 2H), 7.85 (d, J=6.4 Hz, 1H), 7.64 (dd, J=18.4, 7.3 Hz, 2H), 7.47 (d, J=10.3 Hz, 3H), 7.38-7.24 (m, 3H), 6.96 (s, 4H), 5.02 (d, J=14.0 Hz, 1H), 4.22 (s, 4H), 3.68 (t, J=6.6 Hz, 2H), 3.65-3.58 (m, 4H), 3.43 (t, J=6.2 Hz, 3H), 3.24-3.20 (m, 3H), 3.16-3.10 (m, 5H).

The homogeneity assays results were shown as follows:

| NO. | FIG. | The type of the ADCs | D3 (%) | D4 (%) |
|---|---|---|---|---|
| E120 | 40 | D3 | 82.93 | 17.07 |

As shown in the table, the result demonstrated that the content of Trastuzumab-[Bismaleimide-DBCO]₃ was generally up to 82%, which indicated the process of method was benefit for site-specific modifying the antibody with D3 and improving the homogeneity.

### Example 121: Preparation of Trastuzumab-[MC-VC-PAB-MMAE]₆[MC-GGFG-DXd]₂ (The ADC with D6+D2)

(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4° C. for 18 h;
(2) EDTA-2Na (0.6 mM) and MC-VC-PAB-MMAE (0.08 mM) in DMA was introduced and the reaction was continued at room temperature for 1 h, then recovering the resulting product using a desalting column;
(3) The resulting product of step (2) and the second reductant TCEP (0.02 mM) were incubating at 37° C. for 3 h; a second linker-payload MC-GGFG-DXd (0.05 mM) added and the reaction was continued at room temperature for 2 h, then recovering the resulting product using a desalting column.

Wherein about six drug molecules MC-VC-PAB-MMAE were coupled to Trastuzumab on average, and about two drug molecules MC-GGFG-DXd were coupled to Trastuzumab on average.

The homogeneity assays results were shown as follows:

| | | D2 | D4 | D6 | D8 |
|---|---|---|---|---|---|
| NO. | FIG. | (%) | (%) | (%) | (%) |
| E121-step (2) | 41A | 4.48 | 4.97 | 86.49 | 4.06 |
| | | D2 | D4 | D6 + | D8 |
| NO. | FIG. | (%) | (%) | D2 (%) | (%) |
| E121-step (3) | 41B | 3.67 | 3.88 | 84.81 | 7.65 |

As shown the above table, the result demonstrated that the content of the ADC with D6+D2 was generally up to 84.81%, which indicated the process of method was benefit for site-specific modifying the antibody with D6+D2 and improving the homogeneity.

### Example 122: Preparation of Trastuzumab-[MC-GGFG-DXd]₆[Maleimide-PEG4-N3-DBCO-Cy3]₁ (The ADC with D6+D1)

1. Synthesis of dibromomaleimide-PEG4-N3

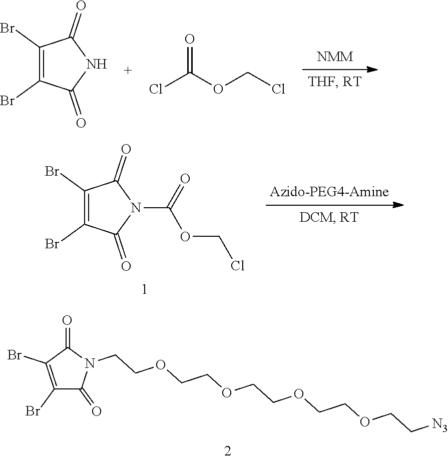 [SMILES: O=C1NC(=O)C(Br)=C1Br ; O=C(Cl)OCCl ; O=C(OCCl)N1C(=O)C(Br)=C(Br)C1=O ; [N-]=[N+]=NCCOCCOCCOCCOCCN1C(=O)C(Br)=C(Br)C1=O]

To a solution of 3,4-dibromomaleimide (127 mg, 0.5 mmol, 1 eq) and N-methylmorpholine (0.22 mL, 2 mmol, 4 eq) in THF (3.5 mL), chloromethyl chloroformate (0.18 mL, 2 mmol, 4 eq) was added and the mixture was stirred for 20 min at room temperature. Then DCM (10 mL) was added, the organic phase was washed with H₂O, dried over MgSO₄ and the solvent removed in vacuo to yield the title product 1 (139 mg, 0.4 mmol, 80%).

A solution of Azido-PEG4-Amine (105 mg, 0.4 mmol, 1 eq, Xi'an Confluore Biological Technology Co., Ltd) in dichloromethane (2 mL) was added to a stirred solution of product 1 (139 mg, 0.4 mmol, 1 eq) in dichloromethane (2 mL).

After 30 minutes, dichloromethane (6 mL) was added and the solution washed with a 0.68 M acetate buffer pH 5 (10 mL), water (1 mL), and dried with MgSO₄. Concentration in vacuo followed by purification by column chromatography (100% EtOAc as the mobile phase) yielded dibromomaleimide-PEG4-N3 (the title product 2) as a pale yellow oil (150 mg, 0.3 mmol, 75%).

2. Preparation of Trastuzumab-[MC-GGFG-DXd]₆[Maleimide-PEG4-N3-DBCO-Cy3]₁
(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4 T for 18 h;
(2) EDTA-2Na (0.6 mM) and MC-GGFG-DXd (0.096 mM) in DMA was introduced and the reaction was continued at room temperature for 1 h, then recovering the product using a desalting column;
(3) The resulting product of step (2) and the second reductant TCEP (0.02 mM) were incubating at room temperature for 2 h; a thio-bridging reagent [Dibromomaleimide-PEG4-N3] (0.012 mM) added and the reaction was continued at room temperature for 2 h; then a second linker-payload [DBCO-Cy3] (0.05 mM) added and the reaction was continued at room temperature for 4 h.
(4) purifying the resultant product using a desalting column.

Wherein about six drug molecules MC-GGFG-DXd were coupled to Trastuzumab on average, and about one drug molecule Maleimide-PEG4-N3-DBCO-Cy3 was coupled to Trastuzumab on average.

The homogeneity assays results were shown as follows:

| | | D4 | D6 | D8 |
|---|---|---|---|---|
| NO. | FIG. | (%) | (%) | (%) |
| E122-step (2) | 42A | 4.58 | 92.20 | 3.23 |
| | | D4 + D1 | D6 + D1 | D8 |
| NO. | FIG. | (%) | (%) | (%) |
| E122-step (4) | 42B | 4.26 | 92.25 | 3.50 |

As shown the above table, the result demonstrated that the content of the ADC with D6+D1 was generally up to 90%%, which indicated the process of method was benefit for site-specific modifying the antibody with D6+D1 and improving the homogeneity.

### Example 123: Preparation of Trastuzumab-[Maleimide]₆[MC-VC-PAB-MMAE]₂ (The ADC with D0+D2)

(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4° C. for 18 h;
(2) introducing EDTA (0.6 mM) and (2-Aminoethyl) maleimide (0.1 mM) to react with the reduced thiol groups resulted from step (1) at room temperature for 1 h, then recovering the product using a desalting column to afford Trastuzumab-[Maleimide]₆;
(3) Trastuzumab-[Maleimide]₆ and the second reductant TCEP (0.02 mM) were incubating at 37° C. for 18 h; a second linker-payload MC-VC-PAB-MMAE (0.05 mM) was added and the reaction was continued at room temperature for 2 h.
(4) purifying the resultant product using a desalting column.

The homogeneity assays results were shown as follows:

| NO. | FIG. | D0(%) | / | / |
|---|---|---|---|---|
| E123-step | 43A | 100 | / | / |
| (2) | | | | |
| | | D0 | D0 + | D0 + |
| NO. | FIG. | (%) | D1 (%) | D2 (%) |
| E123-step | 43B | 24.30 | 5.18 | 70.53 |
| (4) | | | | |

As shown in the above table, the result demonstrated that the content of the ADC with D0+D2 was generally up to 70.53%, which indicated the process of method was benefit for site-specific modifying the antibody with D0+D2 and improving the homogeneity.

### Example 124: Preparation of Trastuzumab-[Maleimide]₆[Maleimide-PEG4-N3-DBCO-Cy3]₁ (The ADC with D0+D1)

(1) ZnCl₂ (0.024 mM) and reductant TCEPA (0.048 mM) were added to a solution of a monoclonal antibody Trastuzumab (0.012 mM) in BES buffer (pH7.0, 20 mM) and the reaction mixture was allowed to stay at 4 TC for 18 h;
(2) introducing EDTA (0.6 mM) and (2-Aminoethyl) maleimide (0.1 mM) to react with reduced thiol groups resulted from step (1) at room temperature for 1 h, then recovering the product using a desalting column to afford Trastuzumab-[Maleimide]₆;
(3) Trastuzumab-[Maleimide]₆ and the second reductant TCEP (0.02 mM) were incubating at 37° C. for 18 h. the second thio-bridging reagent with the reactive group dibromomaleimide-PEG4-N3 (0.013 mM) to react with reduced thiol groups, the reaction temperature is 24° C. and the reaction time is 3 h;
(4) incubating resulting product and DBCO-Cy3 (0.02 mM) in MES (20 mM. pH6.7), the reaction temperature is 25° C. and the reaction time is 8 h, then recovering Trastuzumab-[Maleimide]₆[Maleimide-PEG4-N3-DBCO-Cy3] using a desalting column.

The homogeneity assays results were shown as follows:

| | | D0 | D0 + D1 | D0 + D2 |
|---|---|---|---|---|
| No. | FIG. | (%) | (%) | (%) |
| E124 | 44 | 17.00 | 79.40 | 3.60 |

As shown in the above table, the result demonstrated that the content of the ADC with D0+D1 was generally up to 79.40%, which indicated the process of method was benefit for site-specific modifying the antibody with D0+D1 and improving the homogeneity.

### Example 125: Preparation of Trastuzumab-[MC-VC-PAB-MMAE]₂ Conjugate (The ADC with D2)

(1) ZnCl₂ (0.012 mM) and reductant TCEPA prepared (0.048 mM) were added to a solution of a monoclonal antibody Trastuzumab (0.012 mM, in BES buffer, pH7.0, 20 mM) and the reaction mixture was allowed to stay at 4° C. for 18 h;
(2) DHAA (0.072 mM) was added to the mixture of step (1) and vortexed for mixing. The mixture was then incubated in darkness at room temperature for 2 h;
(3) optionally, purification with a desalting column to remove excess DHAA;
(4) EDTA (0.06 mM) and MC-VC-PAB-MMAE (0.048 mM) in DMA were added and the mixture was then incubated at room temperature for 1 h.
(5) The reaction mixture was subjected to purification using a de-salting column.

### Examples 126-147: Preparation of Trastuzumab-[MC-VC-PAB-MMVAE]₂ Conjugate (The ADC with D2)

The method of examples 126-147 is similar to example 125, and the difference is the parameters in step (1) and in step (2), the different parameters are shown as follows,

| | DHAA/mAb | Antibody | |
|---|---|---|---|
| No. | (Molar Ratio) | (mM) | Oxidation reaction |
| E126 | 8:1 | 0.012 | RT, 2 h |
| E127 | 10:1 | 0.012 | RT, 2 h |
| E128 | 12:1 | 0.012 | RT, 2 h |
| E129 | 14:1 | 0.012 | RT, 2 h |
| E130 | 8:1 | 0.012 | RT, 2 h and purification |
| | | | after oxidation |
| E131 | 8:1 | 0.012 | 4° C., 5 h |
| E132 | 10:1 | 0.012 | RT, 2 h and purification |
| | | | after oxidation |
| E133 | 10:1 | 0.012 | 4° C., 5 h |

RT is short for room temperature.

| | | Reductant/ | DHAA/mAb | | The time in |
|---|---|---|---|---|---|
| | | antibody | (Molar | Antibody | step (1) |
| No. | Reductant | (molar ratio) | Ratio) | (mM) | (h) |
| E134 | TCEP-NO | 4:1 | 4:1 | 0.012 | 2 |
| E135 | TCEP-NO | 4:1 | 4:1 | 0.012 | 4 |
| E136 | TCEP-NO | 4:1 | 6:1 | 0.012 | 4 |
| E137 | TCEP-NO | 4:1 | 8:1 | 0.012 | 2 |
| E138 | TCEP-NO | 4:1 | 8:1 | 0.012 | 4 |
| E139 | TCEP-NO | 4:1 | 10:1 | 0.012 | 4 |
| E140 | TCEP-NO | 6:1 | 7:1 | 0.012 | 4 |
| E141 | TCEP-NO | 6:1 | 10:1 | 0.012 | 4 |
| E142 | TCEP-NO | 6:1 | 13:1 | 0.012 | 4 |
| E143 | TCEP-NO | 6:1 | 16:1 | 0.012 | 4 |
| E144 | TCEP-NO | 10:1 | 10:1 | 0.012 | 4 |
| E145 | TCEP-NO | 10:1 | 14:1 | 0.012 | 4 |
| E146 | TCEP-NO | 10:1 | 18:1 | 0.012 | 4 |
| E147 | TCEP-NO | 10:1 | 22:1 | 0.012 | 4 |

The homogeneity assays results were shown as follows:

TABLE 8

| | | D 0 | D 2 | D 4 | D 6 | D 8 |
|---|---|---|---|---|---|---|
| No. | FIG. | (wt %) | (wt %) | (wt %) | (wt %) | (wt %) |
| E125 | 45 | 4 | 93 | 3 | 0 | 0 |
| E126 | 46 | 7 | 93 | 0 | 0 | 0 |
| E127 | 47 | 8 | 92 | 0 | 0 | 0 |
| E128 | 48 | 10 | 90 | 0 | 0 | 0 |
| E129 | 49 | 12 | 88 | 0 | 0 | 0 |
| E130 | 50 | 5 | 95 | 0 | 0 | 0 |
| E131 | 51 | 6 | 86 | 5 | 3 | 0 |
| E132 | 52 | 6 | 94 | 0 | 0 | 0 |
| E133 | 53 | 7 | 88 | 5 | 0 | 0 |
| E134 | 54A | 7.24 | 81.27 | 11.49 | 0 | 0 |
| E135 | 54B | 22.43 | 71.72 | 5.85 | 0 | 0 |
| E136 | 54C | 26.93 | 73.07 | 0 | 0 | 0 |
| E137 | 54D | 12.42 | 85.32 | 2.26 | 0 | 0 |
| E138 | 54E | 32.55 | 67.45 | 0 | 0 | 0 |
| E139 | 54F | 33.57 | 66.43 | 0 | 0 | 0 |
| E140 | 54G | 2.34 | 60.64 | 33.30 | 3.72 | 0 |
| E141 | 54H | 4.50 | 87.68 | 7.82 | 0 | 0 |
| E142 | 55A | 6.19 | 92.05 | 1.77 | 0 | 0 |
| E143 | 55B | 8.49 | 91.51 | 0 | 0 | 0 |
| E144 | 55C | 2.23 | 71.71 | 26.06 | 0 | 0 |
| E145 | 55D | 4.48 | 91.94 | 3.58 | 0 | 0 |
| E146 | 55E | 7.36 | 92.64 | 0 | 0 | 0 |
| E147 | 55F | 8.06 | 91.94 | 0 | 0 | 0 |

The results of Examples 125-147 were shown in Table 8. As the results shown in table 8, linker-payloads (MC-VC-PAB-MMAE) were successfully linked to Trastuzumab, which indicated ADCs with D2 were successfully synthesized.

According to the results of Table 8, The proportion of D2 in Example 125-133 was more than 80%. The results showed that the oxidation reaction was benefit for improving the homogeneity of ADCs with D2, and it also showed that oxidation reaction was benefit for antibody site-specific modification, especially benefit for ADCs with site-specific conjugation.

The proportion of D6 was up to 95% when performing purification after oxidation, which indicated that the purification could improve the homogeneity of ADCs with D2 significantly.

As shown in examples 134-147, the content of D2 is up to 60%, even to 70%, 80% and 90% when the molar ratio of the reductant and the antibody is from 4:1 to 10:1 and the reduction time in step (1) is shortened to 2 h or 4 h, which is with less reduction time cost. As shown in examples 125-147, when the reductant time in step (1) is 2 h to 18 h, the content of D2 is up to 60%, even to 70%, 80% and 90%.

As shown in example 125-147, the content of D2 is up to 60%, 70%, 80%, 85%, even to 90% or 95% when the molar ratio of DHAA and the antibody is 4:1 to 22:1.

### Examples 148-150: Preparation of Trastuzumab-[MC-VC-PAB-MMAE]₂ Conjugate (the Oxidation Temperature and/or Time in Step (2) is Different)

The method of examples 148-150 is similar to example 126, and the difference is the oxidation temperature and/or time in step (2) and the molar ratio of the reductant and the antibody, which are shown in table 9.

TABLE 9

| | | | The oxidation | | | | | |
|---|---|---|---|---|---|---|---|---|
| | | Reductant/ | temperature | | | | | |
| | | antibody | and time in | | | | | |
| No. | FIG. | (molar ratio) | step (2) | D 0(%) | D 2(%) | D 4(%) | D 6(%) | D 8(%) |
| E148 | 56A | 3.2:1 | 37° C., 1 h | 2.86 | 80.80 | 15.20 | 1.14 | 0 |
| E149 | 56B | 3.5:1 | 4° C., 24 h | 2.69 | 87.97 | 9.34 | 0 | 0 |
| E150 | 56C | 3.5:1 | 4° C., 48 h | 4.18 | 95.83 | 0 | 0 | 0 |

As shown in table 9 and in example 126, the results showed the content of D6 is up to 80%, even to 95% when the oxidation temperature in step (2) is from 4° C. to 37° C., and the oxidation time in step (2) is from 1 h to 48 h.

### Examples 151-166 and Comparative Example 6: Preparation of Trastuzumab-[MC-VC-PAB-MMAE]₂ Conjugate by Using the Different Buffer System

The method of examples 151-166 and comparative example 6 is similar to example 126, and the difference is the buffer system which is shown in table 9. Meanwhile, the molar ratio of the reductant and the antibody is 3.5:1 in examples 151-166 and comparative example 6.

The homogeneity assays results were shown as follows:

TABLE 10

| No. | FIG. | The buffer system | D 0(%) | D 2(%) | D 4(%) | D 6(%) | D 8(%) |
|---|---|---|---|---|---|---|---|
| E151 | 57A | Bis-Tris, pH 6.7 | 5.51 | 87.96 | 6.53 | 0 | 0 |
| E152 | 57B | PIPES, pH 6.7 | 4.46 | 79.81 | 15.73 | 0 | 0 |
| E153 | 57C | PB, pH 6.7 | 38.34 | 61.67 | 0 | 0 | 0 |
| E154 | 57D | HEPES, pH 7.0 | 6.06 | 91.51 | 2.43 | 0 | 0 |
| E155 | 57E | MOPS, pH 7.0 | 6.36 | 87.38 | 6.26 | 0 | 0 |
| E156 | 57F | DIPSO, pH 7.4 | 7.19 | 92.81 | 0 | 0 | 0 |
| E157 | 57G | MOBS, pH 7.4 | 6.18 | 93.83 | 0 | 0 | 0 |
| E158 | 57H | MOPSO, pH 7.4 | 5.70 | 92.18 | 2.12 | 0 | 0 |
| E159 | 58A | TES, pH 7.4 | 5.66 | 91.75 | 2.59 | 0 | 0 |
| E160 | 58B | ACES, pH 7.4 | 7.71 | 92.29 | 0 | 0 | 0 |
| E161 | 58C | TAPSO, pH 7.4 | 4.77 | 92.26 | 2.98 | 0 | 0 |
| E162 | 58D | MES pH 5.8 | 3.48 | 60.48 | 32.66 | 3.39 | 0 |
| E163 | 58E | MES pH 6.1 | 4.12 | 73.51 | 22.37 | 0 | 0 |
| E164 | 58F | BES, pH 6.4 | 3.57 | 76.45 | 19.98 | 0 | 0 |
| E165 | 58G | BES, pH 6.7 | 1.79 | 82.25 | 14.77 | 1.20 | 0 |
| E166 | 58H | BES, pH 7.4 | 3.05 | 94.48 | 2.48 | 0 | 0 |
| C6 | 59 | ADA, pH 6.7 | 86.47 | 13.53 | 0 | 0 | 0 |

As shown in table 10, the results showed the types and the pH value of the buffer system will impact the content of D2 by impacting the reduction kinetics and selectivity. The buffer systems of examples 151-166 are useful to increase the content of D2, and the pH value of the buffer system is from 5.8 to 7.4.

### Example 167: Preparation of Trastuzumab-[Maleimide-PEG4-N3-DBCO-Cy3]₁ (the ADC with D1)

(1) TCEPA (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 18 h;
(2) Adding DHAA (0.096 mM) to selectively re-oxidize the reduced thiol groups in Fab region resulted from step (1) at 25° C. for 2 h;
(3) Introducing EDTA (0.6 mM) and a first thio-bridging reagent dibromomaleimide-PEG4-N3 (0.013 mM) to react with reduced thiol groups resulted from step (3), the reaction temperature is 25° C. and the reaction time is 1 h, then recovering the product using a desalting column to afford Trastuzumab-[Maleimide-PEG4-N3]₁;
(4) incubating Trastuzumab-[Maleimide-PEG4-N3]1 (0.013 mM) and DBCO-Cy3 (0.02 mM) in BES buffer (20 mM, pH7.0), the reaction temperature is 25° C. and the reaction time is 8 h.
(5) The reaction mixture was subjected to purification using a de-salting column.

The homogeneity assays results were shown as follows:

| No. | FIG. | D0(%) | D1(%) | D2(%) |
|---|---|---|---|---|
| E167 | 60 | 13.07 | 83.81 | 3.13 |

As shown in the above table, the result demonstrated that the content of the ADC with D1 was generally up to 83.81%, which indicated the process of method was benefit for site-specific modifying the antibody with D1 and improving the homogeneity.

### Example 168: Preparation of Trastuzumab-[Maleimide-PEG4-N3-DBCO-Cy3]₁[MC-VC-PAB-MMAE]₆ (the ADC with D1+D6)

(1) introducing Trastuzumab-[Maleimide-PEG4-N3-DBCO-Cy3]₁ prepared from example 167 (0.008 mM) and TCEPA (0.08 mM) in BES buffer (20 mM, pH7.0), the reaction temperature is 24° C. and the reaction time is 24 h:
(2) introducing MC-VC-PAB-MMAE (0.14 mM) to solution from step (1), and the reaction mixture was allowed to stay at 24° C. for 1 h, then recovering Trastuzumab-[Maleimide-PEG4-N3-DBCO-Cy3]₁[MC-VC-PAB-MMAE]₆ using a desalting column.

The homogeneity assays results were shown as follows:

| No. | FIG. | D6(%) | D1 + D6 (%) | D2 + D6 (%) |
|---|---|---|---|---|
| E168 | 61 | 7.79 | 88.02 | 4.20 |

As shown in the above table, the result demonstrated that the content of the ADC with D1+D6 was generally up to 88.02%, which indicated the process of method was benefit for site-specific modifying the antibody with D1+D6 and improving the homogeneity.

### Example 169: Preparation of Trastuzumab-[Maleimide-PEG4-N3-DBCO-Cy3]₁[MC-VC-PAB-MMAE]₂ (the ADC with D1+D2)

(1) incubating ZnCl₂ (0.8 mM), the second reductant TCEP-6 (0.0144 mM) and Trastuzumab-[Maleimide-PEG4-N3-DBCO-Cy3]₁ prepared from example 167 (0.008 mM) in BES buffer (pH7.0, 20 mM) and the reaction mixture was allowed to stay at 4° C. for 4 h;
(2) introducing EDTA (3 mM) to trap Zn²⁺, and introducing MC-VC-PAB-MMAE (0.048 mM) to react with the reduced thiol groups resulted from step (2), the reaction temperature is 25° C. and the reaction time is 2 h;
(3) the reaction mixture was subjected to purification using a desalting column.

The homogeneity assays results were shown as follows:

| No. | FIG. | D 1(%) | D 2 (%) | D 1 + D 2 (%) | D 4% | D 1 + D 4 (%) |
|---|---|---|---|---|---|---|
| E169 | 62 | 3.72 | 7.92 | 73.80 | 4.06 | 10.51 |

With step (1), one of the interchain disulfide bonds in the ADC with D1 was reduced. As shown in the above table, the result demonstrated that the content of the ADC with D1+D2 was generally up to 73.8%, which indicated the process of method was benefit for site-specific modifying the antibody with D1+D2 and improving the homogeneity.

### Examples 170-171: Preparation of Trastuzumab-[Maleimide-PEG4-N3-DBCO-Cy3]₁[MC-VC-PAB-MMAE]₄ (the ADC with D1+D4)

(1) TCEPA (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 14 h;
(2) Adding DHAA (0.096 mM) to selectively re-oxidize the reduced thiol groups mainly in Fab region resulted from step (1) at 25° C. for 2 h;
(3) Introducing EDTA (0.6 mM) and a first thio-bridging reagent dibromomaleimide-PEG4-N3 (0.013 mM) to react with the reduced thiol groups resulted from step (3), the reaction temperature is 25° C. and the reaction time is 1.5 h, then recovering the product using a desalting column to afford Trastuzumab-[Maleimide-PEG4-N3]₁;
(4) incubating Trastuzumab-[Maleimide-PEG4-N3]₁ from step (3) and DBCO-Cy3 (0.02 mM) in BES buffer (20 mM, pH7.0), the reaction temperature is 25° C. and the reaction time is 8 h;
(5) The reaction mixture was subjected to purification using a de-salting column;
(6) incubating ZnCl₂ (0.8 mM), the second reductant TCEP-3 (0.0384 mM) or TCEP-6 (0.0336 mM) and the product from step (5) in BES buffer (pH7.0, 20 mM) and the reaction mixture was allowed to stay at 4° C. for 16 h;
(7) introducing EDAT (3 mM) to trap Zn²⁺, and introducing MC-VC-PAB-MMAE (0.08 mM) to react with the reduced thiol groups resulted from step (6), the reaction temperature is 25° C. and the reaction time is 2 h;
(8) the reaction mixture was subjected to purification using a desalting column.

The homogeneity assays results were shown as follows:

| No. | FIG. | Reductant | D 1(%) | D 2 (%) | / | / |
|---|---|---|---|---|---|---|
| E170-step (5) | 63A | TCEPA | 89.43 | 10.57 | / | / |
| No. | FIG. | Reductant | D 2(%) | D 1 + D 4 (%) | D 6(%) | D 1 + D 6 (%) |
| E170-step (8) | 63B | TCEP-3 | 2.01 | 84.48 | 2.83 | 10.69 |
| E171-step (8) | 63C | TCEP-6 | 3.74 | 84.17 | 2.38 | 9.71 |

With step (6), two of the interchain disulfide bonds in the ADC with D1 were reduced. As shown in the above table, the result demonstrated that the content of the ADC with D1+D4 was generally up to 84%, which indicated the process of method was benefit for site-specific modifying the antibody with D1+D4 and improving the homogeneity.

### Example 172: Preparation of Trastuzumab-[MC-GGFG-DXd]₂[MC-VC-PAB-MMAE]₄ Conjugate (The ADC with D2+D4)

(1) TCEPA (0.048 mM) and ZnCl₂ (0.012 mM) were added to a solution of Trastuzumab (0.012 mM) in BES buffer (20 mM, pH7.0) and the reaction mixture was vortexed for mixing, then the reaction mixture was incubated at 4° C. for 16 h;
(2) Adding DHAA (0.096 mM) to selectively re-oxidize the reduced thiol groups in Fab region resulted from step (1) at 25° C. for 2 h;
(3) Introducing EDTA (0.6 mM) and MC-GGFG-DXd (0.048 mM) in DMA to react with remained thiol groups from step (2) and the reaction was incubated at room temperature for 1.5 h;
(4) The reaction mixture was subjected to purification using a de-salting column;
(5) incubating ZnCl₂ (0.8 mM), the second reductant TCEP-3 (0.072 mM) and the product from step (4) in BES buffer (pH7.0, 20 mM) and the reaction mixture was allowed to stay at 4° C. for 16 h;
(6) introducing EDTA (3 mM) to trap Zn²⁺, and introducing MC-VC-PAB-MMAE (0.08 mM) to react with the reduced thiol groups resulted from step (5), the reaction temperature is 25° C. and the reaction time is 2 h;
(7) the reaction mixture was subjected to purification using a desalting column.

### Examples 173-174: Preparation of Trastuzumab-[MC-VC-PAB-MMAE]₂[MC-GGFG-DXd]₄ Conjugate (The ADC with D2+D4)

(1) TCEPA (0.042 mM) and ZnCl₂ (0.024 mM) were added to a solution of Trastuzumab (0.012 mM) in BES buffer (20 mM, pH7.0) and the reaction mixture was vortexed for mixing, then the reaction mixture was incubated at 4° C. for 16 h:
(2) Adding DHAA (0.12 mM) to selectively re-oxidize the reduced thiol groups in Fab region resulted from step (1) at 25° C. for 2 h;
(3) Introducing EDTA (0.12 mM) and MC-VC-PAB-MMAE (0.048 mM) in DMA to react with remained thiol groups from step (2) and the reaction was incubated at room temperature for 1.5 h;
(4) The reaction mixture was subjected to purification using a de-salting column;
(5) incubating ZnCl₂ (example 173: 0.36 mM; example 174: 0.024 mM), the second reductant TCEP-6 (example 173: 0.042 mM; example 174: 0.054 mM) and the product from step (4) in BES buffer (pH7.0, 20 mM) and the reaction mixture was allowed to stay at 3° C. for 1 h;
(6) introducing EDTA (1.2 mM) to trap Zn²⁺, and introducing MC-GGFG-DXd (0.096 mM) to react with the reduced thiol groups resulted from step (5), the reaction temperature is 25° C. and the reaction time is 2 h;
(7) the reaction mixture was subjected to purification using a desalting column.

The homogeneity assays results were shown as follows:

| No. | FIG. | Reductant | D 1(%) | D 2 (%) | D 4 (%) | D 6 (%) |
|---|---|---|---|---|---|---|
| E172-step (4) | 64A | TCEPA | 11.30 | 85.66 | 2.54 | 0.51 |
| No. | FIG. | Reductant | D 0(%) | D 2 (%) | D 4 (%) | D 6 (%) |
| E173-step (4) | 65A | TCEPA | 2.60 | 91.38 | 6.02 | 0 |
| E174-step (4) | / | TCEPA | 2.59 | 91.25 | 6.16 | 0 |
| | | | D 4(D × d) + D | D 2(D × d) + D | | |
| No. | FIG. | Reductant | 2 (MMAE) (%) | 4 (MMAE) (%) | D 6 (%) | / |
| E172-step (7) | 64B | TCEP-3 | 2.58 | 82.23 | 15.19 | / |
| No. | FIG. | Reductant | D 2 + D 2 (%) | D 2 + D 4 (%) | D 2 + D 6 (%) | D 8 (%) |
| E173-step (7) | 65B | TCEP-6 | 1.5 | 81.7 | 14.2 | 2.6 |
| E174-step (7) | 65C | TCEP-6 | 4.41 | 62.84 | 29.88 | 2.87 |

As shown in the above table, the result demonstrated that the content of the ADC with D2+D4 was generally up to 60%, which indicated the process of method was benefit for site-specific modifying the antibody with D2+D4 and improving the homogeneity.

As shown in examples 173-174, the result demonstrated that the selective reduction ability increases in step (5) when the molar ratio of the transition metal ions and the reductant increases in step (5).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1. A reductant having the following formula (I): 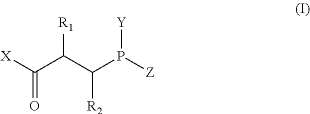
or a salt, solvate, stereoisomer thereof, which characterized in that,
R₁ is H, —NH₂, —C(O)(R₃R₄), unsubstituted or substituted C₁-C₈alkyl group, unsubstituted or substituted C₁-C₅ hydroxyalkyl group, or unsubstituted or substituted aryl group;
R₃ is N, NH or O;
R₄ is H, unsubstituted or substituted C₁-C₅alkyl group, unsubstituted or substituted C₁-C₅ hydroxyalkyl group, or unsubstituted or substituted aryl group;
R₂ is H, unsubstituted or substituted C₁-C₅ alkyl group, or unsubstituted or substituted C₁-C₅ hydroxyalkyl group;
X is OH, unsubstituted or substituted C₁-C₅alkoxy group or —NR₅R₆,
R₅ and R₆ independently are H, C₀-C₅ hydroxyalkyl group, unsubstituted or substituted C₁-C₅alkyl group, unsubstituted or substituted C₂-C₈ carboxy alkyl group, unsubstituted or substituted C₁-C₅ alkoxy group, unsubstituted or substituted heteroaryl alkyl group, unsubstituted or substituted aryl alkoxy group, unsubstituted or substituted arylalkyl group, unsubstituted or substituted aryl group, C₁-C₅alkyl sulfonyl group, or —(CH₂)n¹(OCH₂CH₂O)n²CH(R₈)CO(R₇),
R₇ is C₀-C₅ hydroxyalkyl group, —NHOH,
R₈ is H, unsubstituted or substituted arylalkyl group,
n¹ and n² independently are the number 0, 1, 2, 3, 4, and
Y is the same as X, or Y is an ester or amide of X,
Z is the same as X or Y, or
Y and Z independently are selected from the group consisting of 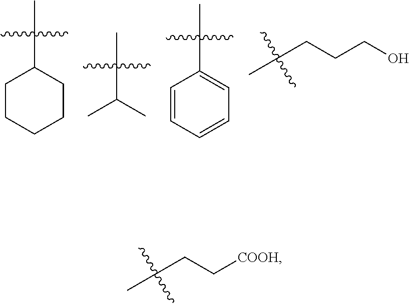
X, Y and Z are not 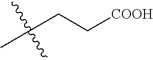 [SMILES: *CCC(=O)O]
at the same time.

2. The reductant of claim 1, which characterized in that,
R₁ is H, and R₂ is H.

3. The reductant of claim 1, which characterized in that,
X is —OCH₃, —OCH₂CH₃, or —OCH(CH₃)₂.

4. The reductant of claim 1, which characterized in that,
X is —NR₅R₆,
R₅ is H, and
R₆ is H, C₀-C₅ hydroxyalkyl group, C₁-C₅ alkoxy group, unsubstituted or substituted heteroaryl alkyl group, unsubstituted or substituted aryl alkoxy group, unsubstituted or substituted aryl group, unsubstituted or substituted arylalkyl group, C₁-C₅ alkyl sulfonyl group or —(CH₂)n¹(OCH₂CH₂O)n²CH(R₈)CO(R₇),
R₇ is C₀-C₃ hydroxyalkyl group or —NHOH,
R₈ is H or unsubstituted or substituted arylalkyl group,
n¹ and n² independently are the number 0.

5. The reductant of claim 4, which characterized in that,
R₆ is H, C₀-C₂ hydroxyalkyl group, C₁-C₃ alkoxy group, C₁-C₃ alkyl sulfonyl group, bipyridyl group, benzyl group, aryl alkoxy group, phenyl group which is unsubstituted or substituted with OH, carboxy or pyridyl group, or —CH(R₈)CO(R₇),
R₇ is OH or —NHOH,
R8 is H or benzyl group which is unsubstituted or substituted with —OH, halogen, cyano group or nitro group.

6. The reductant of claim 1, which characterized in that,
X is —NR₅R₆,
R₅ is H, and
R₆ is H, OH, —CH₂OH, —(CH₂)₂OH, —CH₃, —CH₂CH₃, —CH₂COOH, —(CH₂)₂COOH, —(CH₂)₃COOH, —(CH₂)₄COOH, —(CH₂)₅COOH, —OCH₃, —OCH₂CH₃, —CH₂CONHOH, —OC(C₆H₅)₃,

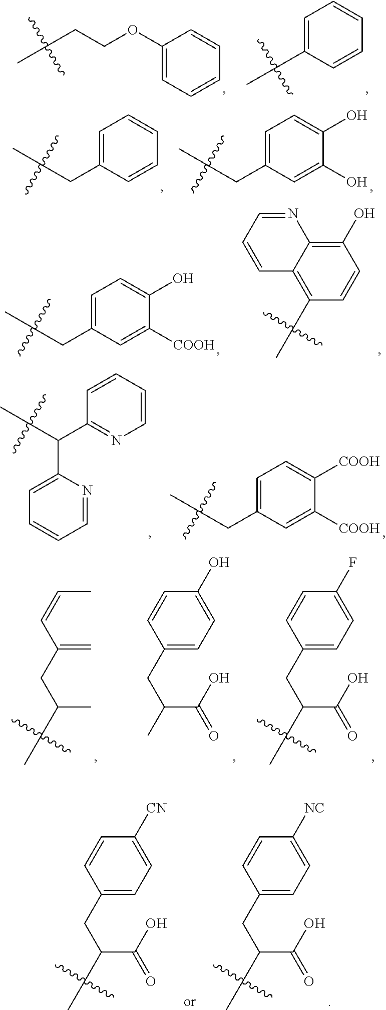

7. The reductant of claim 1, which characterized in that,
X is —NR5R6,
R5 is OH,
R6 is C1-C5 alkyl group, unsubstituted or substituted heteroaryl alkyl group, unsubstituted or substituted arylalkyl group, unsubstituted or substituted aryl group, or —(CH₂)n¹(OCH₂CH₂O)n²CH(R₈)CO(R₇),
R7 is C₀-C₅ hydroxyalkyl group,
R8 is H,
n¹ and n² independently are the number 0, 1, 2, 3, 4.

8. The reductant of claim 7, which characterized in that,
R₆ is C₁-C₃ alkyl group, heteroaryl alkyl group which comprises a heteroatom N, unsubstituted or substituted benzyl group, unsubstituted or substituted phenyl group, or —CH(R₈)CO(R₇),
R₇ is C₀-C₃ hydroxyalkyl group,
R₈ is H.

9. The reductant of claim 7, which characterized in that,
R₆ is —CH₃, —CH₂COOH,

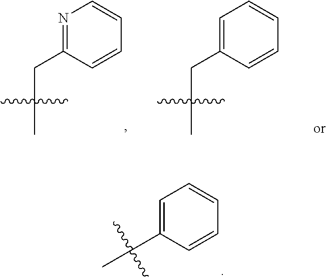 [SMILES: *Cc1ccccn1 ; *Cc1ccccc1 ; *c1ccccc1]

10. The reductant of claim 1, which characterized in that,
X is —NR₅R₆,
R₅ and R₆ independently are C₁-C₅ alkyl group, C₀-C₅ hydroxyalkyl group, unsubstituted or substituted heteroaryl alkyl group or —(CH₂)n¹(OCH₂CH₂O)n²CH(R₈)CO(R₇),
R₇ is C₀-C₅ hydroxyalkyl group or —NHOH,
R₈ is H,
n¹ and n² independently are the number 0, 1, 2, 3, 4.

11. The reductant of claim 10, which characterized in that,
R₅ and R₆ independently are methyl, ethyl group, —(CH₂)₂OH, —CH₂COOH, —CH₂CONHOH or

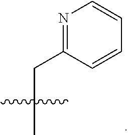 [SMILES: *Cc1ccccn1]

12. The reductant of claim 1, which characterized in that,
Y is 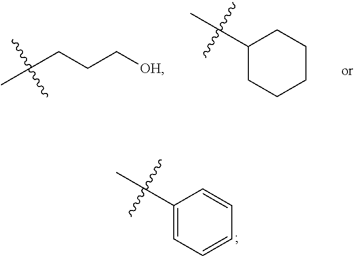 [SMILES: *CCCO ; *C1CCCCC1 ; *c1ccccc1]
and
Z is

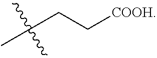 [SMILES: *CCC(=O)O]

13. The reductant of claim 1, which characterized in that, the reductant is selected from the group consisting of

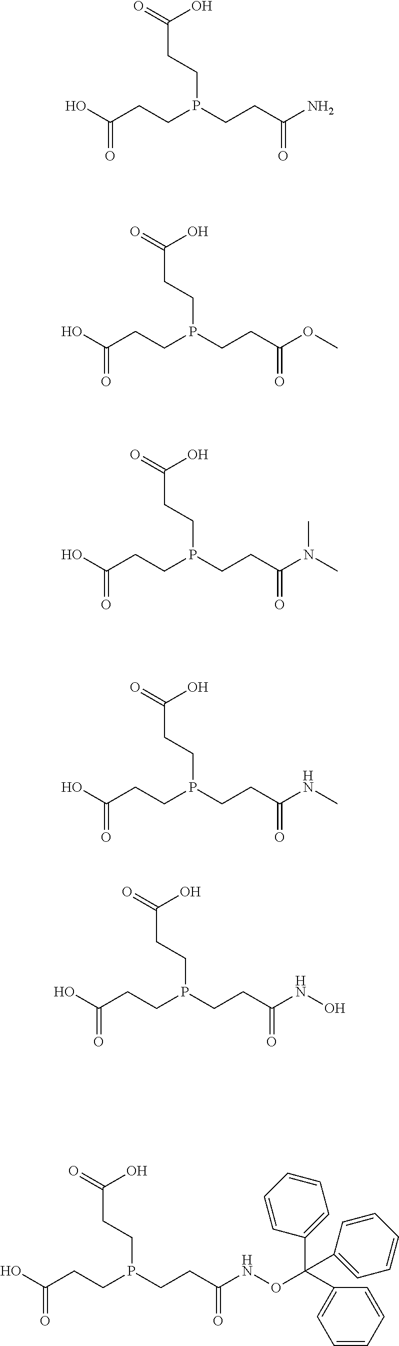 [SMILES: NC(=O)CCP(CCC(=O)O)CCC(=O)O ; COC(=O)CCP(CCC(=O)O)CCC(=O)O ; CN(C)C(=O)CCP(CCC(=O)O)CCC(=O)O ; CNC(=O)CCP(CCC(=O)O)CCC(=O)O ; O=C(O)CCP(CCC(=O)O)CCC(=O)NO ; O=C(O)CCP(CCC(=O)O)CCC(=O)NOC(c1ccccc1)(c1ccccc1)c1ccccc1]

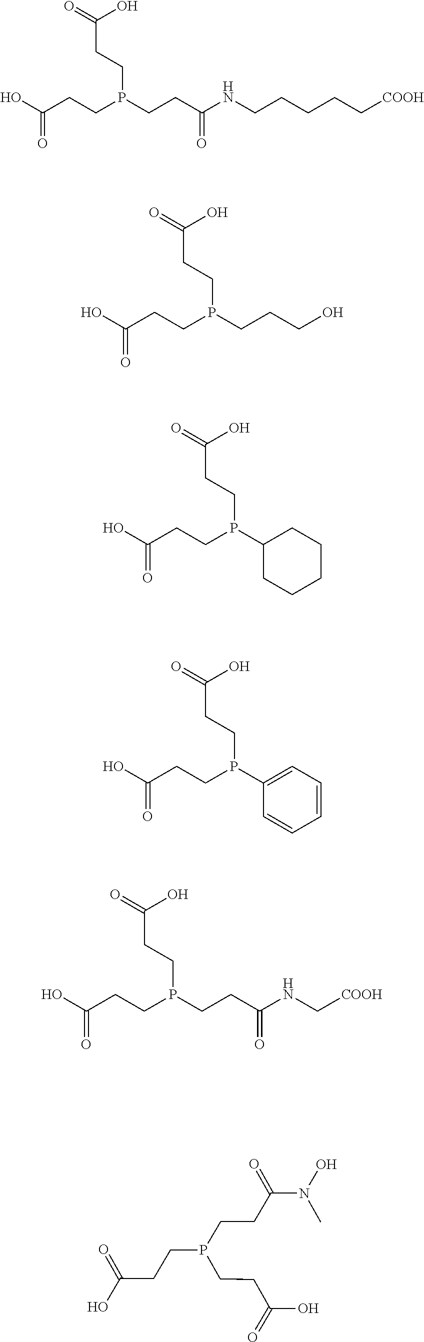 [SMILES: O=C(O)CCCCCNC(=O)CCP(CCC(=O)O)CCC(=O)O ; O=C(O)CCP(CCCO)CCC(=O)O ; O=C(O)CCP(CCC(=O)O)C1CCCCC1 ; O=C(O)CCP(CCC(=O)O)c1ccccc1 ; O=C(O)CCP(CCC(=O)O)CCC(=O)NCC(=O)O ; CN(O)C(=O)CCP(CCC(=O)O)CCC(=O)O]

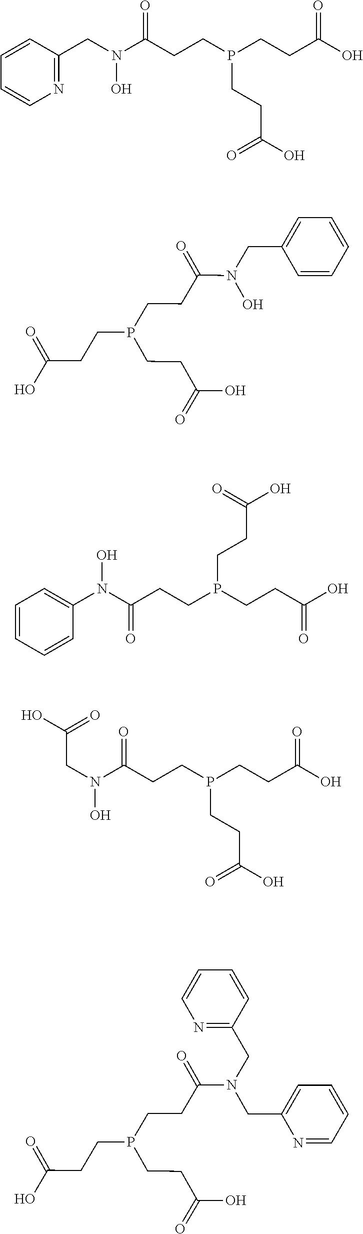 [SMILES: O=C(O)CCP(CCC(=O)O)CCC(=O)N(O)Cc1ccccn1 ; O=C(O)CCP(CCC(=O)O)CCC(=O)N(O)Cc1ccccc1 ; O=C(O)CCP(CCC(=O)O)CCC(=O)N(O)c1ccccc1 ; O=C(O)CCP(CCC(=O)O)CCC(=O)N(O)CC(=O)O ; O=C(O)CCP(CCC(=O)O)CCC(=O)N(Cc1ccccn1)Cc1ccccn1]

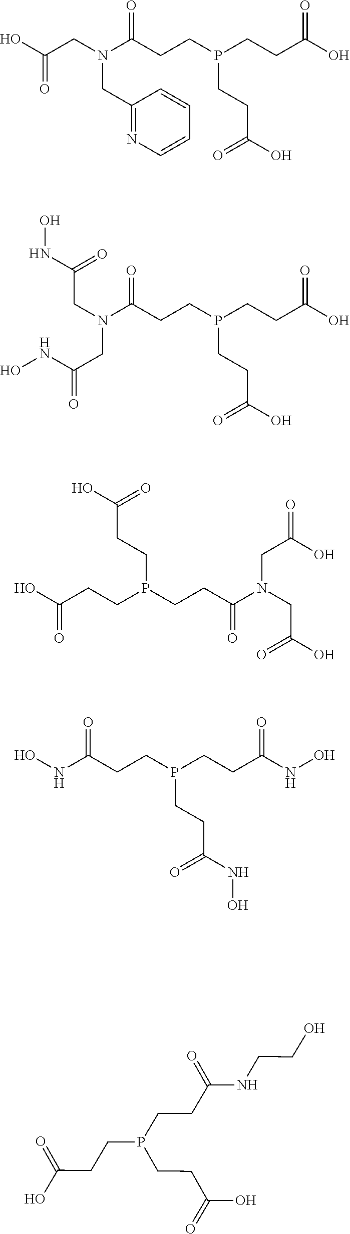 [SMILES: O=C(O)CCP(CCC(=O)O)CCC(=O)N(CC(=O)O)Cc1ccccn1 ; O=C(O)CCP(CCC(=O)O)CCC(=O)N(CC(=O)NO)CC(=O)NO ; O=C(O)CCP(CCC(=O)O)CCC(=O)N(CC(=O)O)CC(=O)O ; O=C(CCP(CCC(=O)NO)CCC(=O)NO)NO ; O=C(O)CCP(CCC(=O)O)CCC(=O)NCCO]

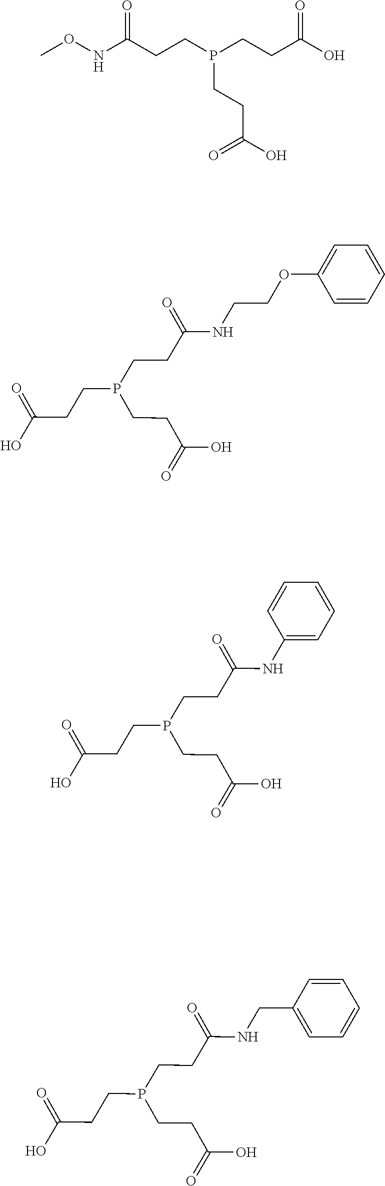 [SMILES: CONC(=O)CCP(CCC(=O)O)CCC(=O)O ; O=C(O)CCP(CCC(=O)O)CCC(=O)NCCOc1ccccc1 ; O=C(O)CCP(CCC(=O)O)CCC(=O)Nc1ccccc1 ; O=C(O)CCP(CCC(=O)O)CCC(=O)NCc1ccccc1]

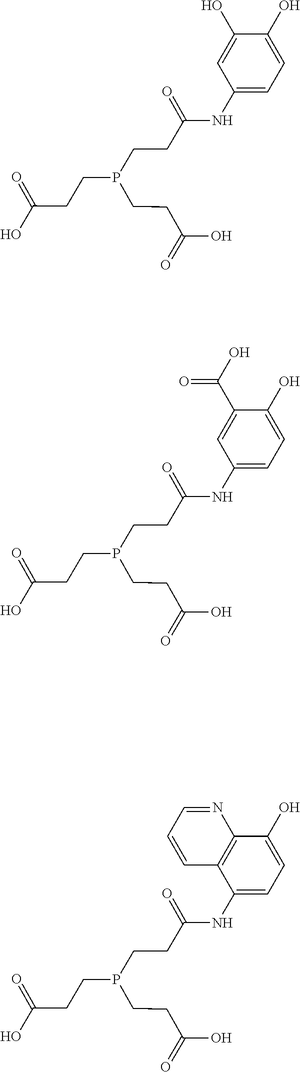 [SMILES: O=C(O)CCP(CCC(=O)O)CCC(=O)Nc1ccc(O)c(O)c1 ; O=C(O)CCP(CCC(=O)O)CCC(=O)Nc1ccc(O)c(C(=O)O)c1 ; O=C(O)CCP(CCC(=O)O)CCC(=O)Nc1ccc(O)c2ncccc12]

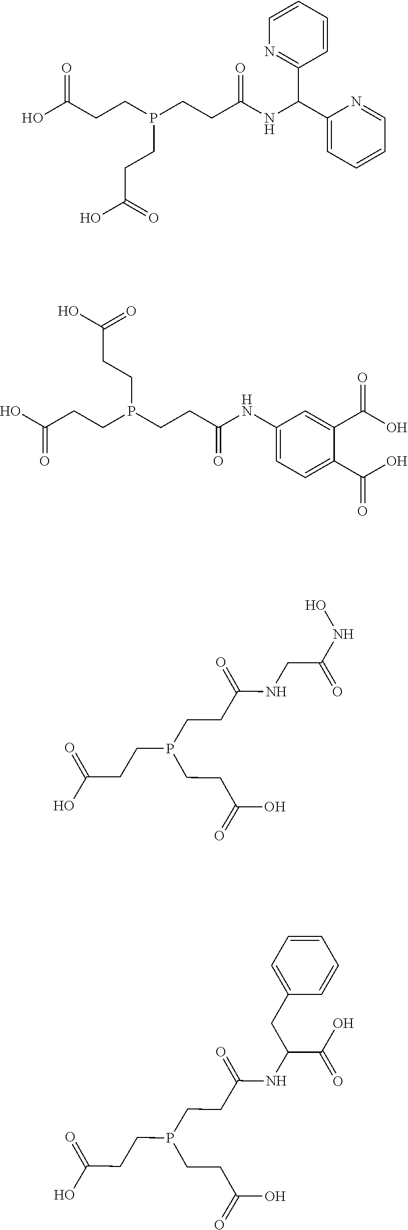 [SMILES: O=C(O)CCP(CCC(=O)O)CCC(=O)NC(c1ccccn1)c1ccccn1 ; O=C(O)CCP(CCC(=O)O)CCC(=O)Nc1ccc(C(=O)O)c(C(=O)O)c1 ; O=C(O)CCP(CCC(=O)O)CCC(=O)NCC(=O)NO ; O=C(O)CCP(CCC(=O)O)CCC(=O)NC(Cc1ccccc1)C(=O)O]

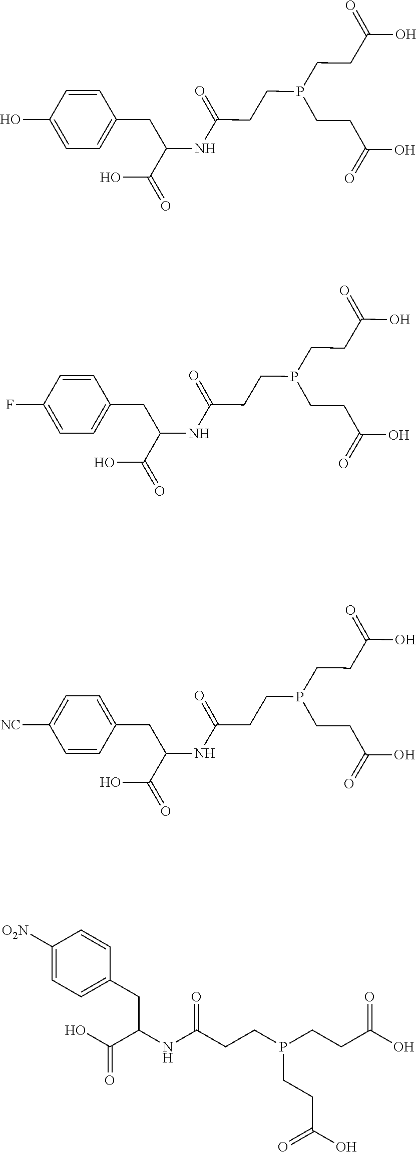 [SMILES: O=C(O)CCP(CCC(=O)O)CCC(=O)NC(Cc1ccc(O)cc1)C(=O)O ; O=C(O)CCP(CCC(=O)O)CCC(=O)NC(Cc1ccc(F)cc1)C(=O)O ; N#Cc1ccc(CC(NC(=O)CCP(CCC(=O)O)CCC(=O)O)C(=O)O)cc1 ; O=C(O)CCP(CCC(=O)O)CCC(=O)NC(Cc1ccc([N+](=O)[O-])cc1)C(=O)O]

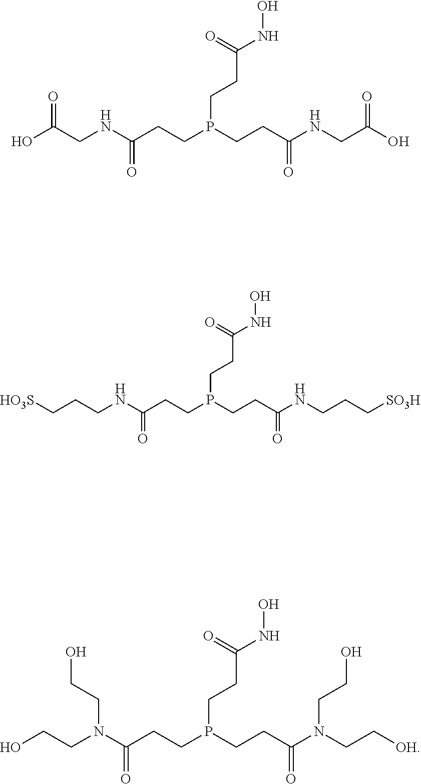 [SMILES: O=C(O)CNC(=O)CCP(CCC(=O)NO)CCC(=O)NCC(=O)O ; O=C(CCP(CCC(=O)NCCCS(=O)(=O)O)CCC(=O)NCCCS(=O)(=O)O)NO ; O=C(CCP(CCC(=O)N(CCO)CCO)CCC(=O)N(CCO)CCO)NO]

14. A composition comprising a reductant of claim 1 and transition metal ions.

15. The composition of claim 14, which characterized in that, the transition metal ions are Zn²⁺, Cd²⁺, Hg²⁺, Ni²⁺, Co²⁺ or the combination thereof, optionally, the transition metal ions are Zn²⁺.

16. The composition of claim 14, which characterized in that, the molar ratio of the transition metal ions and the reductant is 0.05:1 to 40:1, optionally, the molar ratio of the transition metal ions and the reductant is 0.25:1 to 30:1, more optionally, the molar ratio of the transition metal ions and the reductant is 0.25:1 to 15:1.

17. A method of preparing the reductant of claim 1, which characterized in that, at least one X′ is connected to a compound of formula II by introducing a condensation reagent under an inert atmosphere, 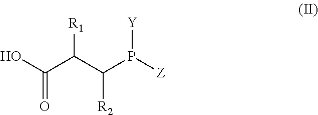
R₁ is H, —NH₂, —C(O)(R₃R₄), unsubstituted or substituted C₁-C₅ alkyl group, unsubstituted or substituted C₁-C₅ hydroxyalkyl group, or unsubstituted or substituted aryl group;
R₃ is N, NH or O;
R₄ is H, unsubstituted or substituted C₁-C₅ alkyl group, unsubstituted or substituted C₁-C₅ hydroxyalkyl group, or unsubstituted or substituted aryl group;
R₂ is H, unsubstituted or substituted C₁-C₅ alkyl group, or unsubstituted or substituted C₁-C₅ hydroxyalkyl group;
X′ is unsubstituted or substituted C₁-C₅ alkyl alcohol or NR₅R₆, R₅ and R₆ independently are H, C₀-C₅ hydroxyalkyl group, unsubstituted or substituted C₁-C₈alkyl group, unsubstituted or substituted C₂-C₈ carboxy alkyl group, unsubstituted or substituted C₁-C₅ alkoxy group, unsubstituted or substituted heteroaryl alkyl group, unsubstituted or substituted aryl alkoxy group, unsubstituted or substituted arylalkyl group, unsubstituted or substituted aryl group, C₁-C₈alkyl sulfonyl group, or —(CH₂)n¹(OCH₂CH₂O)n²CH(R₈)CO(R₇),
R₇ is C₀-C₅ hydroxyalkyl group, —NHOH,
R₈ is H, unsubstituted or substituted arylalkyl group,
N¹ and n² independently are the number 0, 1, 2, 3, 4, and
Y is the same as X, or Y is an ester or amide of X,
Z is the same as X or Y, or
Y and Z independently are selected from the group consisting of

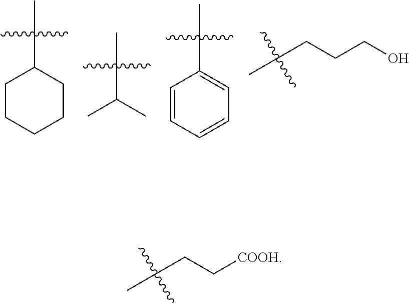

18. The method of claim 17, which characterized in that, the compound of formula II is 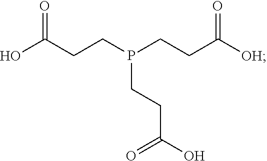 [SMILES: O=C(O)CCP(CCC(=O)O)CCC(=O)O]
and/or the X′ is 2-phenoxy-ethylamine, Phenylamine, Benzylamine, 4-Aminobenzene-1,2-diol, 5-Amino-2-hydroxybenzoic acid, Bis(pyridin-2-ylmethyl)amine, 5-Amino-8-hydroxyquinoline, Bis(pyridin-2-yl) methanamine, 4-Aminophthalic acid, tert-Butyl L-tyrosinate, DL-3-(4-Fluorophenyl)alanine, DL-4-Cyanophenylalanine, DL-4-nitro-phenylalanine, N-Benzylhydroxylamine hydrochloride, N-Phenylhydroxylamine,

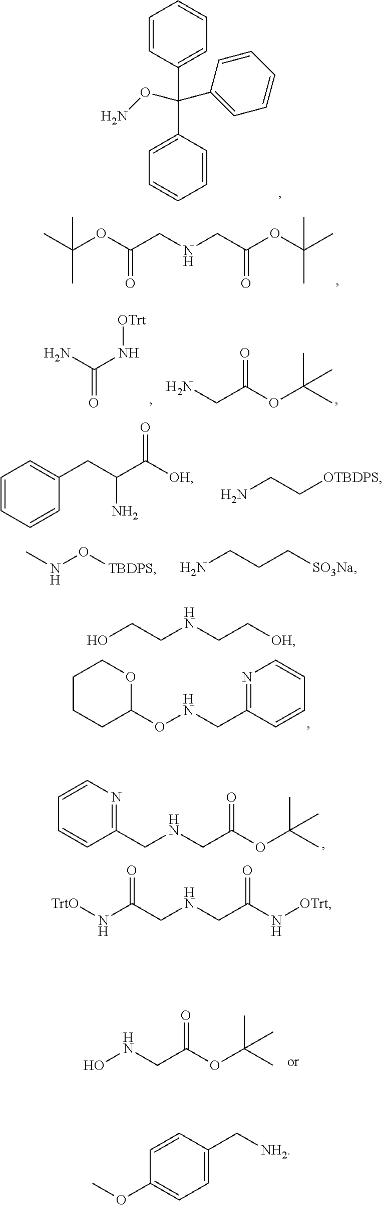

19.-21. (canceled)

22. A method of preparing an antibody with site-specific modification, comprising steps of
(A1) incubating a reductant of claim 1, a salt, solvate, stereoisomer thereof as a first reductant and the transition metal ions in the presence of an antibody in a buffer system to selectively the reduce interchain disulfide bonds within the antibody to afford the antibody bearing reduced thiol groups.

23. The method of claim 22, which characterized in that, two interchain disulfide bonds in Fab region of the antibody and one interchain disulfide bonds in hinge region of the antibody are reduced.

24. The method of claim 22, which characterized in that, the method further comprising step of
(A2) introducing oxidant to selectively re-oxidize the reduced thiol groups resulted from step (A1), optionally re-oxidize the reduced thiol groups in Fab region of the antibody.

25. The method of claim 24, which characterized in that, the method further comprising steps of
(A3) incubating a second reductant in a buffer system to selectively reduce the interchain disulfide bonds resulted from step (A2) to afford the antibody bearing the reduced thiol groups, optionally reduce the interchain disulfide bonds in the hinge region of the antibody.

26. The method of claim 22, which characterized in that, the method further comprising the following steps,
(B1) introducing metal chelators and first payload units to react with the reduced thiol groups resulted from step (A1), wherein, the first payload unit is an end capping reagent, a first linker-payload or a first thio-bridging reagent, optionally, the first thio-bridging reagent bears the first linker-payload or reactive groups;
or,
(B1) introducing metal chelators and first payload units to react with the reduced thiol groups resulted from step (A2), wherein, the first payload unit is an end capping reagent, a first linker-payload or a first thio-bridging reagent, optionally, the first thio-bridging reagent bears the first linker-payload or reactive groups, the step (A2) is that introducing oxidant to selectively re-oxidize the reduced thiol groups resulted from step (A1), optionally re-oxidize the reduced thiol groups in Fab region of the antibody;
or,
(B1) introducing metal chelators and first payload units to react with the reduced thiol groups resulted from step (A3), wherein, the first payload unit is an end capping reagent, a first linker-payload or a first thio-bridging reagent, optionally, the first thio-bridging reagent bears the first linker-payload or reactive groups, the step (A3) is that incubating a second reductant in a buffer system to selectively reduce the interchain disulfide bonds resulted from step (A2) to afford the antibody bearing the reduced thiol groups, optionally reduce the interchain disulfide bonds in the hinge region of the antibody.

27. The method of claim 26, which characterized in that, the method further comprising step of
(B2) incubating the second reductant in a buffer system to reduce interchain disulfide bonds resulted from step (B1), optionally, introducing the transition metal ions; and
(B3) introducing second payload units to react with the reduced thiol groups resulted from step (B2), optionally, introducing the metal chelators, wherein, the second payload unit is a second linker-payload or a second thio-bridging reagent, optionally, the second thio-bridging reagent bears the second linker-payload or reactive groups.

28. The method of claim 27, which characterized in that, the first thio-bridging reagent and the second thio-bridging reagent independently contain at least two substituted groups allowing a re-bridging of the thiol groups.

29. The method of claim 28, which characterized in that, the first thio-bridging reagent and the second thio-bridging are independently selected from the group consisting of

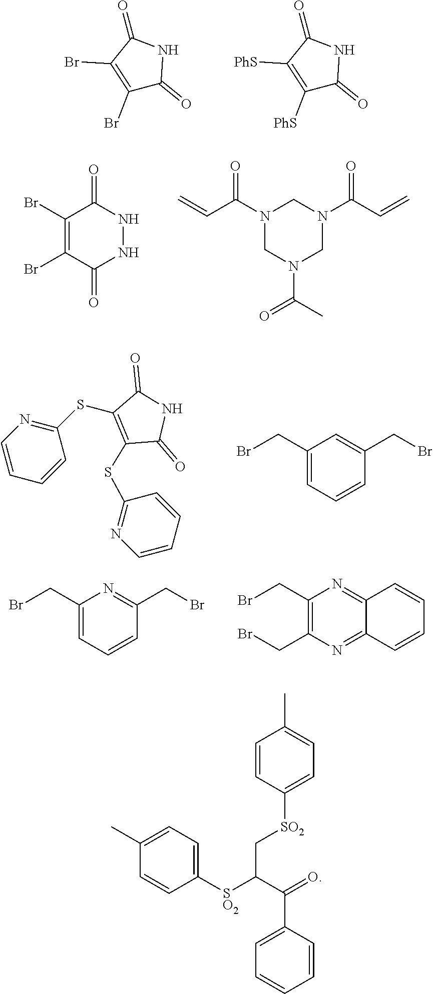

30. The method of any one of claims 22, 25 and 27, which characterized in that, the molar ratio of the reductant and the antibody in step (A1), (A3) and (B2) independently is 1:1 to 20, 1:1 to 5:1, 1:1 to 3:1, 1:1 to 2:1 or 3:1 to 5:1.

31. The method of claim 30, which characterized in that, the molar ratio of the first reductant and the antibody in step (A1) is 2.8:1 to 13:1, optionally, the molar ratio of the first reductant and the antibody is 3.5:1 to 5:1, 4:1 to 10:1 or 5:1 to 13:1.

32. The method of claim 27, which characterized in that, the incubation temperature in step (A1), (A3) and (B2) independently is 0° C. to 37° C., 0° C. to 25° C., 0° C. to 15° C., 0° C. to 10° C., or 0° C. to 5° C.; and/or the incubation time in step (A1) is 2 h to 24 h, 14 h to 24 h, 16 h to 20 h, or 16 h to 18 h; and/or
incubation time in step (A3) and (B2) independently is 0.5 h to 24 h, 0.5 h to 12 h, 1 h to 10 h, 1 h to 8 h, or 1 h to 5 h.

33. (canceled)

34. The method of claim 32, which characterized in that, in step (A1), the molar ratio of the first reductant and the antibody is 4:1 to 10:1, the incubation time is 1 h to 16 h; and/or in step (A1), the molar ratio of the first reductant and the antibody is 6:1 to 13:1, the incubation time is 4 h to 16 h/and/or in step (A1), the molar ratio of the first reductant and the antibody is 2.8:1 to 3:1, the incubation time is 10-24 h.

35-36. (canceled)

37. The method of claim 22, which characterized in that, the molar ratio of the transition metal ions and the first reductant in step (A1) is 0.05:1 to 40:1, 0.08:1 to 30:1, 0.1:1 to 20:1, 0.2:1 to 8:1, or 0.25:1 to 7.5:1.

38. The method of claim 27, which characterized in that, in step (B2), the molar ratio of the second reductant and the transition metal ions is 1:0.05 to 1:40, and/or the molar ratio of the second reductant and the antibody is 2.5:1 to 20:1, and/or the incubation time is 1 h to 24 h.

39. The method of claim 27, which characterized in that, in step (B2), the molar ratio of the second reductant and the transition metal ions is 1:0.4 to 1:100, and/or the molar ratio of the second reductant and the antibody is 0.8:1 to 2.5:1, and/or the incubation time is 0.5 h to 24 h.

40. The method of claim 22, which characterized in that, the transition metal ions are selected from the group consisting of Zn²⁺, Cd²⁺, Hg²⁺, Ni²⁺, Co²⁺ or the combination thereof, optionally, the transition metal ions are Zn²⁺.

41. The method of claim 27 which characterized in that, the second reductant in step (A3) and (B2) is the same as the first reductant in step (A1).

42. The method of claim 27 which characterized in that, the second reductant in step (A3) and the second reductant in step (B2) independently are tris (2-carboxyethyl) phosphine (TCEP).

43. The method of claim 24, which characterized in that, the molar ratio of the oxidant and the antibody in step (A2) is 2:1 to 25:1, optionally, the molar ratio of the oxidant and the antibody in step (A2) is 4:1 to 22:1 or 3:1 to 15:1.

44. The method of claim 24, which characterized in that, the oxidant is Dehydroacetic acid (DHAA).

45. The method of claim 24, which characterized in that, in step (A2), the oxidation temperature is 0° C. to 37° C., and/or the oxidation time is 1 h to 48 h, optionally, the oxidation temperature is 0° C. to 30° C., and/or the oxidation time is 1 h to 5 h.

46. The method of claim 27, which characterized in that, the buffer system of step (A1), (A3) and (B2) independently is selected from a group consisting of MES buffer, Bis-Tris buffer, PIPES buffer, MOPS buffer, BES buffer, HEPES buffer, DIPSO buffer, MOBS buffer, MOPSO buffer, TES buffer, ACES buffer, TAPSO buffer, PBS, Acetate buffer, ADA buffer, BTP buffer, HEPPSO buffer, POPSO buffer, EPPS buffer or Tris buffer,
optionally, the buffer system of step (A1), (A3) and (B2) independently are Bis-Tris buffer, MOPS buffer, BES buffer, HEPES buffer, DIPSO buffer, MOBS buffer, MOPSO buffer, TES buffer, ACES buffer or TAPSO buffer; and/or the concertation of the buffer system is 10 mM to 100 mM, 20 mM to 80 mM, 20 mM to 40 mM, 20 mM to 60 mM, 40 mM to 80 mM or 40 mM to 60 mM; and/or the pH value of the buffer system is 5.5 to 8, preferably, the pH value of the buffer system is 6.7 to 7.4.

47-48. (canceled)

49. The method of claim 27, which characterized in that, the metal chelators in step (B1) and (B3) is Ethylenediaminetetraacetic acid disodium salt (EDTA-2Na).

50. The method of claim 27, which characterized in that, when the first payload units are the first thio-bridging reagent bearing reactive groups, the step (B1) further comprising step of
incubating the metal chelators and the first linker-payloads in the buffer system to react with the reactive groups of the first thio-bridging reagent bearing reactive groups; and/or when the second payload units are the second thio-bridging reagent bearing reactive groups, the step (B3) further comprising step of
incubating the second linker-payloads in the buffer system to react with the reactive groups of the second thio-bridging reagent bearing reactive groups, optionally, introducing the metal chelators.

51. (canceled)

52. The method of any one of claim 27, which characterized in that, the antibody is a monoclonal antibody, a polyclonal antibody, a mono-specific antibody or a multi-specific antibody, optionally, the antibody is IgG1 or IgG4; and/or a linker of the first linker-payload and the second linker payload is selected from any one of which the one terminal can be connected to the reduced thiol group of the antibody or the reactive groups of the thio-bridging reagent, and the other terminal can be connected to the payload; and/or the payload is selected from any one of which contains at least one substituted group allowing a connection from the payload to the linker.

53-54. (canceled)

55. A modified antibody prepared by the method of claim 22.

56. The modified antibody of claim 55, which characterized in that, the modified antibody is the antibody with site-specific modification, optionally, the modified antibody comprises the ADC with D2, the ADC with D4, the ADC with D1, the ADC with D6, the ADC with D3, the ADC with D1+D6, the ADC with D1+D2, the ADC with D1+D4, the ADC with D2+D4, the ADC with D6+D2, the ADC with D6+D1, the ADC with D3+D1, the ADC with D3+D2, the ADC with D0+D6, or the ADC with D0+D2.

57. A pharmaceutical composition comprising an antibody with site-specific modification prepared by the method of claim 22, and at least one pharmaceutically acceptable ingredient.

58. (canceled)

59. A method of preventing or treating a disease in a subject in need thereof, comprising administering to the subject a therapeutically effective amount of the antibody with site-specific modification prepared by the method of claim 22.
